(19) United States
(12) Patent Application Publication
(10) Pub. No.: US 2026/0315544 A1
(43) Pub. Date: Oct. 15, 2026

(54) TRADITIONAL CHINESE MEDICINE COMPOSITION FOR TREATING CHRONIC HEADACHES AND ITS PREPARATION METHOD

(71) Applicant: Hangzhou Hongyu Pharmaceutical Technology Co., Ltd., Zhejiang (CN)

(72) Inventors: Haitong Wan, Zhejiang (CN); Huifen Zhou, Zhejiang (CN); Jiehong Yang, Zhejiang (CN); Haofang Wan, Zhejiang (CN); Xinyu Wu, Zhejiang (CN); Chenxing Zhang, Zhejiang (CN); Zhongcheng Tang, Zhejiang (CN); Wenting Chen, Zhejiang (CN)

(73) Assignee: Hangzhou Hongyu Pharmaceutical Technology Co., Ltd., Zhejiang (CN)

(21) Appl. No.: 19/224,860

(22) Filed: Jun. 1, 2025

(30) Foreign Application Priority Data
Apr. 9, 2025 (CN) 202510435898.3

(51) Int. Cl. A61K 35/64; A61K 36/232; A61K 36/236; A61K 36/488; A61K 36/505; A61K 36/65; A61K 36/8905; A61P 25/06

(52) U.S. Cl. CPC A61K 35/64; A61K 36/232; A61K 36/236; A61K 36/488; A61K 36/505; A61K 36/65; A61K 36/8905; A61P 25/06; A61K 2236/33; A61K 2236/331; A61K 2236/51; A61K 2236/53

(57) ABSTRACT

The present invention relates to a traditional Chinese medicine composition for the treatment of chronic headaches and its preparation method. The composition consists of the following components by weight: 1200-1300 parts of Chuanxiong, 570-670 parts of Bai Shao, 450-550 parts of Ge Gen, 60-500 parts of Vinegar Yanhusuo, 330-420 parts of Honey-fried Jiangan, 320-420 parts of Vinegar Xiangfu, and 200-300 parts of Bai Zhi. The formulation of this traditional Chinese medicine composition is reasonable and simplified, avoiding the waste of medicinal materials. It demonstrates significant efficacy, showing a notable regulatory effect on headache-related indicators and can alleviate central sensitization in chronic migraine rats. Compared to Fluorgelazine and other traditional Chinese medicines for headache relief, it exhibits superior effects and advantages.

### CROSS-REFERENCE TO RELATED APPLICATION

This application claims the priority benefit of China application serial no. 202510435898.3, filed on Apr. 9, 2025. The entirety of the above-mentioned patent application is hereby incorporated by reference herein and made a part of this specification.

### BACKGROUND

### Technical Field

The present invention belongs to the field of natural pharmaceutical chemistry, and specifically relates to a traditional Chinese medicine composition for treating chronic headaches and its preparation method.

### Description of Related Art

Chronic headache is a common neurological disorder, with migraine being the most prevalent type of chronic headache, followed by tension-type headache. Clinical observations have found that chronic headaches are often comorbid with conditions such as anxiety, depression, and sleep disorders. Some studies also suggest that chronic headaches may increase the risk of cognitive dysfunction and cardiovascular diseases. Moreover, the treatment and care costs for chronic headaches are high, which not only severely affects the quality of life of patients but also has a significant impact on the economy and society.

With the development of Western medicine, various theories regarding the pathogenesis of chronic headaches have emerged. These include vascular theories, neuronal theories (such as cortical spreading depression theory and the trigeminovascular theory), and inflammatory mediator theories. Currently, commonly used clinical drugs include nonsteroidal anti-inflammatory drugs (NSAIDs), calcium channel blockers, antiepileptic drugs, β-blockers, calcium channel modulators, and antidepressants, all of which have certain therapeutic and preventive effects for chronic headaches. Among these, NSAIDs are the most widely used for the treatment of migraines, including ibuprofen, diclofenac, aspirin, and naproxen. Their effectiveness has been proven, especially for mild to moderate pain episodes, where 76% of patients experience complete relief during the acute phase of medication. Flunarizine is a preventive drug with strong evidence supporting its use.

Although numerous Western drugs are available for treating chronic headaches, none of them have proven to be a specific cure. Furthermore, due to the long course of chronic headaches, most Western drugs cause adverse reactions such as gastrointestinal issues, extrapyramidal symptoms, cognitive impairment, and long-term use carries risks of liver and kidney damage, thus increasing patient suffering. Therefore, finding a treatment that is long-term, stable, with minimal side effects, and provides good efficacy has gradually become the focus of research in chronic headache treatment. Traditional Chinese medicine (TCM) treats chronic headaches by tailoring the treatment to the specific symptoms and using appropriate prescriptions based on the individual's condition. This approach has shown good clinical efficacy, minimal side effects, and is generally well accepted by patients, with high compliance. Therefore, TCM offers significant advantages in the treatment of chronic headaches. As a result, there is an urgent need to develop new TCM drugs for the treatment of chronic headaches.

### SUMMARY

To address the issues present in the prior art, the objective of the present invention is to provide a traditional Chinese medicine composition for treating chronic headaches and its preparation method. Specifically, this is achieved through the following technical solution:

A traditional Chinese medicine composition for treating chronic headaches, which comprises the following components in parts by weight: Chuanxiong Rhizome, 1200-1300 parts. White Peony Root, 570-670 parts. Kudzu Root, 450-550 parts. Corydalis tuber processed with vinegar, 60-500 parts. Silkworm processed with honey and wheat bran, 330-420 parts. Cyperus Rhizome processed with vinegar, 320-420 parts. and Angelica dahurica Root, 200-300 parts.

A preparation method for a traditional Chinese medicine composition for treating chronic headaches, comprising the following steps:
1) Soak the prescribed amounts of Chuanxiong Rhizoma and Cyperus Rhizome processed with vinegar in 5 times the amount of water as the solvent, and perform steam distillation to extract the volatile oil. The volatile oil is then encapsulated with a saturated β-cyclodextrin solution for later use.
2) Centrifuge the extracted solution from Step 1 to remove impurities and set it aside.
3) Take the residue from Step 1 after volatile oil extraction, add the prescribed amounts of Baishao, Gegen, and honey-fried Bombyx Batryticatus, and use 10 times the amount of water as the solvent. Perform reflux extraction three times, each for 1 hour. Combine the extracted liquids, centrifuge to remove impurities, and concentrate to form the first extract.
4) Pulverize Corydalis tuber processed with vinegar and sieve it through a No. 3 sieve. Take the prescribed amount of Pulverize Corydalis tuber processed with vinegar powder and Angelica dahurica Root, and use 7 times the amount of 60% ethanol as the solvent. Perform reflux extraction twice, each for 1.5 hours. Combine the extracted liquids, filter out impurities using a plate-frame filter, and concentrate to form the second extract.
5) Combine the first and second extracts, spray dry to obtain a dry powder, and add the β-cyclodextrin-encapsulated volatile oil from Step 1. Mix with excipients and use dry granulation to prepare granules.

Further, in Step 1), the soaking time is 0.5 hours, and the steam distillation time is 9.5 hours.

Further, in Step 1), the specific steps for encapsulating the volatile oil with a saturated β-cyclodextrin solution are as follows: mix volatile oil and ethanol in a volume ratio of 1:4 to prepare an ethanol solution of the volatile oil. take β-CD at a material-to-liquid ratio of 1 g:10 mL, add water, and stir at 60° C. for 10 minutes. After cooling to room temperature, prepare the saturated β-CD solution, then transfer it to a constant-temperature magnetic stirrer. Gradually add the volatile oil ethanol solution dropwise at a β-CD to volatile oil volume ratio of 8:1, stirring at 300 r/min and 40° C. for 180 minutes until a suspension is formed. After cooling to room temperature, refrigerate at 4° C. for 24 hours, then filter, wash the resulting inclusion complex once with purified water, and air-dry to obtain the dried encapsulated product.

Further, in Step 3), the relative density of the first extract is 1.10±0.02 g/cm³, and the concentration temperature is 40-50° C.

Further, in Step 4), the reflux extraction temperature is 85° C., the relative density of the second extract is 1.00±0.02 g/cm³, and the concentration temperature is 40-50° C.

Further, in Step 5), the spray drying conditions are as follows: the inlet air temperature is 178° C., the peristaltic pump speed is 12 rpm, and the relative density of the medicinal liquid is 1.07 g/cm³.

Further, in Step 5), the excipients used are dextrin and magnesium stearate, with the amount of dextrin being 5% of the total weight and the amount of magnesium stearate being 0.5% of the total weight.

The traditional Chinese medicine composition of the present invention has a rational formulation with a simplified selection of medicinal ingredients, avoiding the waste of herbal materials. It demonstrates significant efficacy, with a remarkable regulatory effect on headache-related indicators and the ability to alleviate central sensitization in chronic migraine rats.

Compared to flunarizine hydrochloride and other Chinese medicine-based headache relief drugs, it offers even better results and advantages.

### BRIEF DESCRIPTION OF THE DRAWINGS

FIG. 1: HPLC chromatogram of ferulic acid reference solution.

FIG. 2: HPLC chromatogram of ferulic acid in the test solution.

FIG. 3: HPLC chromatogram of paeoniflorin reference solution.

FIG. 4: HPLC chromatogram of paeoniflorin in the test solution.

FIG. 5: HPLC chromatogram of puerarin reference solution.

FIG. 6: HPLC chromatogram of puerarin in the test solution.

FIG. 7: HPLC chromatogram of tetrahydropalmatine reference solution.

FIG. 8: HPLC chromatogram of tetrahydropalmatine in the test solution.

FIG. 9: HPLC chromatogram of europin reference solution.

FIG. 10: HPLC chromatogram of europin in the test solution.

### DESCRIPTION OF THE EMBODIMENTS

The following provides further explanation of the present invention through specific examples to better understand the technical solution.

### Example 1

### Preparation Method of a Traditional Chinese Medicine Composition for Treating Chronic Headaches:

Take Chuanxiong Rhizome, 1240.7 g, and Cyperus Rhizome processed with vinegar, 372.2 g, and use 5 times the amount of water as the solvent. Soak for 0.5 hours, and perform steam distillation for 9.5 hours to extract and collect the volatile oil. Then, encapsulate the volatile oil with a saturated β-cyclodextrin solution and set it aside for later use. The extracted solution is centrifuged to remove impurities, and the resulting liquid is set aside for further use.

The residues of Chuanxiong Rhizome and Cyperus Rhizome processed with vinegar after volatile oil extraction were mixed with White Peony Root 620.3 g, Kudzu Root 496.3 g, and Silkworm processed with honey and wheat bran 372.2 g. Water in tenfold volume was used as the solvent for reflux extraction, conducted three times for 1 hour each. The combined extracts were centrifuged to remove impurities and then concentrated into a clear paste with a relative density of 1.10±0.02 (at 40-50° C.).

Separately, 496.3 g of Corydalis tuber processed with vinegar decoction pieces were pulverized and passed through a No. 3 sieve. The obtained powder, together with Angelica Dahurica Root 620.3 g, was extracted with sevenfold volume of 60% ethanol by reflux at 85° C. for 1.5 hours each time, twice in total. The extracts were purified using a plate-and-frame filter to remove impurities and then concentrated into a clear paste with a relative density of 1.00±0.02 (at 40-50° C.).

Combine the above clear pastes, spray dry to obtain a dry powder, then add the volatile oil encapsulated in β-cyclodextrin solution, and mix with appropriate amounts of dextrin and magnesium stearate. Finally, use dry granulation to prepare the granules.

### Validation Example 1

### 1.1 Quality Inspection

In accordance with the requirements outlined in the 2020 edition of the Pharmacopoeia of the People's Republic of China, the product obtained in Example 1 was subjected to quality tests, including particle size, moisture content, solubility, and dosage uniformity. The results of all tests were in compliance with the specified standards. The detailed results can be found in Table 1-4.

TABLE 1
Particle Size Inspection Results

| Batch Number | Conclusion |
|---|---|
| 20001 | 10.21% |
| 20002 | 9.42% |
| 20003 | 9.85% |
| standard | The total percentage of particles and |
| regulations | powder that cannot pass |
| | through the No. 1 sieve but can pass |
| | through the No. 5 sieve |
| | must not exceed 15.0%. |

TABLE 2
Moisture Content Inspection Results

| Batch Number | Inspection Results |
|---|---|
| 20001 | 3.21% |
| 20002 | 3.62% |
| 20003 | 3.40% |
| standard regulations | Must not exceed 8.0% |

TABLE 3
Solubility Inspection Results

| Batch Number | Conclusion |
|---|---|
| 20001 | qualified |
| 20002 | qualified |
| 20003 | qualified |
| standard | The soluble particles should either completely |
| regulations | dissolve or exhibit slight turbidity. |

TABLE 4
Dosage Uniformity Inspection Results

| Batch Number | Conclusion |
|---|---|
| 20001 | qualified |
| 20002 | qualified |
| 20003 | qualified |
| standard | The number of granule packets (bottles) exceeding |
| regulations | the dosage uniformity limit should not exceed 2, |
| | and no more than 1 packet (bottle) |
| | should exceed the dosage uniformity |
| | limit by more than 1 time. |

### 1.2 Heavy Metal and Arsenic Salt Inspection

The inspection of heavy metals and arsenic salts was conducted using Inductively Coupled Plasma Atomic Emission Spectroscopy (ICP-AES). The results showed that the heavy metal content in all three batches of granules was less than 7 ppm, and the arsenic salt content was less than 1.8 ppm. Therefore, these results were not included in the quality standards.

Preliminary testing confirmed that all three batches met the required specifications.

### 1.3 Microbial Limit

The microbial limits were tested according to the “Microbial Limit Test for Non-Sterile Products” in the 2020 edition of the Chinese Pharmacopoeia, using the dilution method with culture media. After method validation, a pre-test was conducted, and the results showed that the microbial limits meet the specified requirements.

1.4 Content Determination: The content determination was established using High-Performance Liquid Chromatography (HPLC) with ferulic acid, paeoniflorin, puerarin, tetrahydropalmatine, and oxypaeoniflorin as the reference indicators. After method validation, the following HPLC method was developed:

### 1.4.1 Ferulic Acid

Determination was performed according to the High-Performance Liquid Chromatography (HPLC) method referenced in the 2020 Edition of the Chinese Pharmacopoeia, Part 1.

The chromatographic conditions and system suitability test are as follows: Octadecylsilane (C18)-bonded silica gel is used as the packing material, with methanol and 1% acetic acid solution (30:70) as the mobile phase. The detection wavelength is set at 321 nm, and the theoretical plate count, calculated based on the ferulic acid peak, should not be less than 4000.

Preparation of the Standard Solution: Accurately weigh an appropriate amount of ferulic acid reference standard, transfer it to a brown volumetric flask, and add 70% methanol to prepare a solution containing 20 μg of ferulic acid per 1 mL.

Preparation of the Test Solution: Take the contents of the product, grind it finely, weigh about 0.5 g accurately, and place it in a stopper conical flask. Accurately add 50 mL of 70% methanol, stopper the flask, weigh it, heat under reflux for 30 minutes, cool, and reweigh. Make up to the lost weight with 70% methanol, shake well, allow it to settle, and then take the supernatant. Filter the solution to obtain the filtrate.

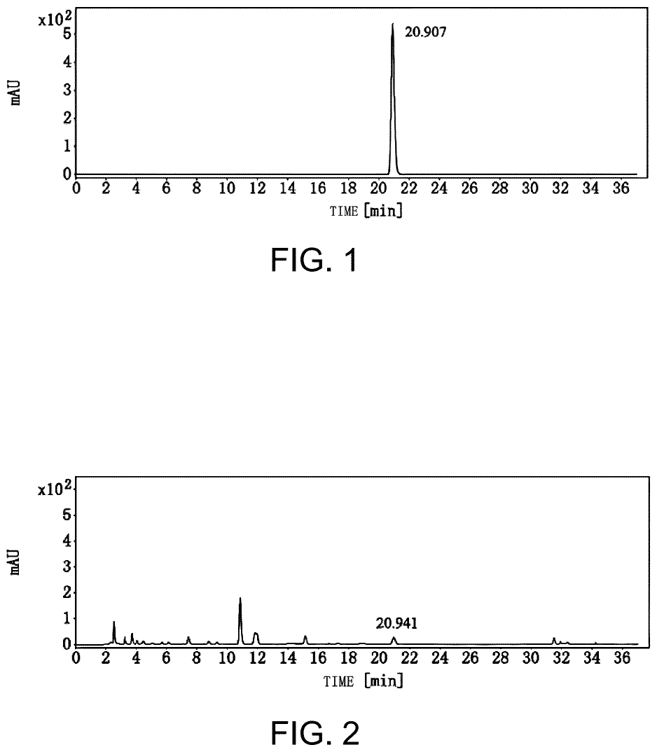

Determination Method: Accurately pipette 10 μL of both the standard solution and the test solution, inject into the HPLC, and measure the ferulic acid content. The chromatograms are shown in FIGS. 1-2.

### 1.4.2 Paeoniflorin

The determination was performed according to the high-performance liquid chromatography (HPLC) method specified in the 2020 edition of the Chinese Pharmacopoeia, Part I.

Chromatographic Conditions and System Suitability Test: A column packed with octadecylsilane-bonded silica gel was used as the stationary phase. the mobile phase consisted of acetonitrile-0.1% phosphoric acid solution (14:86). the detection wavelength was set at 230 nm. The theoretical plate number, calculated based on the paeoniflorin peak, should not be less than 2000.

Preparation of Reference Solution: An appropriate amount of paeoniflorin reference substance was accurately weighed and dissolved in methanol to prepare a solution containing 60 μg per mL.

Preparation of Test Solution: The contents of the product were finely ground, and approximately 0.5 g was accurately weighed and placed into a 50 mL volumetric flask. Then, 35 mL of diluted ethanol was added, and the mixture was subjected to ultrasonic treatment (power: 240 W, frequency: 45 kHz) for 30 minutes. After cooling, the volume was adjusted to the mark with diluted ethanol, mixed well, and filtered. The successive filtrate was collected as the test solution.

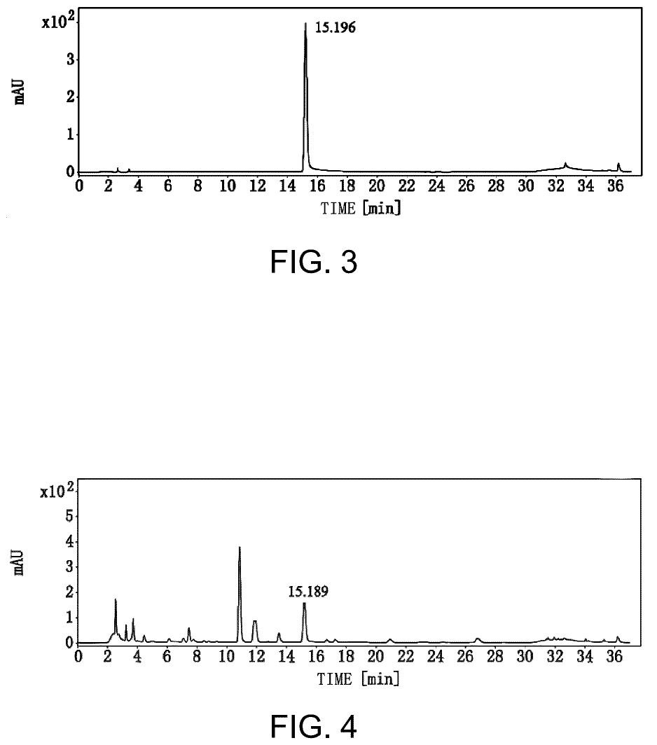

Determination Method: Accurately 10 L of both the reference solution and test solution were injected into the liquid chromatograph for measurement. The HPLC chromatograms are shown in FIGS. 3-4.

### 1.4.3 Puerarin

The determination was performed according to the high-performance liquid chromatography (HPLC) method specified in the 2020 edition of the Chinese Pharmacopoeia, Part I.

Chromatographic Conditions and System Suitability Test: A column packed with octadecylsilane-bonded silica gel was used as the stationary phase. the mobile phase consisted of methanol-water (25:75). the detection wavelength was set at 250 nm. The theoretical plate number, calculated based on the puerarin peak, should not be less than 4000.

Preparation of Reference Solution: An appropriate amount of puerarin reference substance was accurately weighed and dissolved in 30% ethanol to prepare a solution containing 80 g per mL.

Preparation of Test Solution: Approximately 0.1 g of the finely ground powder of the product (sieved through No. 3 sieve) was accurately weighed and placed into a stoppered conical flask. 50 mL of 30% ethanol was precisely added, and the total weight was recorded. The mixture was heated under reflux for 30 minutes, then cooled, and the weight was adjusted with 30% ethanol to compensate for any loss. The solution was mixed well and filtered. The successive filtrate was collected as the test solution.

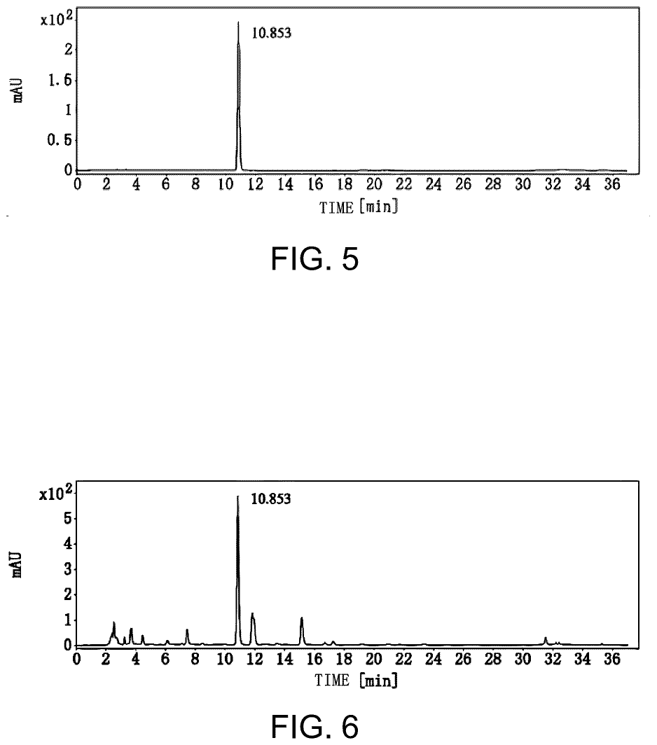

Determination Method: Accurately 10 L of both the reference solution and test solution were injected into the liquid chromatograph for measurement. The HPLC chromatograms are shown in FIGS. 5-6.

### 1.4.4 Tetrahydropalmatine

Chromatographic Conditions and System Suitability Test: A column packed with octadecylsilane-bonded silica gel was used as the stationary phase. the mobile phase consisted of methanol-0.1% phosphoric acid solution (adjusted to pH 6.0 with triethylamine) in a ratio of 55:45. The detection wavelength was set at 280 nm, and the theoretical plate number, calculated based on the tetrahydropalmatine peak, should not be less than 3000.

Preparation of Reference Solution: An appropriate amount of tetrahydropalmatine reference substance was accurately weighed and dissolved in methanol to prepare a solution containing 46 μg per mL.

Preparation of Test Solution: Approximately 0.5 g of the finely ground powder of the product (sieved through No. 3 sieve) was accurately weighed and placed into a flat-bottom flask. Then, 50 mL of a mixed solution of concentrated ammonia test solution and methanol (1:20) was accurately added, and the total weight was recorded. The mixture was cold-soaked for 1 hour and then heated under reflux for another hour. After cooling, the weight was adjusted with the concentrated ammonia-methanol (1:20) solution to compensate for any loss. The solution was mixed well and filtered. 25 mL of the successive filtrate was accurately measured, evaporated to dryness, and the residue was dissolved in methanol. The solution was then transferred to a 5 mL volumetric flask, diluted to volume, mixed well, and filtered. The successive filtrate was used as the test solution.

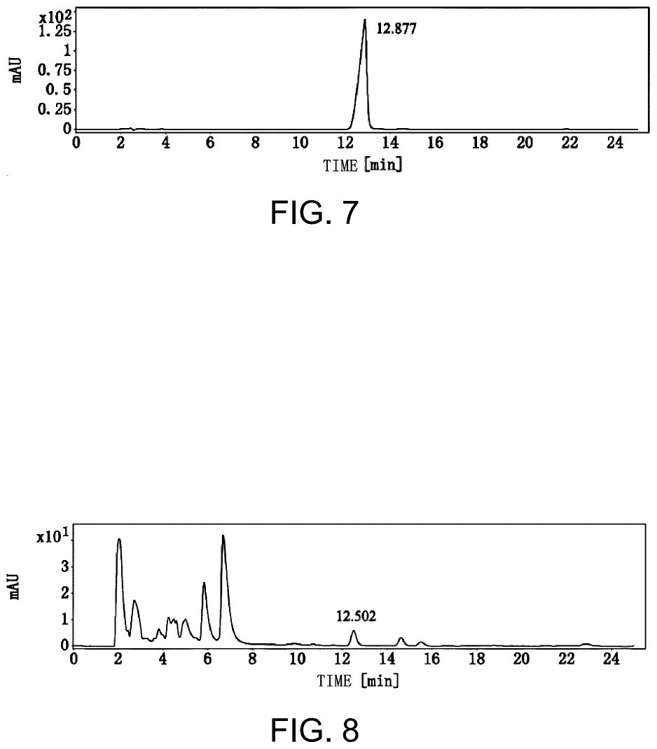

Determination Method: Accurately 10 μL of both the reference solution and test solution were injected into the liquid chromatograph for measurement. The HPLC chromatograms are shown in FIGS. 7-8.

### 1.4.5 Imperatorin

Chromatographic Conditions and System Suitability Test: A column packed with octadecylsilane-bonded silica gel was used as the stationary phase. The mobile phase consisted of methanol-water (55:45). The detection wavelength was set at 300 nm, and the theoretical plate number, calculated based on the imperatorin peak, should not be less than 3000.

Preparation of Reference Solution: An appropriate amount of imperatorin reference substance was accurately weighed and dissolved in methanol to prepare a solution containing 10 μg per mL.

Preparation of Test Solution: Approximately 0.4 g of the finely ground powder of the product (sieved through No. 3 sieve) was accurately weighed and placed into a 50 mL volumetric flask. Then, 45 mL of methanol was added, and the mixture was subjected to ultrasonic treatment (300 W, 50 kHz) for 1 hour. After removal and cooling, methanol was added to bring the volume to the mark. The solution was mixed well, filtered, and the successive filtrate was used as the test solution.

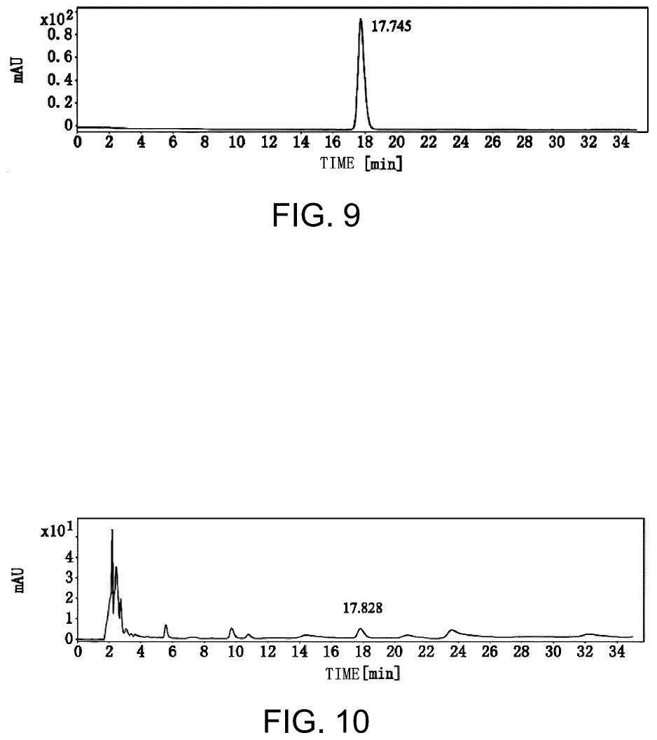

Determination Method: Accurately 10 μL of both the reference solution and test solution were injected into the liquid chromatograph for measurement. The HPLC chromatograms are shown in FIGS. 9-10.

### Validation Example 2: Animal Experiment

Nitroglycerin-Induced Migraine Rat Model: The nitroglycerin-induced migraine model is cost-effective and easy to replicate. It does not require craniotomy, making it convenient for observing the behavior of conscious migraine model rats. This model is currently considered a classical method for studying the effects of drugs on migraines.

### 2.1 Model Preparation

The nitroglycerin-induced migraine rat model was established using the following method: On days 1, 3, 5, 7, and 9 of the experiment, intraperitoneal injections of nitroglycerin solution (10 mg/kg) were administered, for a total of five doses. The nitroglycerin injection solution was prepared freshly before use, with the stock solution (concentration: 5 mg/mL, containing 30% ethanol, 30% propylene glycol, and water) diluted with 0.9% normal saline to a final concentration of 1 mg/mL before injection. The solution was stored protected from light and at a low temperature. Rats in the control group received intraperitoneal injections of an equal volume of normal saline. Successful model establishment was indicated by symptoms such as lethargy, reduced activity, ear flushing, and frequent head-scratching with the forepaws, which are characteristic signs of migraine in rats.

### 2.2 Grouping and Drug Administration

After 7 days of adaptive feeding, SPF-grade rats were randomly divided into a blank control group, a model group, and the following drug administration groups, with a total of 185 groups.

Formulation 1 of the present invention: Chuanxiong Rhizome 30 g, White Peony Root 15 g, Kudzu Root 12 g, Corydalis tuber processed with vinegar 12 g, Silkworm processed with honey and wheat bran 9 g, Cyperus Rhizome processed with vinegar 9 g, and Angelica dahurica Root 6 g.

Formulation 2 of the present invention: Chuanxiong Rhizome 15 g, White Peony Root 7.5 g, Kudzu Root 6 g, Corydalis tuber processed with vinegar 6 g, Silkworm processed with honey and wheat bran 4.5 g, Cyperus Rhizome processed with vinegar 4.5 g, and Angelica Dahurica Root 3 g.

Formulation 3 of the present invention: Chuanxiong Rhizome 60 g, White Peony Root 30 g, Kudzu Root 24 g, Corydalis tuber processed with vinegar 24 g, Silkworm processed with honey and wheat bran 18 g, Cyperus Rhizome processed with vinegar 18 g, and Angelica dahurica Root 12 g.

Control Group 1: a Chinese medicinal formula for the treatment of migraine: Haliotis Shell 20 g, Cassia Seed 20 g, Raw Gypsum 20 g, Mother-of-pearl 20 g, White Peony Root 12 g, Gastrodia Rhizome 9 g, Kudzu Root 15 g, Chinese Gentian 9 g, Chrysanthemum Flower 15 g, Danshen Root 15 g, Red Peony Root 10 g, Moutan Bark 15 g, White Silkworm 9 g, Hemp Seed 9 g, Bush Cherry Seed 9 g, Tortoise Plastron 20 g, Prunella Spike 15 g, Processed Pinellia tuber 9 g, Arisaema tuber processed with bile 9 g, Dried Tangerine Peel 9 g, Grass-leaf Sweetflag Rhizome 10 g, Chinese Scorpion 9 g, Earthworm 9 g, Chuanxiong Rhizome 15 g, Peach Kernel 8 g, Safflower 9 g, Siler Root 9 g, Angelica dahurica Root 10 g, Notopterygium Root 9 g, Vitex Fruit 12 g, Ligustici Rhizome 9 g, Bamboo Shavings 15 g, Reed Rhizome 15 g, Achyranthes Root 15 g, Uncaria Hook 12 g, Processed Aconite Root 10 g, Chinese Angelica Root 15 g, Chinese Wild Ginger 9 g, Atractylodes Rhizome 12 g, Poria 15 g, Antelope Horn 12 g, Corydalis tuber 15 g, Astragalus Root 20 g, Cyperus Rhizome 9 g, Curcuma Root 9 g, Mulberry Fruit 15 g, Mulberry Twig 15 g, Virgate Wormwood Herb 9 g, Radish Seed 9 g, Bupleurum Root 15 g, Loofah 15 g, and HerbaSiegesbeckiae 15 g.

Control Group 2: a Chinese medicine for the treatment of vascular headaches: Kudzu Root 9 g, Earthworm 9 g, Chuanxiong Rhizome 15 g, White Peony Root 12 g, Uncaria Hook 12 g, Chrysanthemum Flower 15 g, Corydalis tuber 15 g, Wild Ginger 9 g, Achyranthes Root 15 g, Angelica dahurica Root 10 g, Saposhnikovia Root 9 g, Scorpion 9 g, Silkworm 9 g, Half-summer Ginger 9 g, Licorice Root 8 g.

Control Group 3: a Chinese medicine composition for the treatment of headache and dizziness and its preparation method: Chuanxiong Rhizome 10-20 parts, Angelica dahurica Root 10-20 parts, Eucommia Bark 10-20 parts, Corydalis tuber 10-20 parts, Silkworm 10-20 parts, Turmeric 10-20 parts, Danshen Root 10-20 parts, PolygoniMultiflori Rhizome 10-20 parts, Lycopus 10-20 parts, Kudzu Root 10-20 parts, Chinese Angelica Root 10-20 parts, Haliotis Shell 10-20 parts, Gastrodia Rhizome 10-20 parts, Astragalus Root 10-20 parts, Cinnamon Twig 10-20 parts, Hangzhou Chrysanthemum 10-20 parts, White Peony Root 10-20 parts.

Control Group 4: a preparation method of Chinese medicine for treating headache: Dihuang 9 g, Fangfeng 9 g, Saposhnikovia Root 9 g, Corydalis tuber 15 g, Gastrodia Rhizome 9 g, Chuanxiong Rhizome 15 g, Silkworm 9 g, Angelica dahurica Root 10 g, Notopterygium Root 9 g, Kudzu Root 15 g, Achyranthes Root 15 g, and Bupleurum Root 15 g.

Control Group 5: a Chinese medicine composition for treating headache: Chuanxiong Rhizome 3 #18 parts, Bupleurum Root 3 #18 parts, Danshen Root 3 #18 parts, Corydalis tuber 5 #25 parts, Angelica dahurica Root 3 #18 parts, White Peony Root 5 #25 parts, White Mustard Seed 3 #18 parts, Cyperus Rhizome 3 #18 parts, Bush Cherry Seed 3 #18 parts, Licorice Root 1 #10 parts.

Control Group 6: a pill formulation for treating headache and its preparation method: Xu Changqing 90 g, Chrysanthemum Flower 80 g, Vitex Fruit 80 g, Gardenia Fruit 80 g, Angelica Dahurica Root 80 g, Ophiopogon Root 150 g, Angelica Pubescens 100 g, Sea Buckthorn 100 g, Notopterygium Root 100 g, Chinese Gentian 50 g, Raw Rehmannia Root 200 g, Fangfeng 80 g, Wine-processed Scutellaria baicalensis 90 g, Wild Ginger 20 g, Basil 90 g, Safflower 80 g, Bupleurum Root 80 g, Gastrodia Rhizome 80 g, Half-summer Ginger 60 g, Astragalus Root 200 g, Kudzu Root 120 g, Chuanxiong Rhizome 80 g, Hawthorn 80 g, Bitter Orange 80 g, Haliotis Shell 120 g, Cang Er Zi 80 g, Saposhnikovia Root 80 g, Wine-processed Rhubarb 80 g, Corydalis tuber 80 g, Papaya 800 g, Silkworm 80 g.

Control Group 7: a pill formulation that can effectively treat headache and its preparation method: Wild Chrysanthemum 90-160 g, Smilax Glabra 150-650 g, Wine-processed Angelica Sinensis 50-120 g, Vitex Fruit 50-120 g, Ophiopogon (without heart) 60-160 g, Antelope Horn Powder 25-60 g, Angelica Pubescens 30-100 g, Gardenia Fruit 100-260 g, Angelica dahurica Root 30-120 g, Notopterygium Root 30-120 g, White Peony Root 60-160 g, Fangfeng 50-120 g, Wine-processed Scutellaria baicalensis 30-100 g, Wild Ginger 15-100 g, Chinese Gentian 30-70 g, Achyranthes Root 50-160 g, Raw Rehmannia Root 90-160 g, Safflower 50-120 g, Bupleurum Root 30-100 g, Astragalus Root 100-360 g, Moutan Bark 60-100 g, Kudzu Root 80-160 g, Gastrodia Rhizome 80-160 g, Half-summer Ginger 30-100 g, Hawthorn 30-120 g, Chuanxiong Rhizome 30-120 g, Bitter Orange 30-120 g, Artemisia Annua 30-120 g, Wine-processed Rhubarb 40-120 g, Haliotis Shell 90-160 g, Ligusticum 50-120 g, Cang Er Zi 30-100 g, Papaya 600-1200 g, Schizonepeta 50-100 g, White Mustard Seed 30-70 g, Cassia Seed 90-160 g, Raw Gypsum 150-650 g, Silkworm 50-100 g, White Peony Root 60-160 g, Licorice Root 30-130 g, Trichosanthes 90-160 g, Snake Skin 20-50 g, Ginseng 15-100 g, Scorpion 20-50 g, Kudzu Root 80-160 g.

Control Group 8: a traditional Chinese medicine for treating neuralgic headache: Chamomile 10-20 g, Cassia Seed 10-15 g, Evodia Fruit 8-12 g, Bamboo Shavings 4-6 g, Chuanxiong Rhizome 10-20 g, Gastrodia Rhizome 8-12 g, Wild Ginger 8-12 g, Corydalis tuber 5-10 g, Vitex Fruit 4-6 g, Cinnamon Twig 5-10 g, Kudingcha (Ilex) 5-15 g, Groundsel 5-10 g, Fritillaria 4-6 g, Mountain Celery Root 1-3 g, Fenugreek 5-7 g, Xu Changqing 4-6 g, Angelica dahurica Root 5-15 g, Tianqi (Panax notoginseng) 8-12 g, Kudzu Root 13-17 g, White Peony Root 8-12 g, Pigweed 5-10 g, and Mountain Plum Blossom Root Bark 4-6 g.

Control Group 9: a traditional Chinese medicine granule for treating menstrual headache: Chuanxiong Rhizome 15 g, Achyranthes Root 15 g, Cyperus Rhizome 12 g, Corydalis tuber 15 g, Motherwort 15 g, Angelica Sinensis 15 g, Brown Sugar 10 g, Rose Flower 15 g, Roasted Barley Malt 15 g, Dendrobium 9 g, Longan Aril 15 g, White Peony Root 12 g, Honey-processed Licorice Root 10 g, Chrysanthemum Flower 15 g, Polygonatum 15 g, Bupleurum Root 15 g, Raw Scutellaria baicalensis 12 g, Roasted Gardenia Fruit 15 g, Pinellia tuber 9 g, Fresh Ginger 8 g, Jujube 10 g, Gastrodia Rhizome 9 g, Uncaria Hook 15 g, Earthworm 9 g, Saposhnikovia Root 9 g, Turmeric 9 g, Tribulus 10 g, Kudzu Root 15 g, and Evodia Fruit 15 g.

Control Group 10: a decoction for treating vascular neurogenic headache and migraine and its preparation method: Bupleurum Root 8 g, Panax Notoginseng 12 g, Chuanxiong Rhizome 9 g, Angelica dahurica Root 8 g, Angelica Pubescens 9 g, Cyperus Rhizome 12 g, Corydalis tuber processed with vinegar 9 g, White Peony Root 15 g, Bush Cherry Seed 12 g, White Mustard Seed 9 g, Licorice Root 8 g, and Albizia Tree Bark 12 g.

Control Group 11: a compound traditional Chinese medicine for treating vascular neurogenic headache: Danshen Root 2-8 parts, Ginseng 2-8 parts, Angelica dahurica Root 8-12 parts, Chuanxiong Rhizome 6-10 parts, Uncaria Hook 8-16 parts, Gastrodia Rhizome 8-12 parts, Ephedra 6-10 parts, Cinnamon 2-6 parts, Earthworm 8-16 parts, Fangfeng 2-8 parts, Cyperus Rhizome 2-6 parts, Kudzu Root 10-20 parts.

Control Group 12: a traditional Chinese medicine for treating refractory headache: Chuanxiong Rhizome 25 g, Angelica dahurica Root 12 g, Processed Cyperus Rhizome 9 g, White Peony Root 25 g, Gastrodia Rhizome 12 g, Silkworm 12 g, Angelica Sinensis 12 g, Wild Ginger 8 g, Fangfeng 9 g, and Earthworm 9 g.

Control Group 13: a traditional Chinese medicine composition for preparing headache treatment drugs: Bupleurum Root 15 g, White Peony Root 12 g, Cyperus Rhizome 9 g, Honey-processed Licorice Root 10 g, Chuanxiong Rhizome 15 g, Kudzu Root 15 g, Angelica dahurica Root 10 g, Angelica Sinensis 15 g, and Papaya 15 g.

Control Group 14: a traditional Chinese medicine composition for treating neuralgic headaches: Chrysanthemum Flower 10-20 parts, Corydalis tuber 10-20 parts, Dai Zhi Shi (Pyrrosia Leaf) 15-25 parts, Tribulus 15-25 parts, Uncaria Hook 10-15 parts, Haliotis Shell 25-35 parts, Atractylodes 15-25 parts, Silkworm 9-15 parts, Cicada Slough 9-15 parts, Vitex Fruit 10-20 parts, Kudzu Root 10-20 parts, Poria 15-25 parts, Danshen Root 9-15 parts, White Peony Root 10-15 parts, Chuanxiong Rhizome 10-15 parts, Schizonepeta 10-15 parts, Scutellaria baicalensis 9-15 parts, Bupleurum Root 10-15 parts, Licorice Root 3-9 parts.

Control Group 15: a traditional Chinese medicine for treating headache caused by kidney deficiency and blood stasis: Rehmannia 25-30 parts, Cornus 15-20 parts, Poria 25-30 parts, Alisma 8-10 parts, Dioscorea 8-10 parts, Acorns 8-10 parts, Peach Kernel 8-10 parts, White Peony Root 15-20 parts, Moutan Bark 8-10 parts, Dragon Bone 12-15 parts, Oyster Shell 25-30 parts, Cinnamon Twig 12-15 parts, Atractylodes 15-20 parts, Licorice Root 4-6 parts, Gastrodia Rhizome 8-10 parts, Pinellia tuber 8-10 parts, Chuanxiong Rhizome 25-30 parts, Angelica Dahurica Root 8-10 parts, Kudzu Root 20-24 parts, Vitex Fruit 12-15 parts, Wild Ginger 4-6 parts, Coptis 4-6 parts, Silkworm 8-10 parts, Safflower 8-10 parts, Centipede 1 piece, Eucommia Bark 8-10 parts, Achyranthes Root 12-15 parts, Mistletoe 12-15 parts, Ground Beetle 8-10 parts, Deer Antler Frost 3-5 parts, Epimedium 25-30 parts, Goji Berries 12-15 parts, and Cinnamon 8-10 parts.

Control Group 16: a traditional Chinese medicine composition for treating cervicogenic headache and its preparation method: Raw Gypsum 25 g, Eriocaulon 9 g, Indigo 20 g, Bupleurum Root 15 g, Pinellia tuber 12 g, Lotus Leaf 15 g, Gastrodia Rhizome 6 g, Dai Zhi Shi (Pyrrosia Leaf) 15 g, Fangfeng 9 g, Centipede 6 g, Red Peony Root 15 g, Astragalus Root 25 g, Earthworm 9 g, Poria 15 g, Motherwort 25 g, Ze Lan 15 g, Safflower 9 g, YiyeXiu 9 g, Saposhnikovia Root 12 g, Xiakucao 9 g, Angelica dahurica Root 12 g, Silkworm 9 g, Angelica Sinensis 15 g, Chuanxiong Rhizome 12 g, White Peony Root 12 g, Rosemary 9 g, Dried Tangerine Peel 10 g, Kudzu Root 18 g, Mulberry Twig 25 g.

Control Group 17: a traditional Chinese medicine composition for treating both acute and chronic migraines and its preparation method: Chuanxiong Rhizome 15 g, Danshen Root 15 g, Saussurea 9 g, Lindera 1 g, Benzoin 1 g, Dried Tangerine Peel 9 g, Curcuma 9 g, Cyperus Rhizome 9 g, Coptis 8 g, Scutellaria baicalensis 12 g, Cinnamon Twig 8 g, Schizonepeta 9 g, Angelica dahurica Root 10 g, Kudzu Root 15 g, Peach Kernel 8 g, Safflower 9 g, Sparganium 9 g, Curcuma zedoaria 9 g, Pangolin Scales 15 g, Raw Astragalus 20 g, White Peony Root 12 g, Red Peony Root 10 g, Gastrodia Rhizome 9 g, Uncaria Hook 12 g, Styrax 15 g, Acorns 10 g, Schizonepeta 9 g, Fangfeng 9 g, Ephedra Root 8 g, Forsythia 9 g, Wild Ginger 9 g, Magnolia Flower 9 g, Peppermint 10 g, Raw Rehmannia 20 g, Tuckahoe 12 g, Rose Flower 15 g, Panax Notoginseng 6 g, Sandalwood 9 g, Tree Sponge 12 g, Motherwort 15 g, Vaccaria Seed 10 g, Ground Beetle 9 g, Sappan Wood 9 g, Dragon's Blood 8 g, and Pinellia tuber 9 g.

Control Group 18: a drug for treating headaches: Bupleurum Root 15 g, White Peony Root 12 g, Fructus Aurantii 9 g, Licorice Root 8 g, Chuanxiong Rhizome 15 g, Dahuang (Rhubarb) 10 g, Cyperus Rhizome 9 g, Raw Gypsum 15 g, Scutellaria baicalensis 12 g, Gentiana 9 g, Uncaria Hook 12 g, Earthworm 9 g, Silkworm 9 g, Angelica dahurica Root 10 g, Vitex Fruit 12 g, Chrysanthemum Flower 15 g, Goji Berries 15 g.

Control Group 19: a traditional Chinese medicine for treating tension-type headaches: Chuanxiong Rhizome 10-20 parts, Bupleurum Root 8-12 parts, Angelica dahurica Root 10-15 parts, Cyperus Rhizome 10-12 parts, Wild Ginger 2-5 parts, Schizonepeta 15-20 parts, White Peony Root 10-20 parts, Xiakucao 10-20 parts, Ziziphus Seed 10-20 parts, Angelica sinensis 10-20 parts, Kudzu Root 25-35 parts, Fructus Aurantii 8-12 parts.

Control Group 20: method for preparing a traditional Chinese medicine for treating headaches: Corydalis 15 g, Gastrodia Rhizome 9 g, Chuanxiong Rhizome 15 g, Silkworm 9 g, Angelica dahurica Root 10 g, Schizonepeta 9 g, Kudzu Root 15 g, Achyranthes 15 g, Bupleurum Root 15 g.

Control Group 21: method for preparing a traditional Chinese medicine for treating headaches: Corydalis 15 g, Gastrodia Rhizome 9 g, Chuanxiong Rhizome 15 g, Silkworm 9 g, Angelica dahurica Root 10 g, Schizonepeta 9 g, Kudzu Root 15 g, Fangfeng 9 g, Achyranthes 15 g, Vitex Fruit 12 g, Platycodon Root 12 g.

Control Group 22: traditional Chinese medicine granules for treating headaches and its preparation method: Chuanxiong Rhizome 600-1000 parts, White Peony Root 100-400 parts, Cyperus Rhizome 100-400 parts, Bupleurum Root 100-400 parts, Scutellaria baicalensis 100-400 parts, Angelica dahurica Root 100-400 parts, Pinellia tuber 100-400 parts, Gastrodia Rhizome 100-400 parts, Corydalis 300-500 parts, Xiakucao 100-400 parts.

Control Group 23: a brain-clearing and pain-relieving preparation: White Peony Root 12 g, Chuanxiong Rhizome 15 g, Kudzu Root 15 g, Corydalis 15 g, Tribulus Terrestris 9 g, Angelica Dahurica Root 10 g, Eleutherococcus 10 g, Xiakucao 15 g.

Control Group 24: a pharmaceutical formulation for alleviating headaches caused by cervical spondylosis and its preparation method: Daizhi Stone 12 g, Fangfeng 10 g, Whole Scorpion 4 g, Red Peony Root 15 g, LiujiNiu 9 g, Kudzu Root 24 g, Astragalus 20 g, Motherwort 30 g, Zeilan 15 g, Safflower 8 g, Goutweed 12 g, Xiakucao 12 g, Angelica dahurica Root 12 g, Poria 30 g, Polygonum 8 g, Angelica sinensis 15 g, South Star 12 g, Chuanxiong Rhizome 12 g, White Peony Root 12 g, Dried Tangerine Peel 9 g, Alisma 12 g, Dragon Bone 30 g, Peach Kernel 12 g, Schizonepeta 9 g, Uncaria Hook 10 g, Roasted Ziziphus Seed 18 g, Pinellia tuber 12 g, Earthworm 9 g, Oyster Shell 30 g, Licorice Root 8 g, Silkworm 6 g.

Control Group 25: a traditional Chinese medicine composition for post-neurosurgical headache care: White Peony Root 12 g, Whole Scorpion 9 g, Turmeric 9 g, Angelica dahurica Root 10 g, Chuanxiong Rhizome 15 g, Red Peony Root 10 g, Salvia 15 g, Xiakucao 15 g, Uncaria Hook 12 g, Gastrodia Rhizome 9 g, Licorice Root 8 g, Prepared Aconite 10 g, Bone-dispersing Herb 9 g, Angelica sinensis 15 g, South Star 9 g, Trillium 9 g, Fangfeng 9 g, Schizonepeta 9 g, Duhuo 9 g, Poria 15 g, Cinnamon 8 g, Corydalis 15 g, Motherwort 15 g, and Turtle Shell 20 g.

Control Group 26: a traditional Chinese medicine pill for treating tension-type headaches and its preparation method: Chuanxiong Rhizome 35-45 parts, Angelica dahurica Root 30-35 parts, White Peony Root 5-10 parts, Gastrodia Rhizome 5-10 parts, Stone Ground Oyster Shell 5-10 parts, Evodia Fruit 5-10 parts, Schizonepeta 3-5 parts, Corydalis 3-5 parts, Ligusticum 3-5 parts, Ligustrum Fruit 3-5 parts.

Control Group 27: an acupoint patch for treating headaches: Chuanxiong Rhizome 10-20 parts, Schizonepeta 10-20 parts, Angelica dahurica Root 10-20 parts, White Peony Root 10-20 parts, White Mustard Seed 10-20 parts, Yue Li Ren 10-15 parts, Evodia Fruit 10-15 parts, Mint 10-15 parts, Cyperus Rhizome 8-12 parts, Bupleurum Root 8-12 parts.

Control Group 28: a medication for treating vascular headaches: Chuanxiong Rhizome 10-15 parts, Gastrodia 12-15 parts, Vitex negundo Fruit 10-13 parts, White Peony Root 10-20 parts, Corydalis Rhizome 10-13 parts, Angelica dahurica Root 15-18 parts, Salvia Root 12-15 parts, Ligusticum 13-16 parts, Peach Kernel 10-15 parts, Safflower 8-12 parts, Bupleurum Root 10-15 parts, Whole Insects 3-5 parts, Gardenia 12-18 parts, Centipede 2-4 parts, Curcuma 10-13 parts, and Honey.

Control Group 29: a medication for treating headaches caused by cervical spondylosis: Pueraria Root 5 g, Bupleurum Root 12 g, White Peony Root 30 g, Chuanxiong Rhizome 30 g, Angelica dahurica Root 12 g, Radix Aconiti 10 g, Apricot Kernel 10 g, Asarum 6 g, Goat Horn (scraped) 20 g, Chuanxiong Rhizome 6 g, Prepared Aconite Root 3 g, Angelica dahurica Root 6 g, Radish 20 g.

Control Group 30: a capsule formulation for treating headaches: Suhexiang 6 g, Angelica Dahurica Root 60 g, Chuanxiong Rhizome 80 g, Angelica Pubescens 80 g, Benzoin 12 g, Rhubarb 120 g, Acorns 50 g, Litsea Cubeba 120 g, Saposhnikovia 80 g, Angelica Sinensis 110 g, White Peony Root 150 g, Bitter Orange 90 g, Red Peony Root 80 g, Saposhnikovia 90 g, Cyperus 90 g, Aquilaria 120 g, Ligusticum 150 g, Sandalwood 40 g, Jixueteng 200 g, Salvia miltiorrhiza 120 g, Peach Kernel 120 g, Pangolin Scales 60 g, Safflower 80 g, Motherwort 200 g, White Mustard Seed 60 g.

Control Group 31: a decoction for effectively treating headaches: Lingyang Jiao Powder 3 g, Gastrodia elata 9 g, Smilax Glabra 30 g, Red Peony Root 8 g, Uncaria 12 g, White Peony Root 12 g, Achyranthes Bidentata 15 g, Scorpion 6 g, Moutan cortex 10 g, Ephedra 8 g, Chuanxiong Rhizome 12 g, Fructus Viticis 9 g, Trichosanthes Fruit 15 g, Cassia Seed 12 g, Asarum 3 g, Angelica Sinensis 15 g, Saposhnikovia 9 g, Atractylodes 12 g, Prepared Rhubarb 20 g, Aconite 9 g, Curcuma 9 g, White Peony Root 9 g, Schizonepeta 12 g, Sweet Potato Leaves 40 g, Saposhnikovia 20 g, Pueraria 12 g, Polygonatum 12 g, Anemarrhena 12 g, Safflower 8 g.

Control Group 32: a novel traditional Chinese medicine composition for treating headaches: Uncaria 200 g, Chuanxiong Rhizome 90 g, Pueraria 120 g, Ephedra 75 g, Asarum 55 g, Prepared Aconite 90 g, Bupleurum 90 g, White Peony Root 110 g, Saposhnikovia 180 g, Angelica Sinensis 120 g, Evodia 35 g, Chicken Blood Vine 130 g, Peach Kernel 120 g, Safflower 80 g, White Peony Root 75 g, Ligusticum 65 g.

Control Group 33: a capsule medication for treating headaches and its preparation method: Uncaria 270 g, Chrysanthemum 80 g, Chuanxiong 95 g, Ephedra 80 g, Ophiopogon (dehusked) 115 g, Asarum 70 g, Aconite 115 g, Gastrodia 12 g, White Peony Root 200 g, Saposhnikovia 200 g, ChuanNiu Xi 95 g, Angelica Sinensis 95 g, Siler 95 g, Salvia miltiorrhiza 115 g, Rehmannia 290 g, Evodia 115 g, Chicken Blood Vine 115 g, Peach Kernel 115 g, Pueraria 120 g, White Peony Root 95 g, Safflower 95 g.

Control Group 34: a health food for patients with stubborn headaches and its preparation method: Notopterygium Root 9 g, White Peony Root 12 g, Vitex Fruit 12 g, Chuanxiong Rhizome 15 g, Angelica dahurica Root 10 g, Platycodon Root 9 g, Cicada Slough 9 g, Scutellaria Root 12 g, Kudzu Root 15 g, Hawthorn Fruit 15 g, Salvia Root 15 g, Frankincense 9 g, Tribulus Terrestris 9 g, Saposhnikovia Root 9 g, Peach Kernel 8 g, Licorice Root 8 g.

Control Group 35: a Chinese herbal formula for relieving headaches: Bupleurum Root 25-35 g, Angelica dahurica Root 25-35 g, Cyperus Rhizome 25-35 g, White Peony Root 6-10 g, Chinese Cherry Pit 6-10 g, White Mustard Seed 2-8 g, Licorice Root 1-3 g, Astragalus Root 2-10 g, Chuanxiong Rhizome 10-12 g, Codonopsis Root 6-10 g, Peach Kernel 2-15 g, Safflower 2-8 g, Earthworm 1-7 g.

Control Group 36: a Chinese herbal composition for treating menstrual headaches: Chuanxiong Rhizome 10-15 g, Cyperus Rhizome 3-8 g, Rose Flower 12-18 g, Eclipta Herb 1-3 g, Ligustrum Fruit 8-12 g, Mulberry Fruit 13-18 g, Corydalis Rhizome 5-10 g, Turmeric 1-5 g, Lycopus 1-3 g, Five-leafed Ginseng Bark 5-10 g, Dogwood Fruit 3-8 g, Gourd 8-13 g, Anemarrhena Rhizome 4-8 g, Dried Ginger 8-12 g, Mugwort Leaf 4-8 g, Polygala Root 1-3 g, White Peony Root 3-8 g, Acorns 1-5 g, Solomon's Seal Root 2-6 g, Morinda Root 5-10 g.

Control Group 37: a medicinal wine for treating headaches and its preparation method: Asarum 3-8 g, Adenophora 15-30 g, Vitex Fruit 10-15 g, Chuanxiong Rhizome 20-30 g, Summer Grass 10-15 g, Phellodendron Bark 5-8 g, Dried Ginger 3-10 g, Sichuan Pepper 2-7 g, Raw Gypsum 13-18 g, Fried Scorpion 5-10 g, White Silkworm 5-12 g, Salvia Root 3-10 g, White Peony Root 5-12 g, Prince Ginseng 3-8 g, Astragalus Root 2-8 g, Kudzu Root 5-10 g, Gastrodia Root 8-15 g, Glutinous Rice 50-100 g, Brewing Yeast appropriate amount.

Control Group 38: a traditional Chinese medicine compound for treating headaches: Corydalis Rhizome 15 g, Angelica dahurica Root 10 g, Morinda Root 15 g, Chuanxiong Rhizome 15 g, Vitex Fruit 12 g, Saussurea Root 9 g, Achyranthes Root 15 g, Kudzu Root 15 g, Scutellaria Root 12 g, Asarum 9 g, Licorice Root 8 g.

Control Group 39: a medicine for treating headaches and its preparation method: Forsythia Fruit 9 g, Honeysuckle Flower 15 g, Angelica dahurica Root 10 g, Hawthorn Fruit 15 g, Isatis Leaf 12 g, Gardenia Fruit 12 g, Rehmannia Root 20 g, Solomon's Seal Root 15 g, Schisandra Fruit 9 g, Lotus Leaf 9 g, Dong Quai 15 g, Chuanxiong Rhizome 15 g, Coptis Root 8 g, Chrysanthemum Flower 15 g, Gourd 15 g, Cicada Slough 15 g, Motherwort 15 g, Summer Grass 15 g, Goji Berry 12 g, Evodia Fruit 9 g, Vitex Fruit 12 g, Corydalis Rhizome 15 g, Mulberry Leaf 20 g, Jujube 10 g, Cocklebur Fruit 9 g, Silkworm 9 g, Bai Mao Gen 15 g, Schizonepeta Herb 9 g, Mint 8 g.

Control Group 40: a traditional Chinese medicine composition for treating neurogenic headaches: Gastrodia Root 4-6 g, Dong Quai 2-4 g, Moutan cortex 8-10 g, Perilla Leaf 4-6 g, Gourd Skin 4-6 g, White Chrysanthemum Flower 2-4 g, Uncaria Hook 10-12 g, Spatholobus Vine 22-26 g, Pink Stone 25-27 g, Fried Gardenia 8-10 g, Scutellaria Root 9-11 g, Leech 8-10 g, Eucommia Bark 6-8 g, Mulberry Leaf 22-24 g, Poria 11-13 g, Rhodiola Root 9-11 g, Fried Scorpion 9-11 g, Bupleurum Root 6-8 g, White Silkworm 6-8 g, Chuanxiong Rhizome 8-10 g, Angelica dahurica Root 8-10 g, Centipede 1-2 g, Kudzu Root 2-4 g, Sour Jujube Seed 2-4 g, Fragrant Jasmine Leaf 15-18 g, Mint 12-14 g, Goat Horn Powder 12-14 g.

Control Group 41: a medicinal preparation for treating headaches: Red Skyflower 15 g, maoyesantiaojin 18 g, Pinellia tuber 5 g, White Peony Root 10 g, Atractylodes Rhizome 30 g, Astragalus Root 10 g, Prince Ginseng 10 g, Dong Quai 10 g, Licorice Root 5 g, Poria 10 g, Poria cocos 10 g, Polygala Root 10 g, Jujube Seed 15 g, Longan Fruit 15 g, Aucklandia Root 5 g, Albizia Flower 20 g, Malt 15 g, Lotus Leaf 10 g, Dogwood Fruit 6 g, Kudzu Root 10 g, Goji Berry 10 g, Angelica dahurica Root 5 g, Saposhnikovia Root 10 g, Leech 5 g, Chuanxiong Rhizome 5 g, Corydalis Rhizome 10 g.

Control Group 42: a medicinal preparation for treating stubborn headaches and its preparation method: Notopterygium Root 9 g, Scutellaria Root 12 g, White Peony Root 12 g, Chuanxiong Rhizome 15 g, Platycodon Root 15 g, Cicada Slough 9 g, Kudzu Root 15 g, Arisaema 9 g, Gastrodia Root 9 g, Frankincense 9 g, Tribulus Terrestris 12 g, Vitex Fruit 12 g, Coptis Root 8 g, Centipede 9 g, Asarum 9 g, Fried Scorpion 9 g, Silkworm 9 g, Licorice Root 8 g.

Control Group 43: a Traditional Chinese Medicine for treating neurogenic headaches: Chuanxiong Rhizome 0-25 g, Angelica dahurica Root 0-35 g, Mugwort Leaf 0-40 g, Gastrodia Root 0-20 g, Centipede 0-15 g, Large-leaf Uncaria Hook 0-35 g, Kudzu Root 0-25 g, Corydalis Rhizome 0-20 g, Vitex Fruit 0-20 g.

Control Group 44: A Chuanxiong Qingnao Analgesic Capsule and Its Preparation Method. The formulation includes Chuanxiong Rhizome 25%, Gastrodia Root 10%-12%, Asarum 8%-10%, Angelica dahurica Root 10%-12%, Tianqi Ginseng 10%-12%, Kudzu Root 13%-15%, White Peony Root 18%-20%.

Control Group 45: A Traditional Chinese Medicine Composition for Treating Neurogenic Headache. The formulation includes Fried Chuanwu 10 g, Chuanxiong Rhizome 25 g, Angelica dahurica Root 30 g, Asarum 6 g, Fried Scorpion 6 g, Centipede 2 pieces, Gastrodia Root 15 g, Silkworm 10 g, White Peony Root 30 g, Fried Licorice Root 10 g, White Tribulus 30 g, Magnetite 30 g, Mother-of-Pearl 30 g, Curcuma Root 15 g, Salvia Root 30 g, Jujube Seed 15 g, Achyranthes Root 10 g, Spatholobus Vine 30 g, Mustard Seed 10 g.

Control Group 46: A Traditional Chinese Medicine Preparation for Treating Vascular Neurogenic Headache. The formulation includes Chuanxiong Rhizome 10-15 g, Saussurea Root 10-15 g, Angelica dahurica Root 10-15 g, Vitex Fruit 10-15 g, Saposhnikovia Root 8-12 g, Notopterygium Root 8-12 g, Kudzu Root 12-18 g, Bupleurum Root 12-18 g, White Peony Root 12-18 g, Scutellaria Root 10-15 g, Pinellia tuber 7-12 g, Salvia Root 10-15 g, Safflower 5-8 g, Dong Quai 10-15 g, Licorice Root 5-7 g.

Control Group 47: A Traditional Chinese Medicine for Treating Refractory Headache. The formulation includes Chuanxiong Rhizome 20-30 g, Angelica dahurica Root 10-15 g, Processed Cyperus Rhizome 6-10 g, White Peony Root 20-30 g, Gastrodia Root 10-15 g, Silkworm 10-15 g, Dong Quai 10-15 g, Asarum 5-10 g, Saposhnikovia Root 6-10 g, Earthworm 6-10 g.

Control Group 48: A Traditional Chinese Medicine Composition for Treating Neurogenic Headache. The formulation includes Gastrodia tuber 4-6 g, Angelica Sinensis Root 2-4 g, Moutan Bark 8-10 g, Perilla Leaf 4-6 g, Snakegourd Peel 4-6 g, White Chrysanthemum Flower 2-4 g, Uncaria Hook 10-12 g, Caulis PolygoniMultiflori 22-26 g, Haliotidis Concha 25-27 g, Stir-fried Gardenia Fruit 8-10 g, Scutellaria Root 9-11 g, Hirudo 8-10 g, Eucommia Bark 6-8 g, Mulberry Leaf 22-24 g, Poria Cocos with Hostwood 11-13 g, Rhodiola Rosea Root 9-11 g, Scorpion 9-11 g, Bupleurum Root 6-8 g, Bombyx Batryticatus 6-8 g, Chuanxiong Rhizome 8-10 g, Angelica dahurica Root 8-10 g, Centipede 1-2 pieces, Kudzu Root 2-4 g, Semen ZiziphiSpinosae 2-4 g, Clerodendranthus fragrans (Roxb.) Vent. Leaf 15-18 g, Mint Leaf 12-14 g, Goat Horn Powder 12-14 g.

Control Group 49: A Traditional Chinese Medicine Composition and Its Preparation Method for Treating Headache. The formulation includes Chuanxiong Rhizome 15 g, Gastrodia tuber 9 g, Angelica dahurica Root 10 g, White Peony Root 12 g, Corydalis tuber 15 g, Scorpion 9 g

Control Group 50: A Traditional Chinese Medicine for Treating Headache. The formulation includes Bupleurum Root 28-33 g. Scutellaria Root 18-22 g. Kudzu Root 28-33 g. Chuanxiong Rhizome 18-22 g. Angelica dahurica Root 18-22 g. Chrysanthemum Flower 28-32 g. White Peony Root 28-32 g. Poria Cocos 18-22 g. Mint Leaf 12-17 g. Saposhnikovia Root 12-17 g. Licorice Root 3-7 g.

Control Group 51: A Traditional Chinese Medicine Composition and Its Preparation Method for Treating Headache. The formulation includes TRhizomaSmilacisGlabrae 20 g, Antelope Horn Powder 12 g, Gastrodia tuber 9 g, Red and White Peony Roots 10 g, Achyranthes Root 15 g, Moutan Bark 15 g, Scorpion 9 g, Chuanxiong Rhizome 15 g, Leonurus Seed 9 g, Cassia Seed 15 g, Snakegourd Fruit 15 g, Angelica Sinensis Root 15 g, Saposhnikovia Root 9 g, Prepared Rhubarb 15 g, Atractylodes macrocephala Rhizome 12 g, Curcuma Root tuber 9 g, Angelica Dahurica Root 10 g, Sweet Potato Leaf 10 g, Schizonepeta Tenuifolia 9 g, Kudzu Root 15 g, Polygonatum Rhizome 15 g, Anemarrhena Rhizome 12 g

Control Group 52: A Proprietary Chinese Medicine for Preventing and Treating Vascular Neurogenic Headache. The formulation includes Atractylodes macrocephala Rhizome 10-20 g, Angelica dahurica Root 3-6 g, Gastrodia tuber 10-15 g, Ephedra Herb 5-15 g, Chuanxiong Rhizome 10-20 g, Uncaria Hook 10-20 g, Corydalis tuber 20-30 g, Vitex Fruit 20-30 g, Kudzu Root 20-30 g

Control Group 53: A Traditional Chinese Medicine Composition and Its Preparation Method for Treating Menstrual Headache. The formulation includes ARhizomaAcoriTatarinowii 10 g, Benzoin 9 g, HerbaLycopi 9 g, Chuanxiong Rhizome 15 g, Ramulus cinnamomi 8 g, Salvia Miltiorrhiza Root 15 g, Styrax 9 g, Immature Bitter Orange 9 g, Saposhnikovia Root 9 g, Peach Kernel 9 g, Safflower 9 g, Tangerine Peel 9 g, PericarpiumCitriReticulataeViride 9 g, Szechwan Chinaberry Fruit 15 g, Angelica dahurica Root 10 g, Notopterygium Root 9 g, Gastrodia tuber 9 g, Radix notoginseng 6 g, Mint Leaf 9 g, Dried Ginger 8 g, Fructus PiperisLongi 9 g, Aquilariae Lignum resinatum 9 g, Cyperus Rhizome 9 g, Curcuma Root tuber 12 g, Corydalis tuber 15 g.

Control Group 54: A Medicine for Treating Headache and Its Preparation Method. The formulation includes Chrysanthemum Flower 15 g, Bupleurum Root 15 g, Chuanxiong Rhizome 15 g, Astragalus Root 20 g, Angelica Sinensis Root 15 g, Tribulus Fruit 10 g, Lycium barbarum Fruit 9 g, Bombyx Batryticatus 9 g, White Peony Root 12 g, Licorice Root 8 g, Scorpion 9 g, Kudzu Root 15 g, Centipede 9 g.

Control Group 55: A Proprietary Chinese Medicine for Preventing and Treating Vascular Neurogenic Headache. The formulation includes Atractylodes macrocephala Rhizome 10-20 g, Angelica dahurica Root 3-6 g, Gastrodia tuber 10-15 g, Ephedra Herb 5-15 g, Chuanxiong Rhizome 10-20 g, Uncaria Hook 10-20 g, Corydalis tuber 20-30 g, Vitex Fruit 20-30 g, Kudzu Root 20-30 g.

Control Group 56: A Medicine for Treating Neurogenic Headache. The formulation includes Ligusticum sinense Oliv Rhizome 10-15 parts, Vitex Fruit 10-15 parts, Angelica dahurica Root 8-12 parts, Chuanxiong Rhizome 10-15 parts, Red Peony Root 10-15 parts, White Peony Root 10-20 parts, Angelica Sinensis Root 10-30 parts, Salvia Miltiorrhiza Root 10-30 parts, Poria Cocos with Hostwood 10-30 parts, Uncaria Hook 10-20 parts, Scrophularia Root 10-20 parts, Gastrodia tuber 8-15 parts, Earthworm 5-10 parts, Corydalis tuber 10-15 parts, Concha Margaritifera usta 10-30 parts, Licorice Root 3-5 parts.

Control Group 57: A Traditional Chinese Medicine Capsule for Treating Headache. The formulation includes White Peony Root 12 g, Cyperus Rhizome 9 g, Scutellaria Root 12 g, Licorice Root 8 g, Mint Leaf 9 g, Bupleurum Root 15 g, Chrysanthemum Flower 15 g, Chuanxiong Rhizome 15 g, Poria Cocos 15 g, Angelica Sinensis Root 15 g, Angelica dahurica Root 10 g, Moutan Bark 15 g.

Control Group 58: A Medicine for Treating Vascular Headache and Its Preparation Method. The formulation includes Chuanxiong Rhizome 12-15 parts, Gastrodia tuber 12-15 parts, Vitex Fruit 10-13 parts, White Peony Root 15-18 parts, Corydalis tuber 10-13 parts, Angelica dahurica Root 15-18 parts, Salvia Miltiorrhiza Root 12-15 parts, Ligusticum sinense Oliv. Rhizome 13-16 parts, Bupleurum Root 9-12 parts, Scorpion 2-5 parts, Gardenia Fruit 13-16 parts, Zedoary Rhizome 10-13 parts, Licorice Root 3-5 parts.

Control Group 59: A Traditional Chinese Medicine Composition and Its Preparation Method for Treating Menstrual Headache. The formulation includes Acorus tatarinowii Rhizome 10 g, Benzoin 9 g, Dipterocarpus turbinatus Gaertn. f. 9 g, Chuanxiong Rhizome 15 g, Cinnamon Twig 8 g, Salvia Miltiorrhiza Root 15 g, Styrax 9 g, Aurantium immaturus 9 g, Saposhnikovia divaricata Root 9 g, Peach Kernel 9 g, Safflower 9 g, Tangerine Peel 9 g, PericarpiumCitriReticulataeViride 9 g, Toosendan Fruit 9 g, Angelica dahurica Root 10 g, Notopterygium Root 9 g, Gastrodia tuber 9 g, Notoginseng Root 9 g, Mint Leaf 9 g, Dried Ginger 8 g, Long Pepper 9 g, Agarwood 9 g, Cyperus Rhizome 9 g, Curcuma AromaticaSalisb. 9 g, Corydalis tuber 15 g.

Control Group 60: A Traditional Chinese Medicine Composition and Its Preparation Method for Treating Headache. The formulation includes Angelica dahurica Root 10 g, Chuanxiong Rhizome 15 g, Styrax 15 g, Benzoin 9 g, Acorns Tatarinowii Rhizome 10 g, Dipterocarpus turbinatus Gaertn. f. 9 g, Rheum palmatum Root 8 g, Angelica Sinensis Root 15 g, White Peony Root 12 g, Red Peony Root 10 g, Aurantium immaturus 9 g, Cyperus Rhizome 9 g, Notopterygium Root 9 g, Agarwood 9 g, Dalbergia Odorifera T. Chen 9 g, Salvia Miltiorrhiza Root 15 g, Peach Kernel 9 g, Safflower 9 g, Leonurus Heterophyllus Sweet 15 g, Pangolin Scale 12 g.

Control Group 61: A FangfengDuzhong Compound Decoction for Treating Headache. The formulation includes Saposhnikovia divaricata Root 9 g, Eucommia Bark 9 g, Chuanxiong Rhizome 15 g, Spatholobus suberectus Dunn 15 g, Angelica Sinensis Root 15 g, Taxillus chinensis (DC.) Danser 9 g, Cassia Seed 15 g, Schizonepeta tenuifolia Briq. 9 g, Gastrodia tuber 9 g, Angelica dahurica Root 10 g, Asarum Herb 9 g, Scorpion 9 g, Mint Leaf 10 g, Corydalis tuber 15 g, Cyperus Rhizome 9 g.

Control Group 62: Soft Capsule for Treating Vascular Headache. The formulation includes Chuanxiong Rhizome 15 g, Bupleurum Root 15 g, Angelica dahurica Root 10 g, White Mustard Seed 9 g, White Peony Root 12 g, Cyperus Rhizome 9 g, Prunus japonica Thunb. 9 g, Licorice Root 8 g.

Control Group 63: A Traditional Chinese Medicine Water Pill Formula for Treating Headache. The formulation includes Chuanxiong Rhizome 15 g, Mint Leaf 9 g, Kaempferia galanga L. 9 g, Angelica dahurica Root 10 g, Asarum Herb 9 g, Schizonepeta tenuifolia Briq. 9 g, Saposhnikovia divaricata Root 9 g, Notopterygium Root 9 g, Ligusticum sinense Rhizome 9 g, Vitex Fruit 12 g, Xanthium sibiricum 9 g, Pueraria lobata 15 g, Gardenia Fruit 12 g, Fermented Soybean 15 g, Forsythia suspensa 9 g, Chrysanthemum Flower 12 g, Scutellaria baicalensis 12 g, Platycodon grandiflorus 15 g, Oroxylum indicum 9 g, Cyperus Rhizome 9 g, Medicated Leaven 9 g, Raphanus Sativus 9 g, Ginger 8 g, Licorice Root 8 g.

Control Group 64: A Traditional Chinese Medicine for Treating Migraine. The formulation includes Chuanxiong Rhizome 15 g, Asarum Herb 9 g, Salvia miltiorrhiza Root 15 g, Angelica dahurica Root 10 g, Pueraria Root 15 g, Scorpion 9 g, Polygonum multiflorum Root 15 g, Cyperus Rhizome 9 g, Gardenia Fruit 12 g, Atractylodes Rhizome 12 g, Angelica Sinensis Root 15 g, RhizomaSmilacisGlabrae 20 g, Centipede 9 g, White Peony Root 12 g, Radix rehmanniae 20 g, Coptis Root 8 g, Saposhnikovia Root 9 g.

Control Group 65: A Traditional Chinese Medicine Formula for Treating Headache. The formulation includes Chuanxiong Rhizome 30 g, White Peony Root 15 g, Semen ZiziphiSpinosae 15 g, Pueraria Root 15 g, Gastrodia tuber 10 g, Bombyx Batryticatus 10 g, White Mustard Seed 3 g, Asarum Herb 3 g.

Control Group 66: A Traditional Chinese Medicine for Treating Vascular Neuralgia. The formulation includes Schizonepeta tenuifolia 9 g, Saposhnikovia divaricata Root 9 g, Angelica dahurica Root 10 g, Asarum Herb 9 g, Chrysanthemum Flower 15 g, Vitex Fruit 12 g, Angelica Sinensis Root 15 g, Chuanxiong Rhizome 15 g, White Peony Root 12 g, Bupleurum Root 15 g, Scutellaria baicalensis Root 12 g, Achyranthes bidentata Root 15 g, Earthworm 9 g, Scorpion 9 g, Bombyx Batryticatus 9 g.

Control Group 67: A Traditional Chinese Medicine Composition for Treating Neuralgic Headache. The formulation includes Chrysanthemum Flower 10-20 parts, Corydalis tuber 10-20 parts, Haematitum 15-25 parts, Tribulus Terrestris Fruit 15-25 parts, Uncaria rhynchophylla Hook 10-15 parts, Concha Margaritifera usta 25-35 parts, Atractylodes macrocephala Rhizome 15-25 parts, Bombyx Batryticatus 9-15 parts, Cicada Slough 9-15 parts, Vitex Fruit 10-20 parts, Pueraria Lobata Root 10-20 parts, Poria Cocos 15-25 parts, Salvia Miltiorrhiza Root 9-15 parts, White Peony Root 10-15 parts, Chuanxiong Rhizome 10-15 parts, Schizonepeta Tenuifolia 10-15 parts, Scutellaria baicalensis Root 9-15 parts, Bupleurum Root 10-15 parts, Liquorice Root 3-9 parts.

Control Group 68: A Traditional Chinese Medicine Composition for Treating Severe Headache and Its Preparation Method. The formulation includes Bupleurum Root 12 g, Cyperus Rhizome 10 g, Cinnamon Twig 10 g, Tangerine Peel 10 g, Moutan Bark 10 g, Peony Root 10 g, Licorice Root 10 g, Atractylodes macrocephala Rhizome 10 g, Rehmannia Root 10 g, Poria Cocos 10 g, Apricot Kernel 10 g, Polygonum Multiflorum Root 10 g, Pinellia tuber 10 g, Chuanxiong Rhizome 30 g, Angelica Sinensis Root 30 g, Asarum Herb 15 g, Angelica dahurica Root 15 g, Notopterygium Root 20 g, Vitex Fruit 10 g.

Control Group 69: A Traditional Chinese Medicine for Treating Headache Due to Kidney Deficiency and Blood Stasis Syndrome. The formulation includes Prepared Rehmannia Root 25-30 parts, Cornus officinalis Fruit 15-20 parts, Poria Cocos 25-30 parts, Alisma Orientalis Rhizome 8-10 parts, Dioscorea opposita Rhizome 8-10 parts, Acorus calamus Rhizome 8-10 parts, Peach Kernel 8-10 parts, White Peony Root 15-20 parts, Moutan Bark 8-10 parts, Dragon's Bone 12-15 parts, Oyster Shell 25-30 parts, Cinnamon Twig 12-15 parts, Atractylodes macrocephala Rhizome 15-20 parts, Licorice Root 4-6 parts, Gastrodia tuber 8-10 parts, Pinellia tuber 8-10 parts, Chuanxiong Rhizome 25-30 parts, Angelica dahurica Root 8-10 parts, Pueraria Lobata Root 20-24 parts, Vitex Fruit 12-15 parts, Asarum Herb 4-6 parts, Coptis chinensis Root 4-6 parts, Bombyx Batryticatus 8-10 parts, Safflower 8-10 parts, Scolopendra 1 piece, Eucommia ulmoides Bark 8-10 parts, Achyranthes Bidentata Root 12-15 parts, Taxillus Chinensis Twig 12-15 parts, Eupolyphaga SeuSteleophaga 8-10 parts, Deer Antler Frost 3-5 parts, Epimedium Herb 25-30 parts, Lycium barbarum Fruit 12-15 parts, Cinnamon Bark 8-10 parts.

Control Group 70: A Traditional Chinese Medicine Formulation for Treating Neuropathic Headache. The formulation includes: Antelope Horn 3-5 parts, Gastrodia tuber 4-6 parts, Chuanxiong Rhizome 4-6 parts, Bupleurum Root 4-6 parts, Cyperus Rhizome 3-5 parts, Fructus Aurantii 3-5 parts, Spine Date Seed 4-6 parts, Red Peony Root 3-5 parts, White Peony Root 3-5 parts, Angelica sinensis Root 3-5 parts, Raw Dragon's Bone 6-8 parts, Haliotis Shell 6-8 parts, Acorus calamus Rhizome 3-5 parts, Curcuma aromatica Rhizome 3-5 parts, Asarum Herb 0.5-1.5 parts, Poria cocos 3-5 parts, Atractylodes macrocephala Rhizome 3-5 parts, Vitex Fruit 3-5 parts, Angelica dahurica Root 3-5 parts, Ligusticum sinense Root 3-5 parts, Scorpion 1-3 parts, Scolopendra 0.5-1.5 parts, Earthworm 3-5 parts, Amomum villosum Fruit 3-5 parts, Amomum Kravanh Fruit 3-5 parts, Medicated Leaven 3-5 parts, Prepared Rehmannia Root 4-6 parts, Turtle Shell Glue 6-8 parts, Donkey—hide Glue 6-8 parts, Yellow Rice Wine 14-18 parts, Rock Sugar 14-18 parts.

Control Group 71: A Traditional Chinese Medicine Formulation for Treating Neuropathic Headache. The formulation includes: White Peony Root 10-50 parts, Cicada Slough 6-30 parts, Angelica dahurica Root 6-30 parts, Pueraria Lobata Root 10-40 parts, Cinnamon Twig 6-20 parts, Asarum Herb 2-6 parts, Chuanxiong Rhizome 5-20 parts, Vitex Fruit 5-30 parts, Raw Licorice Root 4-20 parts.

Control Group 72: A Traditional Chinese Medicine Granule for Treating Migraine, named XifengZhitong Granules, and its Preparation Method. The formulation includes: Angelica sinensis Root 15 g, Chuanxiong Rhizome 15 g, White Peony Root 12 g, Gastrodia tuber 9 g, Angelica dahurica Root 10 g, Cynanchum paniculatum Root 9 g, Corydalis tuber 15 g, Cyperus Rhizome 9 g.

Control Group 73: A Traditional Chinese Medicine Composition for Treating Neuropathic Headache. The formulation includes: Angelica Sinensis Root 10-20 g, Salvia Miltiorrhiza Root 15-40 g, Peach Kernel 5-18 g, Safflower 3-15 g, White Peony Root 20-50 g, Licorice Root 3-10 g, Trogopterus Dung 5-15 g, Myrrh 5-18 g, Corydalis tuber 15-40 g, Chuanxiong Rhizome 5-25 g, Angelica dahurica Root 3-18 g, Scutellaria baicalensis Root 5-20 g, Chrysanthemum Flower 10-30 g, Notopterygium Root 10-25 g.

Control Group 74: A Traditional Chinese Medicine Composition for Treating both Tension and Migraine Headaches and its Preparation Method. The formulation includes: Chuanxiong Rhizome 15 g, Salvia miltiorrhiza Root 15 g, Aucklandia Root 9 g, Lysimachia foenum—graecum Herb 9 g, Benzoin 9 g, Tangerine Peel 9 g, Curcuma Aromatica Rhizome 9 g, Cyperus Rhizome 12 g, Coptis chinensis Rhizome 8 g, Scutellaria baicalensis Root 12 g, Cinnamon Twig 8 g, Notopterygium Root 9 g, Angelica dahurica Root 10 g, Pueraria lobata Root 15 g, Peach Kernel 9 g, Safflower 9 g, Sparganium stoloniferum Rhizome 9 g, Curcuma zedoaria Rhizome 9 g, Pangolin Scales 9 g, Raw Astragalus Root 20 g, White Peony Root 12 g, Red Peony Root 10 g, Gastrodia tuber 9 g, Uncaria rhynchophylla 12 g, Styrax 9 g, Acorus tatarinowii Rhizome 10 g, Schizonepeta tenuifolia Herb 9 g, Saposhnikovia divaricata Root 9 g, Ephedra Root 8 g, Forsythia suspensa Fruit 9 g, Asarum Herb 9 g, Magnolia biondii Flower Bud 15 g, Mentha haplocalyx Herb 10 g, Raw Rehmannia Root 20 g, Toona Sinensis Fruit 15 g, Rose Flower 15 g, Notoginseng Root 6 g, Dalbergia odorifera Wood 9 g, Garden Burnet Root 15 g, Motherwort Herb 15 g, Vaccaria segetalis Seed 9 g, Eupolyphaga SeuSteleophaga 9 g, Sappan Wood 10 g, Dragon's Blood 9 g, Pinellia ternata Rhizome 9 g.

Control Group 75: A Traditional Chinese Medicine for Treating Neurovascular Headaches. The formulation includes: Pueraria lobata Root 30-40 g, Chuanxiong Rhizome 18-20 g, Angelica dahurica Root 18-20 g, Chrysanthemum Flower 15-18 g, Corydalis tuber 10-15 g, Uncaria rhynchophylla 20-30 g, Achyranthes bidentata Root 15-20 g, Asarum Herb 3-6 g, Scutellaria baicalensis Root 10-15 g, Ligusticum sinense Root 10-15 g, Alisma orientalis Rhizome 10-15 g.

Control Group 76: A Traditional Chinese Medicine Composition for Treating Neurogenic Headaches. The formulation includes: Angelica sinensis Root 10-20 g, Salvia Miltiorrhiza Root 15-40 g, Peach Kernel 5-18 g, Safflower 3-15 g, White Peony Root 20-50 g, Licorice Root 3-10 g, Trogopterus Dung 5-15 g, Myrrh 5-18 g, Corydalis tuber 15-40 g, Chuanxiong Rhizome 5-25 g, Angelica dahurica Root 3-18 g, Scutellaria baicalensis Root 5-20 g, Chrysanthemum Flower 10-30 g, Notopterygium Root 10-25 g.

Control Group 77: Thrombolytic and Lipid-lowering Pills. The formulation includes: Bupleurum Root 15 g, Pueraria lobata Root 15 g, Salvia miltiorrhiza Root 15 g, Astragalus Root 20 g, Cinnamon Twig 8 g, Moutan Bark 15 g, White Peony Root 12 g, Red Peony Root 10 g, Angelica sinensis Root 15 g, Stir-fried Chuanxiong Rhizome 15 g, Earthworm 9 g, Cassia Seed 15 g, Scorpion 9 g, Bombyx Batryticatus 9 g, Centipede 9 g, Benzoin 1 g, Gastrodia tuber 9 g, Radix codonopsis 15 g, Achyranthes bidentata Root 15 g, Pangolin Scales 9 g, Notopterygium Root 9 g, Typhonium giganteum Rhizome 9 g, Angelica pubescens Root 9 g, Agkistrodon acutus 10 g, Zaocys dhumnades 10 g, Asarum Herb 9 g, Poria cocos with Hostwood 18 g, Lonicera japonica Flower 15 g, Gardenia Fruit 9 g, Licorice Root 8 g.

Control Group 78: A traditional Chinese medicine for treating neurovascular headaches: Pueraria lobata Root 30-40 g, Chuanxiong Rhizome 18-20 g, Angelica dahurica Root 18-20 g, Chrysanthemum Flower 15-18 g, Corydalis tuber 10-15 g, Uncaria rhynchophylla 20-30 g, Achyranthes bidentata Root 15-20 g, Asarum Herb 3-6 g, Scutellaria baicalensis Root 10-15 g, Ligusticum sinense Root 10-15 g and Alisma orientalis Rhizome 10-15 g.

Control Group 79: An oral preparation for treating headaches and its preparation method: Chuanxiong Rhizome 200-500, Stir-fried White Peony Root 300-500, Angelica dahurica Root 200-500, Scorpion 30-50, Centipede 30-50, Piper Longum Fruit 20-60, Bombyx Batryticatus 50-160, Stir-fried Licorice Root 60-120, Vitex Fruit 120-180, Ligusticum sinense Root 100-160, Asarum Herb 24-35, Corydalis tuber 100-160, Bupleurum Root 80-120, Mentha Haplocalyx Herb 60-80, Medicinal Excipients 20-53.

Control Group 80: A traditional Chinese medicine preparation for treating headaches and its preparation method: Chuanxiong Rhizome 15 g, Gastrodia tuber 9 g, Corydalis tuber 15 g, Angelica dahurica Root 12 g, Red Peony Root 10 g, Prepared Rehmannia Root 20 g, Angelica sinensis Root 15 g, Safflower 9 g, Salvia miltiorrhiza Root 15 g, Saposhnikovia divaricata Root 9 g, Asarum Herb 9 g, Pueraria lobata Root 15 g, Lonicera japonica Flower 12 g.

Control Group 81: Traditional Chinese medicine for treating stubborn headaches: Scorpion 5 g, Angelica dahurica Root, Notopterygium Root, Chuanxiong Rhizome each 12 g, White Peony Root 24 g, Concha Margaritifera usta 30 g, Corydalis tuber 15 g, Earthworm 12 g, Licorice Root 5 g.

Control Group 82: Traditional Chinese medicine for treating muscle contraction headache: Bupleurum Root and Cyperus Rhizome each 10 g, White Peony Root and Pueraria lobata Root each 30 g, Licorice Root 6 g, Chuanxiong Rhizome 25 g, Glossy Privet Fruit, Caulis PolygoniMultiflori and Salvia miltiorrhiza Root each 15 g, Tribulus Terrestris Fruit, Vitex Fruit and Chrysanthemum Flower each 10 g.

Control Group 83: Traditional Chinese medicine for treating liver Qi stagnation headache: Bupleurum Root 15 g, Codonopsis pilosula Root, Scutellaria baicalensis Root, Pinellia tuber, Chuanxiong Rhizome, Corydalis tuber, Uncaria rhynchophylla, Cyperus rotundus Rhizome each 10 g, White Peony Root 12 g, Licorice Root 6 g.

Control Group 84: A new method for treating headaches with Chaihu, Chuanxiong, Baizhi, Danggui, and Chrysanthemum: Bupleurum Root 15 g, Chuanxiong Rhizome 15 g, Angelica dahurica Root 10 g, Angelica Sinensis Root 15 g, Cyperus Rhizome 9 g, Chrysanthemum Flower 15 g, Salvia Miltiorrhiza Root 15 g, Magnetite 20 g, Red Peony Root 10 g, White Peony Root 12 g, Caulis Spatholobi 15 g, Leonurus Japonicus Herb 15 g, Uncaria rhynchophylla 12 g, Licorice Root 8 g.

Control Group 85: A new method for treating headaches with Tianma, Cicada Molt, Licorice, and ZhiBaihuTongjingXitong: Chuanxiong Rhizome 15 g, Gastrodia tuber 9 g, Cicada Slough 9 g, Licorice Root 8 g, Processed Typhonium giganteum Rhizome 9 g, Uncaria rhynchophylla 12 g, Bombyx Batryticatus 9 g, Earthworm 9 g, White Peony Root 12 g, Pueraria lobata Root 15 g.

Control Group 86: A preparation for treating tension-type headaches and its preparation method: Cyperus Rhizome 2.5-4.5 parts, Red Peony Root 3-4.5 parts, Chuanxiong Rhizome 2.5-4.5 parts, Angelica sinensis Root 3-4.5 parts, Corydalis tuber 2.5-4 parts, Asarum Herb 0.5-1.5 parts, Angelica dahurica Root 1.5-3 parts, Scutellaria baicalensis Root 1.5-3 parts, Bupleurum Root 1-3 parts.

Control Group 87: Oral traditional Chinese medicine preparation for treating vascular headaches and migraines and its preparation method: Chuanxiong Rhizome 15 g, Bupleurum Root 15 g, Angelica dahurica Root 10 g, Cyperus Rhizome 9 g, White Peony Root 12 g, Prunus humilis Bunge Seed 9 g, Sinapis alba Seed 10 g, Licorice Root 8 g.

Control Group 88: Traditional Chinese medicine for treating cervical spondylosis and its preparation method: Gastrodia tuber 9 g, Uncaria rhynchophylla 12 g, Chuanxiong Rhizome 15 g, Corydalis tuber 15 g, Pueraria lobata Root 15 g, Angelica dahurica Root 10 g.

Control Group 89: Traditional Chinese medicine composition for treating persistent headaches: Stir-fried Peach Kernel 10-15 g, Safflower 10-15 g, Angelica Sinensis Root 15-20 g, Chuanxiong Rhizome 10-15 g, Fructus Aurantii 15-20 g, Angelica dahurica Root 15-20 g, Rehmannia glutinosa Root 20-25 g, Bupleurum Root 15-20 g, Red Peony Root 15-20 g, Platycodon grandiflorum Root 15-20 g, Pueraria Lobata Root 15-20 g, Asarum Herb 3-5 g, Vitex trifolia Var. Simplicifolia Cham. 10-15 g, Licorice Root 6-10 g, White Peony Root 15-20 g, Prepared Rehmannia Root 15-20 g, Evodia rutae carpa Fruit 10-15 g, Atractylodes lancea Rhizome 10-15 g, Salvia Miltiorrhiza Root 30-40 g, Astragalus Membranaceus Root 20-25 g, Atractylodes macrocephala Koidz. 15-20 g, Saposhnikovia divaricata Root 10-15 g.

Control Group 90: Traditional Chinese medicine for treating headaches and trigeminal neuralgia: Earthworm 4.76 g, Processed Typhonium giganteum Rhizome 6.76 g, Chuanxiong Rhizome 11.26 g, Arisaema Cum Bile 6.76 g, Red Peony Root 6.76 g, Asarum Herb 2.82 g, Corydalis tuber 6.76 g, Pubescent Angelica Root 6.76 g, Safflower 5.4 g, Vitex trifolia Var. Simplicifolia Cham. 6.76 g, Peach Kernel 5.4 g, Angelica dahurica Root 6.76 g, Angelica sinensis Root 6.76 g, Scorpion 4.76 g, Gastrodia tuber 6.76 g, Bombyx Batryticatus 4.76 g.

Control Group 91: Traditional Chinese medicine formula for treating migraine, including capsules, pills, and powders, and its preparation method: Raw White Peony Root 15-25 parts, Honey-fried Licorice Root 5-15 parts, Chrysanthemum 5-15 parts, Chuanxiong Rhizome 15-25 parts, Mentha haplocalyx 5-15 parts, Angelica dahurica Root 10-20 parts, Notopterygium Root 5-15 parts, Asarum Herb 4-6 parts, Bupleurum Root 10-20 parts, Vinegar-processed Cyperus Rhizome 5-15 parts, Curcuma aromatica Salisb. 5-15 parts, Moutan Cortex 5-15 parts, Scutellaria baicalensis Root 5-15 parts, Ligusticum sinense Oliv. 5-15 parts, Vitex Trifolia Var. Simplicifolia Cham. 5-15 parts, Corydalis tuber 10-20 parts, Cynanchum paniculatum 10-20 parts.

Control Group 92: A Traditional Chinese Medicine preparation for treating hypertension: Forsythia Suspensa Fruit 9 g, Tangerine Peel 9 g, Safflower 9 g, Earthworm 9 g, Eclipta prostrata 15 g, Red Peony Root 10 g, Ophiopogon Japonicus Root 18 g, Scrophularia ningpoensis Root 12 g, Pinellia ternata Rhizome 9 g, Pueraria Lobata Root 15 g, Salvia Miltiorrhiza Root 15 g, Chuanxiong Rhizome 15 g, Motherwort Herb 15 g, Astragalus Membranaceus Root 20 g, Corydalis tuber 15 g, Plantago Seed 10 g, Mulberry Twig 10 g, Lycium chinense Mill. Root-Bark 12 g, Taxillus Chinensis Danser 12 g, Codonopsis pilosula Root 15 g, Angelica dahurica Root 10 g, Phellodendron chinense Schneid. 12 g, Cassia Seed 15 g, Turtle Shell 20 g, Licorice Root 8 g.

Control Group 93: A Traditional Chinese Medicine capsule for clearing the brain and relieving pain and its preparation method: Chuanxiong Rhizome 25%, Gastrodia tuber 10%-12%, Asarum Herb 8%-10%, Angelica dahurica Root 10%-12%, Panax notoginseng 10%-12%, Pueraria lobata Root 13%-15%, White Peony Root 18%-20%.

Control Group 94: A medicine for treating cervical spondylosis and its preparation method: Cinnamon Twig 80-120 parts, White Peony Root 100-180 parts, Pueraria lobata Root 200-340 parts, Chuanxiong Rhizome 100-160 parts, Zedoary Turmeric Root—tuber 80-120 parts, Corydalis tuber 100-160 parts, Stir-fried Jujube Seed 170-230 parts, Astragalus membranaceus Root 170-220 parts, Angelica sinensis Root 90-150 parts, Poria 170-210 parts, Bupleurum Root 90-110 parts, Scutellaria baicalensis Root 80-130 parts.

Control Group 95: A Traditional Chinese Medicine for treating migraines and its preparation method: Glabrous Greenbrier Rhizome 20 g, Glehnia littoralis Root 15 g, Gastrodia tuber 9 g, White Peony Root 12 g, Spatholobus suberectus Dunn 15 g, Achyranthes Bidentata Blume 15 g, Moutan Cortex 15 g, Scorpion 9 g, Ginger 8 g, Chuanxiong Rhizome 15 g, Schisandra Chinensis 9 g, Cassia Seed 15 g, Trichosanthes kirilowii Maxim. 15 g, Angelica Sinensis Root 15 g, Saposhnikovia divaricata Root 9 g, Phellodendron chinense Schneid. 12 g, Atractylodes macrocephalaKoidz. 12 g, Angelica dahurica Root 10 g, Glossy Privet Fruit 9 g, Mulberry Leaf 20 g, Schizonepeta Tenuifolia Briq. 9 g, Pueraria Lobata Root 15 g, Exocarpium citri Grandis 9 g, Anemarrhena asphodeloides Bunge 12 g, Pubescent Angelica Root 9 g.

Control Group 96: A Traditional Chinese Medicine for treating migraines: Lingyangjiao 15 g, Chuanxiong 15 g, Quanxie 9 g, Wugong 9 g, Baishao 12 g, Jiangcan 9 g, Baizhi 10 g, Gouteng 12 g, Sanqi 6 g, Dihuang 20 g, ZhiGancao 10 g.

Control Group 96: A traditional Chinese medicine for treating migraine: Antelope Horn 15 g, Chuanxiong Rhizome 15 g, Scorpion 9 g, Centipede 9 g, White Peony Root 12 g, Bombyx Batryticatus 9 g, Angelica dahurica Root 10 g, Uncaria rhynchophylla 12 g, Panax notoginseng 6 g, Rehmannia Root 20 g, Honey-fried Licorice Root 10 g.

Control Group 97: A traditional Chinese medicine preparation for treating migraine: Chuanxiong Rhizome 15 g, Angelica dahurica Root 10 g, Saposhnikovia divaricata Root 9 g, Notopterygium incisum Ting ex H. T. Chang 9 g, Vitex Trifolia L. var. Simplicifolia Cham. 12 g, Pueraria Lobata Root 15 g, Scorpion 9 g, Bombyx Batryticatus 9 g, Gastrodia tuber 9 g.

Control Group 98: Treatment of 68 cases of chronic tension-type headaches with a modified headache formula: Chuanxiong Rhizome 10 g, Angelica dahurica Root 10 g, Notopterygium incisum Ting ex H. T. Chang 10 g, Ligusticum sinense Oliv. 10 g, Bupleurum Root 10 g, Corydalis tuber 10 g, Cyperus Rotundus Rhizome 10 g, White Peony Root 15 g, Pueraria Lobata Root 15 g, Asarum Herb 5 g, Licorice Root 5 g.

Control Group 99: Traditional Chinese Medicine treatment for chronic headaches: Chuanxiong Rhizome 15 g, Angelica dahurica Root 10 g, Bombyx Batryticatus 9 g, Gastrodia tuber 9 g, Pueraria Lobata Root 15 g, Asarum Herb 9 g, Corydalis tuber 15 g, Bupleurum Root 15 g, Scorpion 9 g.

Control Group 100: Efficacy observation of Chuanxiong Shuqiao Granules in treating 43 cases of tension-type headache. Chuanxiong Shuqiao Granules: Chuanxiong Rhizome 15 g, Corydalis tuber 15 g, Pueraria Lobata Root 15 g, Ligusticum sinense Root 9 g, Achyranthes Bidentata Root 15 g, White Peony Root 12 g, Angelica dahurica Root 10 g, Salvia Miltiorrhiza Root 15 g, Bupleurum Root 15 g, Chinese Gentian Root 9 g, Chrysanthemum Flower 15 g, Gastrodia tuber 9 g, Uncaria rhynchophylla 12 g.

Control Group 101: A randomized parallel-controlled study on HuoxueZhitong Decoction for treating blood stasis headache. HuoxueZhitong Decoction: Peach Kernel 12 g, Angelica Sinensis Root 12 g, Angelica dahurica Root 12 g, Olibanum 12 g, Red Peony Root 12 g, Myrrh 12 g, Prepared Rehmannia Root 15 g, Fructus Aurantii 15 g, Corydalis tuber 15 g, White Peony Root 15 g, Cyperus Rhizome 15 g, Chuanxiong Rhizome 10 g, Licorice Root 6 g, Safflower 6 g.

Control Group 102: Experience of Famous Old Chinese Medicine Doctors in Treating Diseases Series—Lu Yongbing's Treatment of Headaches—GejuXiongma Decoction: Pueraria lobata Root 30 g, Hangzhou Chrysanthemum Flower (Chrysanthemum morifolium) 15 g, White Peony Root 15 g, Bombyx Batryticatus 15 g, Rehmannia glutinosa Root 15 g, Salvia miltiorrhiza Root 15 g, Chuanxiong Rhizome 10 g, Scutellaria baicalensis Root 10 g, Gastrodia tuber 10 g, Angelica dahurica Root 10 g, Licorice Root 5 g.

Control Group 103: Treatment of tension-type headache with the method of soothing the liver, alleviating urgency, dispelling wind, and relieving pain: Chuanxiong Rhizome 15 g, Notopterygium Root 15 g, Angelica dahurica Root 15 g, Schizonepeta tenuifolia 15 g, Pueraria lobata Root 15 g, Bupleurum Root 15 g, Cyperus Rhizome 15 g, Jujube Seed 15 g, Licorice Root 15 g, Angelica sinensis Root 15 g, Asarum Herb 5 g, White Peony Root 20 g.

Control Group 104: Clinical observation of self-formulated headache prescription combined with Sibelium for the treatment of migraine. Self-formulated headache prescription: Chuanxiong Rhizome 15 g, Poria 15 g, Angelica dahurica Root 15 g, Gastrodia tuber 15 g, Angelica sinensis Root 15 g, Salvia miltiorrhiza Root 15 g, Ligusticum sinense Root 10 g, Pueraria lobata Root 10 g, Bupleurum Root 10 g, White Peony Root 10 g, Cyperus Rhizome 10 g, Vitex Fruit 10 g, Evodia rutae carpa Fruit 6 g, Licorice Root 6 g.

Control Group 105: Integrated Chinese and Western medicine treatment for 40 cases of primary headache: Gastrodia tuber 15 g, Chrysanthemum 12 g, Prepared Rehmannia Root 15 g, White Peony Root 15 g, Bupleurum Root 9 g, Scutellaria baicalensis Root 12 g, Cyperus Rhizome 6 g, Angelica dahurica Root 15 g, Gardenia Fruit 8 g, Concha Margaritifera usta 30 g, Alisma orientale Rhizome 15 g, Polygonatum sibiricum Rhizome 30 g, Chuanxiong Rhizome 15 g, Asarum Herb 3 g, Corydalis tuber 12 g

Control Group 106: Efficacy of Chuanxiong and Baizhi Decoction in treating migraine and its effect on EEG and brainstem evoked potentials: Chuanxiong Rhizome 20 g, Angelica dahurica Root 10 g, Red Peony Root 10 g, White Peony Root 10 g, Corydalis tuber 10 g, Selfheal Spike 10 g, Vitex trifolia Var. Simplicifolia Cham. 10 g, Ligusticum sinense Oliv. 10 g, Mentha HaplocalyxBriq. 10 g, Chrysanthemum morifolium Ramat. 10 g, Bupleurum Root 10 g, Asarum Herb 3 g, Saposhnikovia divaricata Root 5 g, Poria cocos with Hostwood 10 g, Pueraria lobata Root 15 g.

Control Group 107: A randomized parallel controlled study on the treatment of migraine with Qingnao Headache Decoction:
Chuanxiong Rhizome 10 g, Angelica dahurica Root 10 g, Red Peony Root 10 g, White Peony Root 10 g, Corydalis tuber 10 g, Selfheal Spike 10 g, Vitex Fruit 10 g, Ligusticum sinense Root 10 g, Mentha haplocalyx 5 g, Chrysanthemum 10 g, Bupleurum Root 10 g, Asarum Herb 3 g, Saposhnikovia divaricata Root 5 g, Poria Cocos with Hostwood 10 g, Pueraria lobata Root 15 g.

Control Group 108: Treatment of 360 cases of vascular headache with TongluoJiejuan Decoction: White Peony Root 30 g, Uncaria Stem with Hooks (added later) 30 g, Chuanxiong Rhizome 20 g, Danshen Root 20 g, Pueraria lobata Root 20 g, Saposhnikovia divaricata Root 15 g, Corydalis tuber 15 g, Angelica dahurica Root 10 g, Liquorice Root 10 g, Earthworm 10 g, Scorpion 10 g, Turmeric Root—tuber 10 g, Fingered Citron 10 g, Asarum Herb 6 g.

Control Group 109: Treatment of 108 cases of vascular headache with ShuganJiejuan Decoction: Bupleurum Root 12 g, Cyperus Rhizome 12 g, Angelica dahurica Root 12 g, Chuanxiong Rhizome 12 g, Corydalis tuber 15 g, White Peony Root 15 g, Licorice Root 3 g.

Control Group 110: Quick-acting Headache Decoction combined with Strychnine and Scorpion Capsule for treating 67 cases of migraine. Quick-acting Headache Decoction: Chuanxiong Rhizome 15-30 g, Pueraria Lobata Root 15-30 g, Achyranthes Root 15-30 g, Astragalus Root 15-30 g, Safflower 5-10 g, Gastrodia tuber 10-20 g, Asarum 6-10 g, Angelica Dahurica 6-10 g, Ledebouriella Root 6-10 g, Corydalis tuber 6-15 g, Turmeric Root-tuber 6-15 g, Angelica Sinensis 6-15 g, White Peony Root 6-15 g, Licorice Root 6 g.

Control Group 111: TongqiaoAnnao Decoction for treating 168 cases of vascular headache. TongqiaoAnnao Decoction: Danshen Root 30 g, Chuanxiong Rhizome 30 g, Red Peony Root 15 g, White Peony Root 15 g, Cyperus rotundus Rhizome 20 g, Bupleurum Root 10 g, Angelica dahurica Root 10 g, Bombyx Batryticatus 10 g, Notopterygium Root 12 g, 5 Chrysanthemum 10 g, Borneol 0.2 g (separately wrapped and dissolved).

Control Group 112: LiqiHuoxue Decoction for treating 34 cases of menstrual headache. LiqiHuoxue Decoction: Bupleurum Root 15 g, Earthworm 15 g, Corydalis tuber 15 g, Cyperus Rhizome 15 g, Chinese Angelica Root 15 g, Angelica dahurica Root 15 g, Peach Kernel 10 g, Scorpion 10 g, Gastrodia tuber 10 g, White Peony Root 20 g, Chuanxiong Rhizome 20 g, Danshen Root 20 g, Dragon's Bone 20 g, Oyster Shell 20 g.

Control Group 113: Clinical observation of “LigiTongqiao Decoction” for treating 33 cases of vascular headache. LiqiTongqiao Decoction: Danshen Root 30 g, Chuanxiong Rhizome 20 g, White Peony Root 20 g, Cyperus Rhizome 15 g, Corydalis tuber 15 g, Poria 25 g, Saposhnikovia divaricata Root 25 g, White Mustard Seed 10 g, Notopterygium Root 15 g, Bupleurum Root 15 g, Angelica dahurica Root 10 g, Sanqi Powder 5 g.

Control Group 114: One case of “Qu Feng Zhi Tong Formula” for treating headache. Qu Feng Zhi Tong Formula: Chuanxiong Rhizome 15 g, Pueraria Lobata Root 20 g, Angelica Dahurica Root 20 g, White Peony Root 20 g, Pinellia ternata (Processed) 10 g, Scutellaria baicalensis Root 5 g, Raw Licorice Root 6 g, Scorpion 5 g, Centipede 1 piece, Bombyx Batryticatus 10 g.

Control Group 115: Clinical efficacy observation of Professor Tao Genyu's self-formulated prescription for treating tension-type headache. Prescription: Bupleurum Root 12 g, Chuanxiong Rhizome 20 g, Chinese Angelica Root 15 g, White Peony Root 15 g, Bighead Atractylodes Rhizome 12 g, Gypsum Fibrosum 20 g, Silktree Albizia Bark 30 g, Asarum Herb 6 g, Pueraria Lobata Root 20 g, Angelica dahurica Root 12 g, Bombyx Batryticatus 6 g, Scorpion 6 g.

Control Group 116: Treatment of cerebrovascular spasm headache in 60 cases with GegenJuhuangXiongma Decoction. Prescription: Pueraria Lobata Root 40 g, Chrysanthemum morifolium 15 g, Chuanxiong Rhizome 6 g, Gastrodia tuber 10 g, White Peony Root 20 g, Licorice Root 5 g, Bombyx Batryticatus 15 g, Angelica dahurica Root 8 g, ScutellariaBaicalensis Root 10 g, Rehmannia glutinosa Root 20 g, Danshen Root 20 g.

Control Group 117: Treatment of migraine (56 cases) with Chaihu Chuanxiong Decoction. Prescription: Bupleurum Root 15 g, Chinese Angelica Root 15 g, Angelica dahurica Root 15 g, Bombyx Batryticatus 15 g, Pueraria Lobata Root 15 g, White Peony Root 15 g, Chuanxiong Rhizome 30 g, Asarum Herb 7.5 g, Medicinal Evodia Fruit 10 g, Licorice Root 10 g.

Control Group 118: Optimization and Evaluation of the Standard Decoction and Granule Formulation of the Classical Chuanxiong Rhizome 37.3 g, White Peony Root 18.65 g, Angelica Dahurica Root 1.865 g, Chinese Dwarf Cherry Seed 3.73 g, Cyperus Rhizome 7.46 g, Bupleurum Root 3.73 g, White Mustard Seed 11.19 g, Licorice Root 3.73 g.

Control Group 119: Clinical Observation on the Treatment of Chronic Headache with the Self-Formulated Yuanhu Dulong Decoction: Corydalis Rhizome 10 g, Pheretima 10 g, Angelica dahurica Root 10 g, Bupleurum Root 10 g, Acorus tatarinowii Rhizome 10 g, Safflower 10 g, Peach Kernel 10 g, Pangolin Scale 10 g, Chinese Angelica Root 15 g, Rehmannia glutinosa Root 15 g, Chuanxiong Rhizome 30 g, White Peony Root 30 g, Centipede 1 piece (ground and taken with decoction), Scorpion 2 g (ground and taken with decoction).

Control Group 120: Efficacy Observation of the TongmaiZhitung Formula in the Treatment of Phlegm-Stasis Type Migraine in 321 Cases: Astragalus Root 20 g, Chinese Angelica Root 12 g, White Peony Root 30 g, Chuanxiong Rhizome 30 g, Hirudo 6 g, Angelica dahurica Root 9 g, Pheretima 6 g, Cinnamon Twig 6 g, Bupleurum Root 6 g, Pueraria Lobata Root 15 g, Gastrodia tuber 9 g, Cynanchumpaniculatum 30 g, Rhubarb 30 g, Chrysanthemum morifolium 9 g, Vitex trifolia var. simplicifolia 9 g.

Control Group 121: Qin Yuehao's Experience in Treating Female Migraine from the Perspective of Wind-Phlegm-Stasis: 10 g of Chuanxiong Rhizome, 20 g of Stir-Fried White Peony Root, 10 g of White Mustard Seed, 10 g of Angelica dahurica Root, 20 g of Glabrous Greenbrier Rhizome, 15 g of Danshen Root, 10 g of Bombyx Batryticatus, 10 g of Xanthium Sibiricum.

Control Group 122: Clinical Observation of 60 Cases of Migraine Treated with a Self-Formulated Headache Prescription: Bupleurum Root 15 g, Chuanxiong Rhizome 10 g, Angelica Dahurica Root 10 g, Saposhnikovia divaricata Root 10 g, Asarum Herb 4 g, Pueraria Lobata Root 30 g, Mulberry Twig 30 g, White Peony Root 25 g, Scorpion 5 g, Centipede 2 pieces, Danshen Root 20 g, Chinese Angelica Root 10 g, Astragalus Root 30 g, Honey—Fried Licorice Root 10 g.

Control Group 123: Treatment of Migraine with Traditional Chinese Medicine Headache Powder: Chuanxiong Rhizome 20 g, Angelica dahurica Root 20 g, Asarum Herb 3 g, Notopterygium Root 20 g, Saposhnikovia divaricata Root 20 g, Uncaria Stem with Hooks 15 g, Honey—Fried Scorpion 15 g, Danshen Root 20 g, Earthworm 20 g, Bombyx Batryticatus 20 g, Medicinal Evodia Fruit 20 g, Gypsum Fibrosum 30 g, Red Peony Root 15 g, Cyperus Rhizome 20 g.

Control Group 124: Experience of Dr. Zhang Zilin's Use of San Pian Tang in the Treatment of Migraine: White Peony Root 15 g, Chuanxiong Rhizome 30 g, Prunus Dulcis 3 g, Bupleurum Root 3 g, White Mustard Seed 9 g, Cyperus Rhizome 6 g, Licorice Root 3 g, Angelica Dahurica Root 1.5 g.

Control Group 125: XiongzhiShaoyao Decoction for the treatment of 38 cases of migraine: Chuanxiong Rhizome 15 g, Angelica dahurica Root 15 g, Raw White Peony Root 15 g, Bombyx Batryticatus 10 g, Licorice Root 7.5 g.

Control Group 126: Syndrome-based treatment of 41 cases of vascular headache: Chuanxiong Rhizome 15 g, Angelica dahurica Root 15 g, Chinese Angelica Root 15 g, Bombyx Batryticatus 10 g, Chrysanthemum 10 g, White Peony Root 15 g, Ligusticum sinense Root 10 g, Vitex Fruit 10 g, Licorice Root 6 g.

Control Group 127: Clinical observation of 84 cases of migraine treated with self-formulated Chuanxiong Tianma Decoction: Chuanxiong Rhizome 30-40 g, Gastrodia tuber 15 g, NotopterygiumRoot 12 g, Angelica dahurica Root 15 g, Asarum Herb 9 g, Earthworm 12 g, Vinegar—processed Corydalis tuber 15 g, White Peony Root 15 g, Licorice Root 10 g.

Control Group 128: Seven Traditional Chinese Medicine Pain Relief Formulas: Pain Relief Formula: Gastrodia tuber 15 g, Chuanxiong Rhizome 15 g, Angelica dahurica Root 15 g, Aconitum carmichaeli Root 15 g, Corydalis tuber 15 g.

Control Group 129: Self-formulated Chuanxiong GuizhiTianmaBaizhi Decoction for the Treatment of Vascular Neurogenic Headache in 53 Cases: Chuanxiong GuizhiTianmaBaizhi DecoctionChuanxiong Rhizome 20 g, Chinese Angelica Root 15 g, White Peony Root 15 g, Salvia Miltiorrhiza Root 15 g, Gastrodia tuber 10 g, Bombyx Batryticatus 10 g, Saposhnikovia Root 10 g, Angelica dahurica Root 10 g, Pinellia tuber 10 g, Bupleurum Root 8 g, Asarum Herb 4 g, Licorice Root 4 g.

Control Group 130: Clinical Case of Stubborn Internal Injury Headache Treatment: Honey-fried Astragalus Root 200 g, Chuanxiong Rhizome 30 g, Rehmannia Root 30 g, Safflower 6 g, Angelica dahurica Root 6 g, Chinese Angelica Root 15 g, Corydalis tuber 12 g, Prepared Cyperus Rhizome 10 g, Asarum Herb 3 g.

Control Group 131: Clinical Study on Chuanxiong Activating Blood and Unblocking Collaterals Capsule for Migraine with Blood Stasis and Collateral Obstruction Syndrome: Chuanxiong Rhizome 15 g, Safflower 9 g, Salvia Miltiorrhiza Root 15 g, Angelica dahurica Root 10 g, Tribulus Terrestris Fruit 9 g, Pueraria Root 15 g, Corydalis tuber 15 g.

Control Group 132: Modified Sanpian Decoction for Treating 80 Cases of Migraine: Large-sized Chuanxiong Rhizome 30 g, Angelica dahurica Root 1.5 g, Bupleurum Root 3 g, Honey-fried Cyperus Rhizome 6 g, Licorice Root 3 g, White Peony Root 15 g, Prunus Semen 3 g, Sinapis albae Semen 10 g.

Control Group 133: QufengHuoxue Decoction for Treating Vasomotor Headache: Chuanxiong Rhizome 30 g, White Peony Root 30 g, Chinese Angelica Root 15 g, Angelica Dahurica Root 15 g, Red Peony Root 15 g, Pubescent Angelica Root 12 g, Asarum Herb 6 g, Mentha Herb 8 g, Bombyx Batryticatus 10 g, Licorice Root 3 g.

Control Group 134: QufengDingtong Decoction for Treating Cluster Headache in 18 Cases: Chuanxiong Rhizome 30 g, White Peony Root 30 g, Gypsum Fibrosum 30 g, Salvia Miltiorrhiza Root 30 g, Angelica dahurica Root 15 g, Scutellaria baicalensis Root 15 g, Pheretima 15 g, Bombyx Batryticatus 15 g, Vitex Trifolia Fruit 15 g, Typhonium giganteum Rhizome 5 g, Safflower 6 g, Licorice Root 6 g.

Control Group 135: Modified Sanpian Decoction for Treating Headache in 175 Cases: Chuanxiong Rhizome 50 g, White Peony Root 25 g, Angelica dahurica Root 2.5 g, Sinapis albae Semen 15 g, Bupleurum Root 5 g, Prunus Semen 5 g, Cyperus Rhizome 10 g, Licorice Root 5 g.

Control Group 136: LvTongjie's Experience in Treating Headache with HuoxueShufeng Decoction: Chinese Angelica Root 12 g, Chuanxiong Rhizome 15 g, White Peony Root 12 g, Rehmannia Root 20 g, Cyperus Rhizome 9 g, Prunus Semen 9 g, Oyster Shell 20 g, Porphyra 20 g, Selfheal Spike 15 g, Pinellia tuber 9 g, Angelica dahurica Root 10 g, Cynanchum atratum Root 9 g, Sinapis albae Semen 15 g.

Control Group 137: XifengZhentong Decoction for the Treatment of Trigeminal Neuralgia: Gastrodia tuber 6 g, Uncaria Stem with Hooks 6 g (to be added later during decoction), Scorpion 3 g (ground into powder and taken orally), Centipede 1 piece (ground into powder and taken orally), Raw White Peony Root 12 g, Asarum Herb 3 g, Typhonium Rhizome 6 g, Bombyx Batryticatus 6 g, Tribulus Terrestris Fruit 6 g, Chrysanthemum 10 g, Chuanxiong Rhizome 6 g, Bupleurum Root 6 g, Saposhnikovia Root 6 g, Angelica dahurica Root 6 g, Notopterygium Root 6 g.

Control Group 138: Chief Physician Cai Youjing's Use of Xiongshao Analgesic Decoction in the Treatment of Vascular Headache. Xiongshao Analgesic Decoction: Chuanxiong Rhizome 30 g, White Peony Root 15 g, Angelica dahurica Root 10 g, Notopterygium Root 10 g, Bupleurum Root 10 g, Cyperus Rhizome 10 g, Uncaria Stem with Hooks 15 g, Nacre 30 g, Raw Licorice Root 3 g.

Control Group 139: Clinical Application of a Traditional Remedy for Injuries. The Remedy: Alcohol-fried Chinese Angelica Root 12 g, Purple Sage Root 15 g, Ground Beetle 10 g, Red and White Peony Roots 10 g each, Prepared Cyperus Rhizome 10 g, Jing Sanleng 10 g, Alcohol-fried Chuanxiong Rhizome 10 g, Fried Corydalis tuber 10 g, Zedoary Turmeric Rhizome 10 g, Panax Notoginseng Powder 3 g (taken orally), Scorpion 2.5 g (ground into powder and taken orally).

Control Group 140: Treatment of Migraine with Sanpian Decoction in 31 Cases. Sanpian Decoction: Chuanxiong Rhizome 30 g, Angelica dahurica Root, White Peony Root, Sinapis albae Semen, Bupleurum Root, and Bombyx Batryticatus 10 g each, Cyperus Rhizome, Scorpion, and Licorice Root 6 g each, Centipede 2 pieces (ground into powder and taken orally), Asarum Herb and Prunus Semen 3 g each.

Control Group 141: Treatment of Vascular Headache with Modified Siwu Decoction. Modified Siwu Decoction: Chinese Angelica Root 12 g, Chuanxiong Rhizome 15 g, Prepared Rehmannia Root 20 g, White Peony Root 12 g, Corydalis tuber 15 g, Asarum Herb 9 g, Abalone Shell 15 g, Bombyx Batryticatus 9 g, Uncaria Stem with Hooks 9 g, Spatholobus Stem 15 g.

Control Group 142: Clinical Observation of the Efficacy of Toufeng Powder in Treating Headache. Toufeng Powder: Chuanxiong Rhizome 30-60 g, Asarum Herb 3-9 g, Angelica Dahurica Root 30 g, Uncaria Stem with Hooks 20 g (to be added later during decoction), Gastrodia tuber 15 g, Corydalis tuber 30 g, White Peony Root 20 g.

Control Group 143: Treatment of 116 Cases of Refractory Headache with ToufengZhitong Decoction. ToufengZhitong Decoction: Bupleurum Root, Chrysanthemum Flower, Pueraria Root, Curcuma Root Tuber, Cicada Slough, and Gastrodia tuber 10 g each, Chuanxiong Rhizome 18 g, Angelica dahurica Root 12 g, Asarum Herb and Gastrodia tuber 6-10 g each, White Peony Root, and Haematitum 20 g.

Group 144 Comparison: Xiong Shao Scorpion Zhi Decoction for treating 40 cases of headache. Xiong Shao Scorpion Zhi Decoction: Chuanxiong Rhizome 10 g, White Peony Root 10 g, Angelica dahurica Root 10 g, Pueraria Root 10 g, Scorpion 5 g, Salvia miltiorrhiza Root 15 g.

Control Group 145: Treatment of 28 cases of refractory headache with XiongzhiQutong Decoction. XiongzhiQutong Decoction: Chuanxiong Rhizome 10-20 g, Angelica dahurica Root 12 g, White Peony Root 15 g, Salvia Miltiorrhiza Root 30 g, Asarum Herb 5 g, Bombyx Batryticatus 12 g, Scorpion 5 g, Honey-fried Licorice Root 8 g.

Control Group 146: Clinical observation of a self-formulated decoction for treating migraine without aura. Self-formulated headache formula: Notopterygium Root 10 g, Asarum Herb 3 g, Saposhnikovia Root 6 g, Angelica dahurica Root 12 g, Chinese Angelica Root 12 g, Scorpion 6 g, Astragalus Root 20 g, Photinia Leaf 12 g, Bombyx Batryticatus 10 g, Chuanxiong Rhizome 30 g, Pueraria Root 30 g, Licorice Root 6 g.

Control Group 147: Randomized parallel controlled study of the headache mixture for treating tension-type headache. Gastrodia Tuber, Processed Scorpion 10 g each, Scolopendra 1 piece, Vitex trifolia var. simplicifolia Leaf-fruit 15 g, Angelica dahurica Root 10 g, Pueraria Root 30 g, Corydalis tuber 10 g, Chuanxiong Rhizome 15 g.

Control Group 148: 60 cases of migraine were treated with SanpianZhitong Decoction. The composition of SanpianZhitong Decoction is as follows: upleurum Root, White Peony Root, Saposhnikovia Root, Chrysanthemum Flower, Notopterygium Root, Pinellia tuber, and Gardenia Fruit 12 g each. Chuanxiong Rhizome, Vitex Fruit, and Corydalis tuber 15 g each. Earthworm and Bombyx Batryticatus 10 g each. Salvia Miltiorrhiza Root 20 g. Panax Notoginseng Powder 8 g (to be taken after being dissolved in the decoction). Licorice Root 4 g.

Control Group 149: Treatment of 40 cases of muscle-contraction-type headache with Juantong Pills. Juantong Pills: Gastrodia Tuber 150 g, Chuanxiong Rhizome 100 g, Vitex trifolia var. simplicifolia Leaf-fruit 150 g, Corydalis tuber 100 g, Angelica dahurica Root 100 g, Prunella vulgaris Spike 120 g, Scorpion 150 g, Tribulus terrestris Fruit 100 g, Pueraria Root 120 g, Earthworm 120 g, Arctium lappa Fruit 100 g, Safflower 100 g, Platycodon grandiflorus Root 30 g.

Control Group 150: Treatment of 38 cases of migraine with Piantong Decoction. Piantong Decoction: Chuanxiong Rhizome 15 g, Notopterygium Root, Angelica dahurica Root, Corydalis tuber, Gastrodia Tuber, Bombyx Batryticatus 10 g each, Scorpion 5 g, Bupleurum Root, Cyathula Root 9 g each.

Control Group 151: Modified ChaihuShugan Powder for treating migraine. ChaihuShugan Powder: Bupleurum Root 9 g, Chuanxiong Rhizome 9 g, Cyperus Rotundus Rhizome 8 g, Aurantii Fructus Immaturus 9 g, Paeony Root 24 g, Licorice Root 15 g, Evodia rutaecarpa Fruit 9 g, Asarum Herb 3 g, Corydalis tuber 12 g.

Control Group 152: Treatment of 106 cases of migraine with Sanpiantong Capsules. Sanpiantong Capsules: Chuanxiong Rhizome 15 g, Angelica dahurica Root 9 g, Ligusticum sinense Oliv. Root 9 g, Asarum Herb 9 g, Vitex trifolia var. simplicifolia Leaf-fruit 12 g, Bupleurum Root 15 g, Earthworm 9 g, White Peony Root 12 g, Cyperus Rotundus Rhizome 9 g, Sinapis alba Seed 9 g.

Control Group 153: Randomized parallel controlled study of the liver-soothing, depression-relieving, and blood-activating method for treating migraine. Ingredients: Bupleurum Root 15 g, Chuanxiong Rhizome 15 g, Cyperus Rotundus Rhizome 9 g, Angelica sinensis Root 12 g, Bombyx Batryticatus 9 g, Uncaria rhynchophylla Hook 15 g, White Peony Root 12 g, Aurantii Fructus immaturus 15 g, Curcuma aromaticaSalisb. Root-tuber 9 g, Tangerine Peel 9 g, Achyranthes bidentata Blume Root 15 g, Licorice Root 8 g.

Control Group 154: Treatment of 50 cases of vascular headache with Chaixiong Decoction. Chaixiong Decoction: Bupleurum Root 15 g, Chuanxiong Rhizome 10 g, Gastrodia tuber 10 g, Angelica dahurica Root 10 g, Chinese Angelica Root 10 g, Salvia Miltiorrhiza Root 10 g, Earthworm 10 g, Cyperus Rhizome 10 g, Vitex Fruit 10 g, White Peony Root 10 g, Licorice Root 10 g, Indian Bread 10 g, Largehead Atractylodes Rhizome 10 g, Pinellia tuber 10 g.

Control Group 155: Treatment of 36 cases of vascular neurogenic headache with the method of nourishing blood, activating blood, expelling wind, and unblocking the collaterals. Ingredients: Chuanxiong Rhizome, Chinese Angelica Root, White Peony Root, Achyranthes Root, Pueraria Root 15 g each. Angelica dahurica Root, Ligusticum sinense Oliv. Root, Cicada Slough 10 g each. Asarum Herb 6 g. Scorpion 3 g (ground into powder and taken after mixing with the decoction).

Control Group 156: Modified Siwu Decoction for treating neuralgic headache. Siwu Decoction: Chinese Angelica Root 18 g, White Peony Root, Angelica dahurica Root, Saposhnikovia Root, Chrysanthemum Flower 12 g each, Prepared Rehmannia Root, Cyperus Rhizome 15 g each, Chuanxiong Rhizome 20 g, Asarum Herb 6 g, Ligusticum sinense Oliv. Root 10 g, Licorice Root 9 g.

Control Group 157: Treatment of 56 cases of migraine with Xue Tiao Shufeng Decoction. Xue Tiao Shufeng Decoction: White Peony Root 30 g, Chuanxiong Rhizome 30 g, Angelica dahurica Root 8 g, Bupleurum Root 6 g, Notopterygium Root 10 g, White Mustard Seed 10 g, Bushcherry Seed 6 g, Cyperus Rhizome 6 g, Licorice Root 3 g.

Control Group 158: Treatment of chronic headache with Toufukang Decoction. Toufukang Decoction: Chuanxiong Rhizome, Hawthorn Fruit, Prepared Rehmannia Root, White Peony Root 30 g each. Dogwood Fruit 25 g. Chinese Angelica Root, Cyperus Rhizome, White Mustard Seed, Angelica dahurica Root, Tangerine Peel 12 g each.

Control Group 159: Treatment of 56 cases of headache with Modified Xiaoyao Powder. Modified Xiaoyao Powder: Bupleurum Root 15 g, White Peony Root 12 g, Chinese Angelica Root 12 g, Largehead Atractylodes Rhizome 12 g, Peppermint 6 g, Perilla Leaf 15 g, Cyperus Rhizome 9 g, Angelica dahurica Root 10 g, Chuanxiong Rhizome 15 g, Motherwort Fruit 12 g, Licorice Root 8 g.

Control Group 160: Treatment of 24 cases of menstrual headache with Liver-Soothing and Blood-Activating Decoction. Liver-Soothing and Blood-Activating Decoction: Bupleurum Root 10 g, White Peony Root 12 g, Chuanxiong Rhizome 18 g, Chinese Angelica Root 10 g, Unprocessed Rehmannia Root 15 g, Peach Kernel 10 g, Gastrodia Tuber 10 g, Angelica dahurica Root 10 g, Salvia Miltiorrhiza Root 12 g, Cyperus Rhizome 10 g, Earthworm 10 g, Scorpion 10 g, Dragon Bone 22 g, Oyster Shell 22 g.

Control Group 161: Treatment of 56 cases of headache with Chinese medicine. Ingredients: Chuanxiong Rhizome 25 g, Notopterygium Root 15 g, Angelica dahurica Root 15 g, White Peony Root 15 g, Bombyx Batryticatus 15 g, Bupleurum Root 10 g, Vitex trifolia var. simplicifolia Leaf-fruit 15 g, Ligusticum sinense Oliv. Root 15 g, Licorice Root 10 g.

Control Group 162: Treatment of 39 cases of migraine with a self-formulated headache formula. Self-formulated headache formula: Chinese Angelica Root 12 g, Prepared Rehmannia Root 12 g, Chuanxiong Rhizome 12 g, White Peony Root 12 g, Asarum Herb 6 g, Corydalis tuber 15 g, Prunella vulgaris Spike 30 g, Uncaria rhynchophylla (Miq.) Jacks. 30 g, Cassia Seed 30 g, Suberect Spatholobus Stem 24 g, Angelica dahurica Root 12 g, Ligusticum sinense Oliv. Root 12 g, Bupleurum Root 12 g.

Control Group 163: Treatment of 36 cases of migraine with Headache Decoction. Headache Decoction: Chuanxiong Rhizome 12 g, Angelica dahurica Root, Notopterygium Root, Schizonepeta Tenuifolia Spike, Saposhnikovia Root 10 g each, Uncaria rhynchophylla Hook 20 g, White Peony Root 15 g, Scorpion, Bombyx Batryticatus, Peppermint 6 g each, Asarum Herb 4 g, Licorice Root 10 g.

Control Group 164: Observational study on the efficacy of a self-formulated Zhenshou Decoction (taken orally and used for external fumigation) in treating 116 cases of vascular headache. Self-formulated Zhenshou Decoction: Chuanxiong Rhizome 12 g, White Peony Root 20 g, Angelica dahurica Root 10 g, Arisaema Cum Bile 9 g, Panax Notoginseng 6 g, Bombyx Batryticatus 10 g, Chrysanthemum Flower 10 g, Tribulus terrestris Fruit 20 g.

Control Group 165: Integrated traditional Chinese and Western medicine treatment for 65 cases of vascular headache. Ingredients: Chuanxiong Rhizome 20 g, Angelica dahurica Root 15 g, Vitex trifolia var. simplicifolia Leaf-fruit 15 g, Tribulus terrestris Fruit 15 g, Bombyx Batryticatus 10 g, White Peony Root 10 g, Raw Oyster Shell 20 g, Asarum Herb 6 g.

Control Group 166: Treatment of 65 cases of migraine with Modified Wubai Decoction. Wubai Decoction: Angelica dahurica Root 6 g, White Mustard Seed 12 g, White Peony Root 12 g, Typhonium Rhizome 6 g, Bombyx Batryticatus 12 g, Chuanxiong Rhizome 15 g, Bushcherry Seed 9 g, Bupleurum Root 9 g, Earthworm 12 g, Ligusticum sinense Oliv. Root 12 g, Licorice Root 6 g, Asarum Herb 3 g.

Control Group 167: Treatment of 98 cases of migraine with Chinese medicine. Ingredients: Danshen Root 15 g, White Peony Root 18 g, Chuanxiong Rhizome and Tribulus terrestris Fruit 12 g each, Pueraria Root 30 g, Processed Typhonium Rhizome, Bombyx Batryticatus and Scorpion 6 g each, Earthworm 10 g, Arisaema Cum Bile 8 g, Honey-fried Licorice Root 5 g.

Control Group 168: Treatment of 76 cases of vascular headache with Dingtong Decoction. Dingtong Decoction: Magnetitum (Decocted First) 30 g, Raw White Peony Root, Danshen Root and Sunflower Receptacle 30 g each, Gastrodiaelata Blume 15 g, Corydalis Rhizome, Chuanxiong Rhizome, Red Peony Root, Earthworm and Bombyx Batryticatus 15 g each, Processed Arisaema Rhizome 10 g, Scorpion, Hirudo and Licorice Root 6 g each.

Control Group 169: Clinical observation of the treatment of vestibular migraine with the method of extinguishing wind and relieving spasms. Ingredients: Gastrodia elata Blume 30 g, Pueraria Root 45 g, White Peony Root 10 g, Rhodiola Root 20 g, Pinellia tuber 9 g, Fried Atractylodes macrocephalaKoidz. 20 g, Chuanxiong Rhizome 12 g, Corydalis Rhizome 10 g, Licorice Root 10 g.

Control Group 170: Treatment of 62 cases of vascular neurogenic headache with a self-formulated prescription. Self-formulated prescription: Chuanxiong Rhizome 20 g, Asarum Herb 3 g, Angelica dahurica Root 10 g, Chrysanthemum Flower 15 g, Bombyx Batryticatus 10 g, Gypsum Fibrosum 20 g, White Peony Root 20 g, Salvia Miltiorrhiza Root 15 g, Chinese Angelica Root 10 g, Saposhnikovia Root 10 g, Bupleurum Root 10 g.

Control Group 171: Treatment of 50 cases of migraine with XiongshenWubai Decoction. XiongshenWubai Decoction: Chuanxiong Rhizome 20 g, Salvia Miltiorrhiza Root 20 g, White Peony Root 15 g, Angelica dahurica Root 5 g, Bombyx Batryticatus 10 g, Chrysanthemum Flower 10 g, White Mustard Seed 6 g.

Control Group 172: Treatment of 92 cases of migraine with the method of activating blood, expelling wind, and resolving phlegm using Wuluo Tai. Wuluo Tai formula: Salvia Miltiorrhiza Root 15 g, White Peony Root 18 g, Chuanxiong Rhizome 12 g, Pueraria Root 30 g, Processed Typhonium Rhizome, Bombyx Batryticatus and Scorpion 6 g each, Earthworm 10 g, Tribulus terrestris Fruit 12 g, Arisaema Cum Bile 8 g, Honey-fried Licorice Root 5 g.

Control Group 173: Treatment of 32 cases of phlegm-stasis type migraine with TongmaiZhitong Formula. TongmaiZhitong Formula: Astragalus Root 20 g, Chinese Angelica Root 12 g, White Peony Root 30 g, Chuanxiong Rhizome 30 g, Hirudo 6 g, Angelica dahurica Root 9 g, Earthworm 6 g, Cinnamon Twig 6 g, Bupleurum Root 6 g, Pueraria Root 15 g, Gastrodia tuber 9 g, Cynanchum paniculatum Root 30 g, Wine-processed Rhubarb Root 30 g, Chrysanthemum Flower 9 g, Vitex trifolia var. simplicifolia Leaf-fruit 9 g.

Control Group 174: Clinical observation on the efficacy of GegenQuanchong Decoction in treating migraine. GegenQuanchongDecoction: Pueraria Root 30-60 g, White Peony Root 30-60 g, Licorice Root 10 g, Chuanxiong Rhizome 10-20 g, Angelica dahurica Root 15 g, Bupleurum Root 9 g, Haliotis Shell 30 g, Earthworm 20 g, Chrysanthemum Flower 10 g, Achyranthes Root 20 g, Uncaria Stem with Hooks 15 g (decocted later), Scorpion 2-6 g (ground into powder and taken with decoction).

Control Group 175: Treatment of 46 cases of blood stasis type migraine with Modified Xionglong Decoction. Modified Xionglong Decoction: Chuanxiong Rhizome 30 g, Pueraria Root 30 g, Corydalis Rhizome 30 g, Achyranthes Root 30 g, Earthworm 15 g, Asarum Herb 3 g, Angelica dahurica Root 9 g.

Decoction. TianmaGegenJiejin Decoction: Gastrodia Tuber 10 g, Pueraria Root 20 g, Chuanxiong Rhizome 10 g, White Peony Root 10 g, Earthworm 10 g, Corydalis Rhizome 10 g, Saposhnikovia Root 10 g.

Control Group 177: Treatment of 36 cases of refractory migraine with XiongshaoBan'ge Decoction. XiongshaoBan'ge Decoction: Chuanxiong Rhizome 30 g, White Peony Root 30 g, Pinellia tuber 30 g, Pueraria Root 30 g, Uncaria Stem with Hooks 30 g, Haliotis Shell 30 g, Asarum Herb 10 g, Angelica dahurica Root 10 g, Licorice Root 10 g. Decoct in water for oral administration, one dose daily. Scorpion 4 g, Scolopendra 2 pieces.

Control Group 178: Clinical observation of the combined treatment of Gegen Decoction and behavioral intervention for tension-type headache. Gegen Decoction: Pueraria Root 20 g, Chuanxiong Rhizome 15 g, Angelica dahurica Root 9 g, Curcuma Root-tuber 12 g, Mentha Haplocalyx Leaf 9 g (added later during decoction), Cinnamon Twig 6 g, White Peony Root 12 g, Scorpion 3 g.

Control Group 179: Treatment of vascular neurogenic headache with GegenQuanchong Decoction. GegenQuanchong Decoction: Pueraria Root 30-60 g, White Peony Root 30-60 g, Licorice Root 10 g, Chuanxiong Rhizome 10-20 g, Angelica dahurica Root 15 g, Bupleurum Root 9 g, Haliotis Shell 30 g, Earthworm 20 g, Chrysanthemum Flower 10 g, Achyranthes Root 20 g, Uncaria Stem with Hooks 15 g (decocted later), Scorpion 2-6 g (ground into powder and taken with decoction).

Control Group 180: Treatment of 50 cases of vascular neurogenic headache with Modified Zhitong Decoction. Modified Pain Relief Decoction: Chuanxiong Rhizome 15 g, Angelica dahurica Root 15 g, Ligusticum sinense Oliv. Root 15 g, Red Peony Root 15 g, White Peony Root 15 g, White Mustard Seed 15 g, Pueraria Root 15 g, Earthworm 10 g, Scorpion 10 g.

Control Group 181: Treatment with Headache Ning Formula. Headache Ning Formula: Chuanxiong Rhizome 40 g, Angelica dahurica Root 10 g, Asarum Herb 3 g, Mentha haplocalyx Leaf 30 g, Sinomenium acutum Root-stem 15 g, Gastrodia Tuber 20 g, Curcuma Root-tuber 12 g, Stir-fried Bombyx Batryticatus 12 g, Earthworm 12 g, Licorice Root 6 g.

Control Group 182: Flunarizine Hydrochloride Capsules (Xi'an Janssen Pharmaceutical Ltd., National Drug Standard H10930003, Product Batch Number: PBJ1100) 1.25 mg/kg-d.

After grouping, animals in each group were continuously gavaged for 7 days. The model group and the blank group were given an equal volume of distilled water. The body weight of the rats was measured, and the average body weight gain rate was calculated as: [(Average body weight after administration−Average body weight before administration)/Average body weight before administration]×100%.

### 2.3 Behavioral Testing

After the last nitroglycerin injection, behavioral observations were made and recorded.

### 2.3.1 Mechanical Pain Threshold Measurement

The von Frey filaments were used to measure the mechanical pain threshold of the rat's hind paw plantar surface. After the rats had acclimated to the environment (approximately 30 minutes), the von Frey filament was applied vertically to the central area of the hind paw skin, starting with the smallest unit and gradually increasing the force of the filament. A positive response was considered when the rat exhibited a paw withdrawal, escape response, or when it scratched the filament with its paw or licked the paw. The value corresponding to the positive response was recorded as the threshold for that measurement. Each rat was measured three times with at least a 5-minute interval between measurements, and the average of the three results was used as the threshold for subsequent statistical analysis.

### 2.3.2 Heat Pain Threshold Measurement

A heat radiation device was used to measure the heat pain threshold of the rat's hind paw, specifically recording the paw withdrawal latency. After the rat had adapted to the apparatus for about half an hour and stopped exhibiting exploratory behavior, the formal experiment began. The specific procedure was as follows: the radiation intensity was set to 50%, and the infrared radiation light spot was directed at the center of the rat's hind paw. A positive response was recorded when the rat exhibited a paw withdrawal or licking response. At this moment, the device's display automatically recorded the value as the rat's heat pain withdrawal latency. To prevent injury, the upper time limit for heat radiation was set to 20 seconds. Measurements were repeated three times with at least a 5-minute interval between each, and the average of the three results was used for subsequent statistical analysis.

### 2.4 Measurement of Serum Neurotransmitters and Related Indicators in Rats

On the 7th day post-administration, 24 hours after behavioral testing, all rats were intraperitoneally injected with Su Tai and atropine for anesthesia. Blood was collected from the abdominal aorta, left to stand for 30 minutes, and then centrifuged at 3500 rpm for 15 minutes to separate the serum, which was stored at −80° C. for later use. The levels of serotonin (5-HT) and calcitonin gene-related peptide (CGRP) in the rat serum were measured according to the instructions of the enzyme-linked immunosorbent assay (ELISA) kit.

### 2.5 Results

2.5.1 Effect of the Invention on the Body Weight of Chronic Migraine Rats: After administration, the majority of rats showed improvement in body weight loss caused by prolonged migraines. Notably, the group treated with the invention exhibited a significant improvement in body weight loss (P<0.05). See Table 5.

TABLE 5
Effect of the Invention on the Body Weight of Chronic Migraine Rats

| | Body Weight | Body Weight | Average BodyWeight Gain |
|---|---|---|---|
| Group | (0 d)/g | (9 d)/g | Rate (%) |
| Blank Group | 220.2 ± 2.73 | 279.5 ± 6.35 | 26.95 |
| Model Group | 220.7 ± 2.81 | 250.8 ± 13.26 | 13.63 |
| Invention Group1 | 221.5 ± 2.75 | 286.7 ± 12.86* | 29.44 |
| Invention Group2 | 220.8 ± 2.27 | 286.2 ± 6.05* | 29.62 |
| Invention Group3 | 220.3 ± 2.62 | 286.6 ± 7.99* | 30.10 |
| Control Group1 | 221.2 ± 1.75 | 286.5 ± 9.77* | 29.54 |
| Control Group2 | 219.3 ± 2.21 | 276.8 ± 9.54 | 25.72 |
| Control Group3 | 219.2 ± 2.27 | 284.2 ± 12.72 | 29.67 |
| Control Group4 | 220.8 ± 2.11 | 290.3 ± 13.14** | 31.46 |
| Control Group5 | 219.5 ± 1.50 | 281.3 ± 11.76 | 28.15 |
| Control Group6 | 219.0 ± 2.58 | 269.3 ± 17.21 | 22.97 |
| Control Group7 | 220.8 ± 2.73 | 291.3 ± 13.63** | 32.11 |
| Control Group8 | 220.8 ± 2.91 | 284.7 ± 17.58* | 29.21 |
| Control Group9 | 221.7 ± 2.49 | 288.3 ± 20.98* | 30.06 |
| Control Group10 | 219.7 ± 2.87 | 289.5 ± 17.67** | 31.79 |
| Control Group11 | 220.3 ± 2.62 | 289.8 ± 19.37** | 31.53 |
| Control Group12 | 220.2 ± 2.67 | 264.7 ± 13.72 | 20.23 |
| Control Group13 | 219.8 ± 2.54 | 279.2 ± 13.53 | 27.01 |
| Control Group14 | 221.2 ± 1.57 | 277.3 ± 10.25 | 25.38 |
| Control Group15 | 220.3 ± 2.49 | 265.7 ± 16.32 | 20.59 |
| Control Group16 | 220.0 ± 1.53 | 269.3 ± 8.78 | 22.41 |
| Control Group17 | 221.0 ± 1.29 | 268.7 ± 7.09 | 21.58 |
| Control Group18 | 221.2 ± 2.91 | 269.8 ± 10.57 | 21.99 |
| Control Group19 | 220.3 ± 1.60 | 283.8 ± 19.31 | 28.80 |
| Control Group20 | 219.7 ± 1.97 | 282.7 ± 15.53 | 28.69 |
| Control Group21 | 219.2 ± 2.79 | 285.3 ± 14.00* | 29.58 |
| Control Group22 | 220.8 ± 2.61 | 279.2 ± 18.85 | 26.43 |
| Control Group23 | 220.7 ± 2.75 | 281.2 ± 13.23 | 27.43 |
| Control Group24 | 220.3 ± 2.62 | 270.0 ± 16.20 | 22.54 |
| Control Group25 | 221.0 ± 2.08 | 286.2 ± 14.85* | 29.50 |
| Control Group26 | 219.5 ± 2.63 | 277.8 ± 19.50 | 25.61 |
| Control Group27 | 220.5 ± 2.87 | 270.0 ± 16.20 | 22.45 |
| Control Group28 | 219.5 ± 1.61 | 262.7 ± 18.22 | 19.23 |
| Control Group29 | 220.7 ± 2.29 | 258.3 ± 7.06 | 17.05 |
| Control Group30 | 220.7 ± 1.89 | 264.7 ± 17.08 | 19.95 |
| Control Group31 | 221.0 ± 3.00 | 278.7 ± 7.94 | 26.11 |
| Control Group32 | 22.07 ± 2.49 | 275.7 ± 11.45 | 24.94 |
| Control Group33 | 220.3 ± 2.62 | 263.8 ± 15.47 | 19.73 |
| Control Group34 | 221.7 ± 2.13 | 279.2 ± 16.51 | 25.95 |
| Control Group35 | 220.0 ± 2.89 | 270.2 ± 13.79 | 22.82 |
| Control Group36 | 221.0 ± 1.83 | 262.7 ± 16.21 | 18.87 |
| Control Group37 | 221.2 ± 2.27 | 262.0 ± 9.59 | 18.46 |
| Control Group38 | 219.5 ± 2.50 | 268.7 ± 18.41 | 22.41 |
| Control Group39 | 219.5 ± 2.57 | 270.2 ± 14.05 | 23.10 |
| Control Group40 | 220.3 ± 1.60 | 280.5 ± 19.66 | 27.31 |
| Control Group41 | 221.5 ± 2.36 | 267.2 ± 12.64 | 20.63 |
| Control Group42 | 220.7 ± 2.98 | 258.0 ± 13.31 | 16.92 |
| Control Group43 | 219.2 ± 2.54 | 258.2 ± 21.39 | 17.81 |
| Control Group44 | 220.0 ± 2.77 | 264.7 ± 19.57 | 20.32 |
| Control Group45 | 220.3 ± 2.81 | 260.7 ± 15.16 | 18.32 |
| Control Group46 | 220.7 ± 2.36 | 268.0 ± 16.24 | 21.36 |
| Control Group47 | 219.5 ± 2.87 | 264.7 ± 9.87 | 20.59 |
| Control Group48 | 221.5 ± 1.50 | 265.7 ± 16.60 | 19.95 |
| Control Group49 | 219.5 ± 2.81 | 270.7 ± 12.72 | 23.33 |
| Control Group50 | 220.0 ± 2.58 | 264.2 ± 14.58 | 20.09 |
| Control Group51 | 219.7 ± 2.62 | 262.5 ± 15.49 | 19.50 |
| Control Group52 | 220.3 ± 2.56 | 263.0 ± 19.59 | 19.36 |
| Control Group53 | 220.5 ± 1.50 | 257.3 ± 10.78 | 16.69 |
| Control Group54 | 220.0 ± 1.73 | 277.0 ± 21.84 | 25.91 |
| Control Group55 | 220.8 ± 2.34 | 261.3 ± 16.08 | 18.32 |
| Control Group56 | 221.2 ± 2.27 | 263.5 ± 8.02 | 19.14 |
| Control Group57 | 221.0 ± 2.00 | 276.3 ± 13.71 | 25.02 |
| Control Group58 | 220.5 ± 2.63 | 273.3 ± 14.32 | 23.95 |
| Control Group59 | 219.7 ± 2.87 | 262.2 ± 13.23 | 19.36 |
| Control Group60 | 220.7 ± 1.80 | 267.0 ± 12.49 | 21.00 |
| Control Group61 | 219.7 ± 1.70 | 255.5 ± 12.14 | 16.31 |
| Control Group62 | 220.7 ± 2.29 | 267.0 ± 18.95 | 21.00 |
| Control Group63 | 220.2 ± 2.48 | 281.2 ± 18.30 | 27.72 |
| Control Group64 | 219.7 ± 2.56 | 263.7 ± 21.69 | 20.05 |
| Control Group65 | 221.3 ± 2.49 | 270.5 ± 19.19 | 22.21 |
| Control Group66 | 221.2 ± 2.41 | 281.5 ± 13.34 | 27.28 |
| Control Group67 | 219.2 ± 2.41 | 275.3 ± 20.11 | 25.61 |
| Control Group68 | 219.7 ± 1.11 | 276.2 ± 15.29 | 25.74 |
| Control Group69 | 219.2 ± 2.61 | 271.3 ± 17.04 | 23.79 |
| Control Group70 | 219.7 ± 2.43 | 257.0 ± 21.64 | 17.00 |
| Control Group71 | 221.0 ± 2.31 | 276.7 ± 20.74 | 25.20 |
| Control Group72 | 220.7 ± 1.49 | 276.0 ± 16.38 | 25.08 |
| Control Group73 | 219.7 ± 2.75 | 275.2 ± 13.59 | 25.28 |
| Control Group74 | 219.5 ± 2.81 | 278.0 ± 17.83 | 26.65 |
| Control Group75 | 219.8 ± 2.41 | 287.2 ± 16.03* | 30.64 |
| Control Group76 | 219.3 ± 2.56 | 282.8 ± 11.48 | 28.45 |
| Control Group77 | 221.7 ± 2.05 | 262.2 ± 15.57 | 18.29 |
| Control Group78 | 220.0 ± 2.77 | 272.8 ± 17.97 | 24.00 |
| Control Group79 | 219.8 ± 2.73 | 282.0 ± 21.04 | 28.28 |
| Control Group80 | 219.5 ± 2.57 | 265.3 ± 18.14 | 20.87 |
| Control Group81 | 220.2 ± 2.85 | 281.7 ± 9.16 | 27.95 |
| Control Group82 | 219.0 ± 2.89 | 264.0 ± 22.57 | 20.55 |
| Control Group83 | 220.7 ± 2.43 | 284.7 ± 21.99* | 29.02 |
| Control Group84 | 219.5 ± 2.63 | 268.2 ± 20.51 | 22.19 |
| Control Group85 | 220.5 ± 2.36 | 285.2 ± 16.27* | 29.34 |
| Control Group86 | 220.5 ± 2.75 | 275.5 ± 22.90 | 24.94 |
| Control Group87 | 219.3 ± 2.49 | 281.0 ± 15.74 | 28.12 |
| Control Group88 | 219.2 ± 1.34 | 270.5 ± 19.65 | 23.42 |
| Control Group89 | 219.8 ± 2.27 | 277.7 ± 15.16 | 26.32 |
| Control Group90 | 220.8 ± 2.73 | 279.2 ± 10.42 | 26.43 |
| Control Group91 | 221.7 ± 2.21 | 255.3 ± 21.72 | 15.17 |
| Control Group92 | 220.0 ± 2.52 | 273.2 ± 16.24 | 24.18 |
| Control Group93 | 220.8 ± 1.57 | 282.0 ± 18.92 | 27.70 |
| Control Group94 | 219.2 ± 2.34 | 268.7 ± 17.27 | 22.60 |
| Control Group95 | 219.5 ± 2.63 | 271.7 ± 16.82 | 23.78 |
| Control Group96 | 220.2 ± 1.57 | 288.0 ± 22.57* | 31.11 |
| Control Group97 | 220.7 ± 1.80 | 275.7 ± 18.07 | 24.94 |
| Control Group98 | 220.5 ± 2.57 | 276.2 ± 18.86 | 25.26 |
| Control Group99 | 220.0 ± 1.41 | 257.5 ± 16.10 | 17.05 |
| Control Group100 | 220.0 ± 2.89 | 276.3 ± 18.53 | 25.12 |
| Control Group101 | 221.3 ± 1.97 | 271.5 ± 19.13 | 22.21 |
| Control Group102 | 220.3 ± 2.98 | 275.8 ± 19.82 | 24.80 |
| Control Group103 | 220.8 ± 2.27 | 271.2 ± 16.01 | 22.81 |
| Control Group104 | 220.0 ± 2.52 | 290.5 ± 19.62 | 32.05 |
| Control Group105 | 219.7 ± 2.98 | 263.5 ± 15.18 | 20.32 |
| Control Group106 | 221.2 ± 2.79 | 276.7 ± 15.68 | 25.11 |
| Control Group107 | 219.0 ± 2.89 | 275.5 ± 14.15 | 25.61 |
| Control Group108 | 220.2 ± 2.67 | 270.7 ± 18.08 | 23.23 |
| Control Group109 | 219.2 ± 1.67 | 255.0 ± 21.37 | 16.35 |
| Control Group110 | 220.2 ± 2.61 | 279.5 ± 20.74 | 26.95 |
| Control Group111 | 219.7 ± 2.49 | 272.7 ± 19.21 | 24.14 |
| Control Group112 | 219.2 ± 2.34 | 264.0 ± 16.75 | 20.46 |
| Control Group113 | 219.5 ± 1.38 | 279.2 ± 11.50 | 27.20 |
| Control Group114 | 219.2 ± 2.79 | 272.7 ± 15.06 | 24.43 |
| Control Group115 | 219.0 ± 2.77 | 277.3 ± 22.64 | 26.62 |
| Control Group116 | 219.7 ± 1.70 | 275.3 ± 14.57 | 25.33 |
| Control Group117 | 219.3 ± 1.97 | 274.0 ± 15.31 | 24.92 |
| Control Group118 | 221.1 ± 1.77 | 271.3 ± 12.42 | 22.67 |
| Control Group119 | 220.3 ± 2.75 | 268.3 ± 18.53 | 21.77 |
| Control Group120 | 220.5 ± 2.75 | 265.7 ± 23.59 | 20.50 |
| Control Group121 | 219.5 ± 2.06 | 273.8 ± 17.79 | 24.74 |
| Control Group122 | 220.3 ± 2.87 | 280.3 ± 15.72 | 27.22 |
| Control Group123 | 219.5 ± 2.22 | 268.7 ± 16.32 | 22.41 |
| Control Group124 | 220.3 ± 2.69 | 279.3 ± 23.07 | 26.76 |
| Control Group125 | 220.3 ± 2.43 | 277.0 ± 22.26 | 25.72 |
| Control Group126 | 220.8 ± 2.48 | 272.2 ± 23.69 | 23.26 |
| Control Group127 | 221.2 ± 2.97 | 265.0 ± 12.02 | 19.82 |
| Control Group128 | 219.3 ± 1.80 | 276.5 ± 19.44 | 26.06 |
| Control Group129 | 219.8 ± 2.29 | 284.2 ± 19.95 | 29.28 |
| Control Group130 | 221.5 ± 2.87 | 264.5 ± 23.55 | 19.41 |
| Control Group131 | 219.0 ± 2.94 | 279.5 ± 14.75 | 27.63 |
| Control Group132 | 220.8 ± 2.54 | 268.2 ± 11.99 | 21.45 |
| Control Group133 | 219.3 ± 2.69 | 265.3 ± 17.57 | 20.96 |
| Control Group134 | 220.3 ± 1.70 | 278.3 ± 12.79 | 26.12 |
| Control Group135 | 221.0 ± 2.83 | 268.0 ± 22.77 | 21.27 |
| Control Group136 | 221.0 ± 2.94 | 268.5 ± 19.32 | 21.49 |
| Control Group137 | 220.8 ± 2.29 | 269.5 ± 9.09 | 22.04 |
| Control Group138 | 220.8 ± 1.95 | 282.5 ± 7.23 | 28.02 |
| Control Group139 | 219.8 ± 2.19 | 279.5 ± 20.42 | 27.14 |
| Control Group140 | 221.0 ± 2.38 | 270.3 ± 20.28 | 22.31 |
| Control Group141 | 219.8 ± 2.67 | 274.7 ± 21.25 | 24.96 |
| Control Group142 | 220.0 ± 2.38 | 270.3 ± 17.81 | 23.14 |
| Control Group143 | 220.3 ± 2.21 | 284.8 ± 15.98* | 29.26 |
| Control Group 144 | 221.2 ± 1.86 | 268.0 ± 17.63 | 21.18 |
| Control Group145 | 220.2 ± 2.34 | 275.0 ± 21.23 | 24.91 |
| Control Group146 | 220.3 ± 2.69 | 280.3 ± 18.61 | 27.22 |
| Control Group147 | 221.5 ± 1.38 | 256.5 ± 17.38 | 15.80 |
| Control Group148 | 220.2 ± 2.54 | 271.3 ± 19.19 | 23.22 |
| Control Group149 | 220.2 ± 2.38 | 280.0 ± 14.74 | 27.27 |
| Control Group150 | 219.2 ± 2.91 | 269.3 ± 19.59 | 22.87 |
| Control Group151 | 221.2 ± 2.91 | 267.3 ± 19.50 | 20.86 |
| Control Group152 | 220.0 ± 1.29 | 267.0 ± 18.44 | 21.46 |
| Control Group153 | 221.3 ± 2.62 | 276.0 ± 14.79 | 24.70 |
| Control Group154 | 220.0 ± 2.71 | 273.3 ± 18.93 | 24.32 |
| Control Group155 | 219.7 ± 2.21 | 266.0 ± 13.39 | 21.09 |
| Control Group156 | 219.8 ± 2.11 | 276.7 ± 21.08 | 25.68 |
| Control Group157 | 219.8 ± 2.91 | 279.5 ± 20.43 | 27.14 |
| Control Group158 | 219.8 ± 2.85 | 268.0 ± 17.75 | 21.91 |
| Control Group159 | 221.2 ± 2.61 | 281.0 ± 18.92 | 27.05 |
| Control Group160 | 219.5 ± 2.93 | 287.3 ± 17.56* | 30.89 |
| Control Group161 | 220.5 ± 2.14 | 281.3 ± 17.78 | 27.57 |
| Control Group162 | 219.5 ± 1.71 | 271.8 ± 19.44 | 23.83 |
| Control Group163 | 220.2 ± 2.34 | 288.3 ± 11.36* | 30.95 |
| Control Group164 | 219.5 ± 1.98 | 278.8 ± 17.86 | 26.92 |
| Control Group165 | 219.8 ± 2.27 | 285.3 ± 22.14* | 30.17 |
| Control Group166 | 219.2 ± 1.86 | 280.3 ± 19.96 | 29.17 |
| Control Group167 | 220.7 ± 2.36 | 273.8 ± 19.54 | 24.17 |
| Control Group168 | 220.2 ± 2.79 | 267.2 ± 16.45 | 21.36 |
| Control Group169 | 219.7 ± 2.36 | 272.7 ± 14.43 | 24.24 |
| Control Group170 | 221.3 ± 2.29 | 271.2 ± 18.72 | 22.35 |
| Control Group171 | 219.7 ± 2.26 | 276.8 ± 17.93 | 27.36 |
| Control Group172 | 221.8 ± 2.91 | 271.3 ± 20.31 | 22.76 |
| Control Group173 | 219.8 ± 2.61 | 268.7 ± 17.51 | 22.60 |
| Control Group174 | 219.3 ± 1.11 | 275.2 ± 17.63 | 25.38 |
| Control Group175 | 220.0 ± 2.24 | 268.7 ± 15.15 | 22.04 |
| Control Group176 | 219.7 ± 1.89 | 266.5 ± 15.57 | 21.32 |
| Control Group177 | 221.8 ± 2.85 | 272.2 ± 10.78 | 22.70 |
| Control Group178 | 220.2 ± 1.67 | 276.5 ± 16.61 | 25.59 |
| Control Group179 | 218.8 ± 2.61 | 279.8 ± 17.41 | 28.25 |
| Control Group180 | 220.2 ± 2.19 | 286.8 ± 17.67* | 30.46 |
| Control Group181 | 220.5 ± 2.22 | 270.7 ± 19.73 | 22.77 |
| Control Group182 | 221.7 ± 2.79 | 277.7 ± 15.47 | 25.26 |

Note: Compared with the Model Group, *P < 0.05, **P < 0.01.

2.5.2 Effect of the Present Invention on Mechanical Pain Threshold in Chronic Migraine Rats: At 30 min and 1 h after the last nitroglycerin injection, the mechanical pain threshold was measured. Compared with the Blank Group, the Model Group showed a significantly decreased mechanical pain threshold (P<0.05). In contrast, the Invention Group exhibited a significantly increased mechanical pain threshold compared to the Model Group, with a statistically significant difference (P<0.05). These findings indicate that the Invention Group can effectively increase the mechanical pain threshold in chronic migraine rats. See Table 6.

TABLE 6
Effect of the Present Invention on Mechanical Pain Threshold in Chronic
Migraine Rats

| | Mechanical Pain | Mechanical Pain |
|---|---|---|
| | Threshold | Threshold |
| Group | at 30 min (g) | at 1 h (g) |
| Blank Group | 23.67 ± 0.26* | 23.35 ± 1.11* |
| Model Group | 14.16 ± 1.07 | 16.72 ± 1.47 |
| Invention Group1 | 22.92 ± 1.05 | 23.25 ± 1.53** |
| Invention Group2 | 21.12 ± 1.57** | 22.82 ± 1.31** |
| Invention Group3 | 23.30 ± 0.59** | 23.39 ± 1.23** |
| Control Group1 | 22.13 ± 1.92** | 22.49 ± 1.57** |
| Control Group2 | 20.80 ± 2.37** | 22.20 ± 1.52** |
| Control Group3 | 20.27 ± 2.41* | 19.42 ± 1.80 |
| Control Group4 | 19.09 ± 1.73 | 19.17 ± 1.79 |
| Control Group5 | 19.65 ± 2.91* | 20.29 ± 1.27* |
| Control Group6 | 19.26 ± 0.46 | 19.97 ± 1.47 |
| Control Group7 | 19.24 ± 1.26 | 19.86 ± 2.22 |
| Control Group8 | 19.56 ± 2.00 | 18.41 ± 1.40 |
| Control Group9 | 18.86 ± 1.15 | 19.48 ± 0.83 |
| Control Group10 | 20.27 ± 2.72* | 21.12 ± 1.39** |
| Control Group11 | 19.15 ± 1.96 | 19.40 ± 0.77 |
| Control Group12 | 19.51 ± 2.65 | 20.28 ± 1.90* |
| Control Group13 | 19.46 ± 1.75 | 19.32 ± 1.49 |
| Control Group14 | 19.09 ± 1.62 | 21.07 ± 1.01** |
| Control Group15 | 19.09 ± 1.72 | 19.32 ± 1.76 |
| Control Group16 | 19.31 ± 2.77 | 20.27 ± 1.34* |
| Control Group17 | 19.61 ± 2.13* | 20.24 ± 0.70* |
| Control Group18 | 20.24 ± 1.68* | 19.71 ± 1.65 |
| Control Group19 | 19.73 ± 2.47* | 20.24 ± 1.67* |
| Control Group20 | 20.56 ± 1.89** | 19.48 ± 2.15 |
| Control Group21 | 19.90 ± 2.61* | 19.96 ± 1.11 |
| Control Group22 | 19.51 ± 1.78 | 20.31 ± 0.80* |
| Control Group23 | 20.34 ± 2.03* | 20.52 ± 1.36** |
| Control Group24 | 19.84 ± 2.58* | 18.63 ± 0.99 |
| Control Group25 | 18.34 ± 2.79 | 18.62 ± 1.11 |
| Control Group26 | 18.95 ± 2.45 | 18.66 ± 1.80 |
| Control Group27 | 18.75 ± 1.66 | 18.41 ± 0.95 |
| Control Group28 | 18.03 ± 1.70 | 18.14 ± 0.77 |
| Control Group29 | 18.59 ± 2.08 | 17.97 ± 0.93 |
| Control Group30 | 18.61 ± 1.87 | 18.16 ± 1.79 |
| Control Group31 | 18.66 ± 1.85 | 18.67 ± 1.85 |
| Control Group32 | 18.33 ± 2.34 | 18.83 ± 1.30 |
| Control Group33 | 18.10 ± 1.04 | 18.80 ± 1.67 |
| Control Group34 | 19.27 ± 1.75 | 18.46 ± 1.15 |
| Control Group35 | 17.81 ± 1.19 | 18.42 ± 1.15 |
| Control Group36 | 17.90 ± 1.07 | 18.63 ± 1.20 |
| Control Group37 | 17.91 ± 2.19 | 18.33 ± 0.97 |
| Control Group38 | 18.66 ± 1.55 | 18.44 ± 1.95 |
| Control Group39 | 18.67 ± 1.75 | 18.27 ± 1.27 |
| Control Group40 | 17.95 ± 1.66 | 18.15 ± 1.07 |
| Control Group41 | 19.16 ± 1.52 | 18.40 ± 1.15 |
| Control Group42 | 18.68 ± 1.30 | 18.80 ± 1.01 |
| Control Group43 | 19.01 ± 1.68 | 18.53 ± 1.07 |
| Control Group44 | 18.51 ± 1.45 | 18.55 ± 0.91 |
| Control Group45 | 18.33 ± 1.31 | 17.99 ± 1.35 |
| Control Group46 | 17.99 ± 1.8 | 18.64 ± 1.26 |
| Control Group47 | 17.91 ± 1.17 | 18.08 ± 0.91 |
| Control Group48 | 17.74 ± 2.78 | 18.18 ± 0.79 |
| Control Group49 | 18.74 ± 1.6 | 18.69 ± 1.98 |
| Control Group50 | 17.86 ± 1.87 | 18.33 ± 1.04 |
| Control Group51 | 18.03 ± 1.38 | 18.96 ± 1.43 |
| Control Group52 | 18.80 ± 2.00 | 18.43 ± 1.18 |
| Control Group53 | 18.47 ± 1.02 | 18.53 ± 1.17 |
| Control Group54 | 18.75 ± 1.83 | 18.59 ± 1.39 |
| Control Group55 | 18.17 ± 2.26 | 18.47 ± 1.22 |
| Control Group56 | 17.74 ± 1.78 | 18.05 ± 1.21 |
| Control Group57 | 18.17 ± 1.16 | 17.89 ± 0.98 |
| Control Group58 | 17.67 ± 2.16 | 18.35 ± 1.09 |
| Control Group59 | 18.31 ± 1.08 | 17.74 ± 0.64 |
| Control Group60 | 18.50 ± 2.51 | 18.31 ± 1.02 |
| Control Group61 | 17.84 ± 2.37 | 18.97 ± 0.90 |
| Control Group62 | 18.61 ± 2.12 | 18.58 ± 1.36 |
| Control Group63 | 18.56 ± 0.96 | 18.25 ± 0.94 |
| Control Group64 | 18.10 ± 1.47 | 18.35 ± 0.98 |
| Control Group65 | 18.60 ± 1.82 | 18.12 ± 1.36 |
| Control Group66 | 18.48 ± 1.92 | 18.60 ± 1.51 |
| Control Group67 | 18.30 ± 0.96 | 18.73 ± 1.12 |
| Control Group68 | 17.86 ± 2.91 | 18.20 ± 0.89 |
| Control Group69 | 18.54 ± 2.33 | 18.45 ± 1.14 |
| Control Group70 | 18.58 ± 2.14 | 17.95 ± 1.06 |
| Control Group71 | 18.05 ± 2.19 | 18.67 ± 0.90 |
| Control Group72 | 18.76 ± 2.65 | 17.99 ± 0.57 |
| Control Group73 | 18.00 ± 2.58 | 18.43 ± 0.92 |
| Control Group74 | 18.91 ± 1.43 | 18.91 ± 0.63 |
| Control Group75 | 17.96 ± 1.83 | 18.30 ± 1.31 |
| Control Group76 | 18.43 ± 2.32 | 18.20 ± 1.07 |
| Control Group77 | 18.83 ± 1.42 | 17.92 ± 0.92 |
| Control Group78 | 17.72 ± 1.46 | 18.26 ± 1.13 |
| Control Group79 | 17.55 ± 2.96 | 18.21 ± 1.02 |
| Control Group80 | 18.41 ± 1.83 | 18.79 ± 1.64 |
| Control Group81 | 18.84 ± 2.98 | 18.54 ± 0.88 |
| Control Group82 | 17.27 ± 2.82 | 18.01 ± 0.60 |
| Control Group83 | 17.61 ± 0.77 | 18.46 ± 0.94 |
| Control Group84 | 17.47 ± 0.85 | 18.40 ± 1.63 |
| Control Group85 | 18.01 ± 2.61 | 18.11 ± 0.89 |
| Control Group86 | 17.28 ± 2.73 | 18.21 ± 0.67 |
| Control Group87 | 18.47 ± 2.18 | 18.46 ± 0.77 |
| Control Group88 | 18.89 ± 1.28 | 18.21 ± 0.95 |
| Control Group89 | 17.60 ± 1.24 | 18.08 ± 1.12 |
| Control Group90 | 18.94 ± 1.58 | 18.90 ± 1.49 |
| Control Group91 | 17.84 ± 1.57 | 18.90 ± 0.84 |
| Control Group92 | 18.23 ± 2.45 | 18.04 ± 0.85 |
| Control Group93 | 18.84 ± 1.96 | 18.48 ± 0.97 |
| Control Group94 | 18.38 ± 2.65 | 18.36 ± 1.04 |
| Control Group95 | 18.39 ± 1.93 | 18.36 ± 1.17 |
| Control Group96 | 18.32 ± 1.99 | 18.52 ± 0.61 |
| Control Group97 | 18.03 ± 2.62 | 18.90 ± 1.46 |
| Control Group98 | 20.39 ± 1.48** | 19.40 ± 1.61 |
| Control Group99 | 19.47 ± 2.47 | 20.12 ± 1.69* |
| Control Group100 | 19.68 ± 1.38* | 19.90 ± 0.88 |
| Control Group101 | 19.78 ± 2.68* | 20.36 ± 1.39* |
| Control Group102 | 19.17 ± 1.24 | 21.42 ± 0.53** |
| Control Group103 | 19.58 ± 0.77 | 19.62 ± 1.14 |
| Control Group104 | 19.61 ± 1.62* | 18.86 ± 0.79 |
| Control Group105 | 20.27 ± 0.61* | 20.88 ± 0.76** |
| Control Group106 | 19.32 ± 1.49 | 18.33 ± 1.13 |
| Control Group107 | 19.18 ± 1.14 | 18.84 ± 1.07 |
| Control Group108 | 17.34 ± 0.77 | 20.78 ± 0.53** |
| Control Group 109 | 18.06 ± 1.32 | 21.38 ± 0.92* |
| Control Group110 | 19.86 ± 1.75* | 19.42 ± 0.88 |
| Control Group111 | 17.44 ± 2.09 | 18.55 ± 0.71 |
| Control Group112 | 19.48 ± 1.78 | 18.93 ± 1.23 |
| Control Group113 | 19.17 ± 2.75 | 19.02 ± 1.24 |
| Control Group114 | 18.62 ± 2.07 | 18.43 ± 1.40 |
| Control Group115 | 19.13 ± 1.54 | 19.76 ± 1.26 |
| Control Group116 | 19.63 ± 0.62* | 18.84 ± 1.09 |
| Control Group117 | 19.20 ± 0.93 | 18.92 ± 1.54 |
| Control Group118 | 18.79 ± 1.11 | 18.28 ± 0.77 |
| Control Group119 | 18.16 ± 1.91 | 18.12 ± 0.70 |
| Control Group120 | 18.88 ± 0.71 | 18.14 ± 1.02 |
| Control Group121 | 18.11 ± 1.06 | 18.54 ± 0.86 |
| Control Group122 | 18.37 ± 1.86 | 18.06 ± 1.20 |
| Control Group123 | 18.34 ± 2.01 | 18.25 ± 1.16 |
| Control Group124 | 17.81 ± 2.57 | 18.60 ± 1.33 |
| Control Group125 | 18.34 ± 2.65 | 18.65 ± 0.51 |
| Control Group126 | 18.08 ± 2.51 | 18.31 ± 0.77 |
| Control Group127 | 18.27 ± 1.82 | 18.42 ± 0.97 |
| Control Group128 | 18.50 ± 1.58 | 18.40 ± 1.36 |
| Control Group129 | 19.41 ± 1.49 | 18.45 ± 1.03 |
| Control Group130 | 18.37 ± 1.94 | 18.07 ± 1.25 |
| Control Group131 | 18.65 ± 1.39 | 18.59 ± 1.62 |
| Control Group132 | 18.58 ± 1.64 | 18.37 ± 0.74 |
| Control Group133 | 18.95 ± 1.47 | 18.30 ± 1.22 |
| Control Group134 | 18.10 ± 2.57 | 18.71 ± 0.79 |
| Control Group135 | 17.95 ± 0.96 | 18.38 ± 1.03 |
| Control Group136 | 17.98 ± 1.85 | 18.16 ± 0.86 |
| Control Group137 | 18.22 ± 2.31 | 18.40 ± 1.57 |
| Control Group138 | 18.16 ± 1.94 | 17.93 ± 1.08 |
| Control Group139 | 18.91 ± 1.65 | 18.47 ± 1.15 |
| Control Group140 | 18.95 ± 0.49 | 18.41 ± 1.29 |
| Control Group141 | 18.59 ± 0.85 | 18.42 ± 0.81 |
| Control Group142 | 18.91 ± 1.47 | 18.30 ± 0.77 |
| Control Group143 | 17.70 ± 1.42 | 18.30 ± 0.84 |
| Control Group144 | 18.05 ± 0.57 | 18.11 ± 1.14 |
| Control Group145 | 17.23 ± 0.94 | 18.01 ± 1.36 |
| Control Group146 | 17.43 ± 1.52 | 18.39 ± 1.21 |
| Control Group147 | 18.66 ± 2.00 | 18.69 ± 1.06 |
| Control Group148 | 18.55 ± 2.50 | 18.33 ± 1.33 |
| Control Group149 | 18.51 ± 2.07 | 18.52 ± 0.93 |
| Control Group150 | 20.17 ± 1.64 | 21.26 ± 1.20** |
| Control Group151 | 19.17 ± 1.72 | 18.11 ± 1.09 |
| Control Group152 | 18.15 ± 2.41 | 18.98 ± 1.46 |
| Control Group153 | 18.29 ± 2.49 | 18.56 ± 1.06 |
| Control Group154 | 18.27 ± 2.10 | 18.26 ± 0.71 |
| Control Group155 | 18.11 ± 1.32 | 18.38 ± 0.91 |
| Control Group156 | 17.84 ± 2.29 | 18.54 ± 1.35 |
| Control Group157 | 17.89 ± 2.14 | 18.33 ± 0.69 |
| Control Group158 | 18.36 ± 2.28 | 18.30 ± 0.65 |
| Control Group159 | 18.14 ± 2.36 | 18.19 ± 0.99 |
| Control Group160 | 17.98 ± 2.32 | 18.63 ± 1.45 |
| Control Group161 | 18.85 ± 1.30 | 18.43 ± 1.03 |
| Control Group162 | 18.22 ± 2.28 | 18.03 ± 0.90 |
| Control Group163 | 17.85 ± 2.24 | 18.74 ± 0.79 |
| Control Group164 | 17.90 ± 1.97 | 18.23 ± 1.03 |
| Control Group165 | 17.59 ± 1.71 | 18.26 ± 0.88 |
| Control Group166 | 17.85 ± 2.24 | 18.38 ± 0.76 |
| Control Group167 | 18.20 ± 1.92 | 18.53 ± 1.09 |
| Control Group168 | 18.36 ± 1.13 | 18.42 ± 0.82 |
| Control Group169 | 18.37 ± 1.62 | 18.96 ± .05 |
| Control Group170 | 18.41 ± 2.26 | 18.47 ± 0.91 |
| Control Group171 | 17.75 ± 2.22 | 18.28 ± 0.83 |
| Control Group172 | 17.56 ± 0.91 | 18.05 ± 0.73 |
| Control Group173 | 17.69 ± 2.80 | 18.35 ± 1.18 |
| Control Group174 | 18.32 ± 2.28 | 18.25 ± 0.86 |
| Control Group175 | 17.37 ± 2.86 | 18.51 ± 0.75 |
| Control Group176 | 18.79 ± 2.87 | 18.84 ± 0.87 |
| Control Group177 | 18.61 ± 2.09 | 18.38 ± 0.94 |
| Control Group178 | 17.94 ± 2.21 | 18.38 ± 0.59 |
| Control Group179 | 18.76 ± 2.65 | 18.22 ± 0.42 |
| Control Group180 | 17.93 ± 1.66 | 18.53 ± 0.95 |
| Control Group181 | 20.84 ± 1.81** | 22.76 ± 0.92** |
| Control Group182 | 21.77 ± 2.40** | 22.48 ± 1.15** |

Note: Compared with the model group, *P < 0.05, P ** < 0.01

2.5.3 Effect of the Invention on Heat Pain Threshold in Chronic Migraine Rats: Heat pain threshold was tested 30 minutes and 1 hour after the final nitroglycerin injection. Compared to the blank group, the model group showed a significant shortening of the heat withdrawal latency (P<0.05). It was found that, compared to the model group, the Invention group effectively prolonged the heat withdrawal latency in chronic migraine rats, and the difference was statistically significant. See Table 7.

TABLE 7
Effect of the Invention on Heat Pain Threshold
in Chronic Migraine Rats

| | 30 min | 1 hour |
|---|---|---|
| | after: Grip | after: Grip |
| | withdrawal | withdrawal |
| Group | latency (s) | latency (s) |
| Blank Group | 16.18 ± 0.71 | 17.82 ± 0.89 |
| Model Group | 6.67 ± 1.02 | 10.06 ± 0.88 |
| Invention Group1 | 14.18 ± 1.14* | 17.03 ± 1.14** |
| Invention Group2 | 10.26 ± 1.94** | 13.73 ± 1.61** |
| Invention Group3 | 15.81 ± 1.09** | 16.68 ± 1.25** |
| Control Group1 | 12.06 ± 1.43** | 13.92 ± 0.47* |
| Control Group2 | 7.76 ± 0.84 | 9.64 ± 0.70 |
| Control Group3 | 11.16 ± 0.99** | 9.77 ± 0.37 |
| Control Group4 | 10.73 ± 0.08** | 9.31 ± 1.18 |
| Control Group5 | 10.61 ± 0.21** | 12.75 ± 0.33** |
| Control Group6 | 10.54 ± 0.40** | 12.72 ± 0.28** |
| Control Group7 | 7.47 ± 1.17 | 8.07 ± 0.85** |
| Control Group8 | 7.49 ± 0.50 | 12.89 ± 0.40** |
| Control Group9 | 10.75 ± 0.40** | 9.92 ± 0.30 |
| Control Group10 | 7.99 ± 1.35 | 9.84 ± 0.23 |
| Control Group11 | 8.28 ± 1.08 | 9.94 ± 0.22 |
| Control Group12 | 8.19 ± 0.65 | 10.03 ± 0.06 |
| Control Group13 | 8.23 ± 1.10 | 10.17 ± 0.11 |
| Control Group14 | 7.66 ± 0.53 | 9.84 ± 0.26 |
| Control Group15 | 8.36 ± 1.10 | 9.99 ± 0.10 |
| Control Group16 | 8.91 ± 0.46 | 10.00 ± 0.07 |
| Control Group17 | 8.07 ± 0.73 | 9.82 ± 0.08 |
| Control Group18 | 8.65 ± 0.55 | 9.82 ± 0.13 |
| Control Group19 | 7.51 ± 0.17 | 9.68 ± 0.09 |
| Control Group20 | 8.73 ± 0.97 | 12.73 ± 0.41** |
| Control Group21 | 8.54 ± 0.83 | 12.52 ± 0.52** |
| Control Group22 | 8.38 ± 1.06 | 9.89 ± 0.23 |
| Control Group23 | 8.37 ± 0.99 | 9.64 ± 0.11 |
| Control Group24 | 8.25 ± 0.57 | 10.01 ± 0.36 |
| Control Group25 | 7.99 ± 0.91 | 9.76 ± 0.08 |
| Control Group26 | 8.91 ± 0.54 | 10.01 ± 0.07 |
| Control Group27 | 8.41 ± 0.43 | 10.04 ± 0.06 |
| Control Group28 | 7.63 ± 1.40 | 9.97 ± 0.37 |
| Control Group29 | 8.11 ± 1.07 | 9.89 ± 0.23 |
| Control Group30 | 8.04 ± 0.78 | 9.94 ± 0.16 |
| Control Group31 | 7.94 ± 1.10 | 9.89 ± 0.23 |
| Control Group32 | 8.67 ± 0.74 | 10.12 ± 0.27 |
| Control Group33 | 8.47 ± 1.09 | 9.79 ± 0.29 |
| Control Group34 | 8.04 ± 1.26 | 9.79 ± 0.36 |
| Control Group35 | 8.82 ± 0.56 | 9.83 ± 0.31 |
| Control Group36 | 8.48 ± 0.83 | 9.89 ± 0.27 |
| Control Group37 | 7.76 ± 0.84 | 9.75 ± 0.36 |
| Control Group38 | 8.58 ± 1.30 | 9.69 ± 0.19 |
| Control Group39 | 7.83 ± 0.66 | 9.80 ± 0.19 |
| Control Group40 | 8.77 ± 0.60 | 9.90 ± 0.28 |
| Control Group41 | 8.23 ± 0.90 | 9.90 ± 0.26 |
| Control Group42 | 7.85 ± 0.83 | 9.86 ± 0.23 |
| Control Group43 | 8.16 ± 0.58 | 9.56 ± 0.05 |
| Control Group44 | 8.37 ± 0.36 | 9.85 ± 0.27 |
| Control Group45 | 8.38 ± 0.98 | 10.00 ± 0.23 |
| Control Group46 | 8.74 ± 1.08 | 10.13 ± 0.27 |
| Control Group47 | 8.53 ± 0.34 | 10.07 ± 0.19 |
| Control Group48 | 8.12 ± 0.76 | 9.93 ± 0.12 |
| Control Group49 | 8.08 ± 0.66 | 10.01 ± 0.06 |
| Control Group50 | 7.62 ± 0.42 | 9.99 ± 0.37 |
| Control Group51 | 8.47 ± 0.26 | 9.73 ± 0.19 |
| Control Group52 | 8.99 ± 0.69* | 9.80 ± 0.11 |
| Control Group53 | 8.14 ± 1.26 | 10.04 ± 0.41 |
| Control Group54 | 8.58 ± 0.71 | 10.00 ± 0.38 |
| Control Group55 | 8.60 ± 0.50 | 9.71 ± 0.06 |
| Control Group56 | 8.00 ± 0.71 | 10.23 ± 0.11 |
| Control Group57 | 8.60 ± 1.46 | 10.03 ± 0.24 |
| Control Group58 | 9.23 ± 0.61* | 9.95 ± 0.02 |
| Control Group59 | 8.07 ± 0.90 | 9.71 ± 0.10 |
| Control Group60 | 8.16 ± 1.04 | 9.99 ± 0.33 |
| Control Group61 | 8.52 ± 1.22 | 9.82 ± 0.20 |
| Control Group62 | 8.03 ± 0.99 | 9.87 ± 0.11 |
| Control Group63 | 7.62 ± 0.17 | 9.55 ± 0.01 |
| Control Group64 | 8.53 ± 0.81 | 10.08 ± 0.24 |
| Control Group65 | 7.82 ± 1.33 | 9.74 ± 0.21 |
| Control Group66 | 7.95 ± 0.24 | 9.98 ± 0.28 |
| Control Group67 | 9.14 ± 0.36* | 9.98 ± 0.10 |
| Control Group68 | 7.52 ± 0.54 | 9.75 ± 0.10 |
| Control Group69 | 9.15 ± 0.60* | 9.86 ± 0.25 |
| Control Group70 | 8.86 ± 0.69 | 9.88 ± 0.28 |
| Control Group71 | 8.58 ± 0.13 | 9.94 ± 0.18 |
| Control Group72 | 8.77 ± 0.52 | 10.09 ± 0.36 |
| Control Group73 | 8.40 ± 0.77 | 9.74 ± 0.24 |
| Control Group74 | 8.35 ± 1.20 | 9.92 ± 0.34 |
| Control Group75 | 8.35 ± 0.53 | 10.04 ± 0.33 |
| Control Group76 | 9.26 ± 0.21* | 9.78 ± 0.31 |
| Control Group77 | 7.62 ± 0.37 | 9.94 ± 0.30 |
| Control Group78 | 8.06 ± 1.40 | 9.70 ± 0.14 |
| Control Group79 | 8.78 ± 0.78 | 10.05 ± 0.06 |
| Control Group80 | 8.39 ± 1.17 | 10.09 ± 0.20 |
| Control Group81 | 7.13 ± 0.33 | 9.84 ± 0.24 |
| Control Group82 | 7.88 ± 0.94 | 9.89 ± 0.25 |
| Control Group83 | 8.42 ± 0.99 | 9.80 ± 0.17 |
| Control Group84 | 7.15 ± 0.50 | 9.78 ± 0.16 |
| Control Group85 | 8.74 ± 1.51 | 10.12 ± 0.17 |
| Control Group86 | 8.54 ± 1.07 | 10.07 ± 0.2 |
| Control Group87 | 8.15 ± 0.37 | 10.09 ± 0.27 |
| Control Group88 | 8.34 ± 1.06 | 10.05 ± 0.29 |
| Control Group89 | 7.57 ± 1.02 | 10.01 ± 0.17 |
| Control Group90 | 8.61 ± 0.90 | 10.14 ± 0.16 |
| Control Group91 | 8.88 ± 0.75 | 10.09 ± 0.23 |
| Control Group92 | 9.28 ± 0.60 | 9.80 ± 0.28 |
| Control Group93 | 8.55 ± 1.05 | 10.00 ± 0.30 |
| Control Group94 | 8.13 ± 1.00 | 9.88 ± 0.32 |
| Control Group95 | 7.79 ± 0.38 | 9.82 ± 0.17 |
| Control Group96 | 8.63 ± 0.70 | 9.69 ± 0.23 |
| Control Group97 | 7.56 ± 0.38 | 9.82 ± 0.24 |
| Control Group98 | 10.72 ± 0.14** | 13.84 ± 0.59** |
| Control Group99 | 7.96 ± 0.29 | 10.1 ± 0.13 |
| Control Group100 | 10.69 ± 0.22** | 9.83 ± 0.35 |
| Control Group101 | 10.77 ± 0.25** | 10.08 ± 0.34 |
| Control Group102 | 10.85 ± 0.19** | 9.84 ± 0.18 |
| Control Group103 | 10.49 ± 0.31** | 10.15 ± 0.21 |
| Control Group104 | 7.67 ± 1.05 | 12.82 ± 0.53** |
| Control Group105 | 8.05 ± 0.10 | 12.52 ± 0.29** |
| Control Group106 | 8.27 ± 0.40 | 9.13 ± 0.61 |
| Control Group107 | 8.60 ± 0.76 | 9.37 ± 0.80 |
| Control Group108 | 8.49 ± 0.69 | 12.83 ± 0.59** |
| Control Group109 | 8.57 ± 0.90 | 9.71 ± 0.42 |
| Control Group110 | 8.14 ± 0.97 | 9.43 ± 0.76 |
| Control Group111 | 7.77 ± 0.71 | 9.90 ± 0.41 |
| Control Group112 | 10.34 ± 0.22** | 9.81 ± 0.70 |
| Control Group113 | 10.55 ± 0.50** | 9.68 ± 0.42 |
| Control Group114 | 8.99 ± 0.53* | 9.72 ± 0.44 |
| Control Group115 | 8.42 ± 1.13 | 9.27 ± 0.26 |
| Control Group116 | 9.14 ± 0.14* | 9.36 ± 0.36 |
| Control Group117 | 8.36 ± 1.03 | 9.96 ± 0.22 |
| Control Group118 | 8.49 ± 0.49 | 9.73 ± 0.47 |
| Control Group119 | 8.29 ± 1.10 | 9.98 ± 0.12 |
| Control Group120 | 8.14 ± 1.08 | 9.27 ± 0.48 |
| Control Group121 | 7.75 ± 0.41 | 9.83 ± 0.10 |
| Control Group122 | 8.37 ± 0.55 | 9.49 ± 0.62 |
| Control Group123 | 8.28 ± 1.15 | 9.49 ± 0.44 |
| Control Group124 | 8.88 ± 0.90 | 10.00 ± 0.18 |
| Control Group125 | 7.71 ± 0.83 | 9.37 ± 0.32 |
| Control Group126 | 8.03 ± 0.63 | 9.69 ± 0.22 |
| Control Group127 | 9.35 ± 0.18** | 9.15 ± 0.21 |
| Control Group128 | 8.81 ± 0.33 | 9.47 ± 0.53 |
| Control Group129 | 7.54 ± 0.58 | 9.08 ± 0.23 |
| Control Group130 | 7.88 ± 0.32 | 12.38 ± 0.37** |
| Control Group131 | 8.98 ± 0.69 | 12.6 ± 0.42** |
| Control Group132 | 8.68 ± 0.60 | 9.6 ± 0.49 |
| Control Group133 | 8.79 ± 1.13 | 9.52 ± 0.47 |
| Control Group134 | 7.13 ± 0.22 | 9.46 ± 0.48 |
| Control Group135 | 7.68 ± 0.59 | 9.37 ± 0.19 |
| Control Group136 | 8.41 ± 1.11 | 9.75 ± 0.48 |
| Control Group137 | 8.12 ± 1.05 | 9.63 ± 0.60 |
| Control Group138 | 8.03 ± 0.78 | 9.63 ± 0.48 |
| Control Group139 | 8.05 ± 0.66 | 9.53 ± 0.33 |
| Control Group140 | 8.97 ± 0.47 | 9.86 ± 0.04 |
| Control Group141 | 9.03 ± 0.87* | 9.78 ± 0.26 |
| Control Group142 | 9.07 ± 0.62* | 9.84 ± 0.41 |
| Control Group143 | 8.2 ± 0.56 | 9.53 ± 0.43 |
| Control Group144 | 7.93 ± 0.69 | 10.18 ± 0.11 |
| Control Group145 | 8.87 ± 0.44 | 9.70 ± 0.47 |
| Control Group146 | 8.16 ± 0.67 | 9.29 ± 0.37 |
| Control Group147 | 8.10 ± 0.43 | 9.74 ± 0.51 |
| Control Group148 | 8.12 ± 1.21 | 9.79 ± 0.31 |
| Control Group149 | 7.66 ± 0.79 | 9.95 ± 0.23 |
| Control Group150 | 8.53 ± 0.34 | 9.96 ± 0.46 |
| Control Group151 | 7.76 ± 0.93 | 9.74 ± 0.44 |
| Control Group152 | 8.08 ± 1.3 | 9.33 ± 0.19 |
| Control Group153 | 7.80 ± 0.88 | 9.68 ± 0.01 |
| Control Group154 | 8.23 ± 0.62 | 9.45 ± 0.20 |
| Control Group155 | 7.30 ± 0.43 | 9.45 ± 0.42 |
| Control Group156 | 8.85 ± 0.41 | 10.04 ± 0.06 |
| Control Group157 | 7.88 ± 0.81 | 9.59 ± 0.61 |
| Control Group158 | 7.64 ± 0.50 | 9.56 ± 0.45 |
| Control Group159 | 7.31 ± 0.19 | 9.22 ± 0.25 |
| Control Group160 | 7.51 ± 0.74 | 9.67 ± 0.45 |
| Control Group161 | 8.45 ± 1.13 | 9.51 ± 0.17 |
| Control Group162 | 7.62 ± 0.61 | 10.00 ± 0.12 |
| Control Group163 | 8.59 ± 0.83 | 9.14 ± 0.1 |
| Control Group164 | 8.87 ± 0.64 | 9.97 ± 0.04 |
| Control Group165 | 8.13 ± 0.9 | 9.78 ± 0.27 |
| Control Group166 | 8.50 ± 0.75 | 9.30 ± 0.18 |
| Control Group167 | 8.00 ± 1.02 | 9.87 ± 0.08 |
| Control Group168 | 8.34 ± 1.18 | 9.76 ± 0.19 |
| Control Group169 | 7.75 ± 0.28 | 9.26 ± 0.31 |
| Control Group170 | 8.75 ± 0.93 | 9.15 ± 0.22 |
| Control Group171 | 8.13 ± 0.83 | 12.71 ± 0.27** |
| Control Group172 | 8.66 ± 1.08 | 9.19 ± 0.18 |
| Control Group173 | 7.79 ± 0.89 | 9.70 ± 0.23 |
| Control Group174 | 8.28 ± 0.40 | 9.30 ± 0.41 |
| Control Group175 | 7.75 ± 0.78 | 9.38 ± 0.38 |
| Control Group176 | 7.32 ± 0.58 | 9.81 ± 0.21 |
| Control Group177 | 7.83 ± 0.64 | 9.73 ± 0.45 |
| Control Group178 | 8.84 ± 0.19 | 9.40 ± 0.59 |
| Control Group179 | 8.70 ± 1.05 | 9.30 ± 0.25 |
| Control Group180 | 8.59 ± 0.82 | 9.19 ± 0.33 |
| Control Group181 | 10.57 ± 0.39** | 14.99 ± 0.68** |
| Control Group182 | 14.17 ± 0.44** | 15.47 ± 0.70** |

Note:
Compared with the model group, *P < 0.05, P** < 0.012.

5.4 Effect of the Invention on the 5-HT Levels in the Serum of Chronic Migraine Rats: The 5-HT content was measured 4 hours after the last nitroglycerin injection. Compared with the Blank Group, the 5-HT level in the serum of the Model Group significantly decreased (P<0.05). when compared with the Model Group, the 5-HT content in the serum of the Invention Group significantly increased, and the difference was statistically significant. It was found that the invention effectively increased the 5-HT content in the serum of chronic migraine rats. See Table 8.

TABLE 8
Effect of the Invention on Serum 5-HT
Levels in Chronic Migraine Rats

| Group | 5-HT (ng/ml) |
|---|---|
| Blank Group | 164.30 ± 3.55** |
| Model Group | 56.89 ± 6.17 |
| Invention Group1 | 145.80 ± 2.46** |
| Invention Group2 | 140.72 ± 3.70** |
| Invention Group3 | 158.75 ± 2.91 |
| Control Group1 | 133.11 ± 0.28** |
| Control Group2 | 100.65 ± 15.84* |
| Control Group3 | 104.94 ± 14.34* |
| Control Group4 | 90.71 ± 11.48 |
| Control Group5 | 96.09 ± 13.03 |
| Control Group6 | 97.26 ± 3.06 |
| Control Group7 | 98.85 ± 2.23 |
| Control Group8 | 100.32 ± 8.29 |
| Control Group9 | 104.46 ± 12.30* |
| Control Group10 | 94.07 ± 3.96 |
| Control Group11 | 102.06 ± 13.88* |
| Control Group12 | 99.14 ± 8.49 |
| Control Group13 | 91.79 ± 16.75 |
| Control Group14 | 91.41 ± 6.41 |
| Control Group15 | 94.02 ± 7.57 |
| Control Group16 | 90.5 ± 4.81 |
| Control Group17 | 99.41 ± 17.97 |
| Control Group18 | 102.39 ± 7.30* |
| Control Group19 | 98.75 ± 13.87 |
| Control Group20 | 100.99 ± 11.73* |
| Control Group21 | 106.47 ± 8.70** |
| Control Group22 | 90.62 ± 4.38* |
| Control Group23 | 92.86 ± 11.37 |
| Control Group24 | 92.79 ± 3.49 |
| Control Group25 | 108.34 ± 3.62** |
| Control Group26 | 87.92 ± 7.95 |
| Control Group27 | 101.79 ± 9.86* |
| Control Group28 | 101.75 ± 17.99* |
| Control Group29 | 94.22 ± 5.94 |
| Control Group30 | 90.21 ± 5.42 |
| Control Group31 | 93.34 ± 10.64 |
| Control Group32 | 107.11 ± 11.71** |
| Control Group33 | 95.03 ± 19.21 |
| Control Group34 | 101.87 ± 20.81* |
| Control Group35 | 98.8 ± 15.32 |
| Control Group36 | 103.19 ± 10.00* |
| Control Group37 | 93.29 ± 16.09 |
| Control Group38 | 108.73 ± 7.63** |
| Control Group39 | 98.99 ± 12.29 |
| Control Group40 | 94.17 ± 12.86 |
| Control Group41 | 92.56 ± 11.94 |
| Control Group42 | 101.24 ± 13.56* |
| Control Group43 | 92.98 ± 1.21 |
| Control Group44 | 99.26 ± 16.32 |
| Control Group45 | 95.69 ± 4.23 |
| Control Group46 | 106.1 ± 21.02** |
| Control Group47 | 105.88 ± 4.12** |
| Control Group48 | 101.98 ± 4.40* |
| Control Group49 | 92.38 ± 10.36 |
| Control Group50 | 93.02 ± 12.04 |
| Control Group51 | 92.91 ± 16.02 |
| Control Group52 | 90.34 ± 10.1 |
| Control Group53 | 94.67 ± 8.80 |
| Control Group54 | 85.4 ± 3.07 |
| Control Group55 | 102.76 ± 2.07* |
| Control Group56 | 95.19 ± 18.76 |
| Control Group57 | 100.21 ± 11.77 |
| Control Group58 | 106.76 ± 7.83** |
| Control Group59 | 96.46 ± 4.15 |
| Control Group60 | 102.23 ± 16.5* |
| Control Group61 | 97.44 ± 9.24 |
| Control Group62 | 93.46 ± 13.64 |
| Control Group63 | 91.42 ± 6.84 |
| Control Group64 | 103.42 ± 3.49* |
| Control Group65 | 82.69 ± 3.57 |
| Control Group66 | 109.42 ± 14.15** |
| Control Group67 | 87.26 ± 11.26 |
| Control Group68 | 100.83 ± 15.65* |
| Control Group69 | 96.62 ± 13.26 |
| Control Group70 | 96.19 ± 6.33 |
| Control Group71 | 106.11 ± 17.72** |
| Control Group72 | 106.4 ± 19.74** |
| Control Group73 | 99.98 ± 8.04 |
| Control Group74 | 100.6 ± 17.60 |
| Control Group75 | 104.74 ± 17.22* |
| Control Group76 | 96.98 ± 11.53 |
| Control Group77 | 102.67 ± 14.22* |
| Control Group78 | 93.23 ± 4.39 |
| Control Group79 | 101.23 ± 16.94* |
| Control Group80 | 114.95 ± 4.13** |
| Control Group81 | 98.26 ± 10.86 |
| Control Group82 | 104.24 ± 14.15* |
| Control Group83 | 92.13 ± 19.46 |
| Control Group84 | 99.58 ± 13.19 |
| Control Group85 | 110.35 ± 2.48** |
| Control Group86 | 94.88 ± 7.53 |
| Control Group87 | 97.69 ± 5.08 |
| Control Group88 | 105.59 ± 10.61** |
| Control Group89 | 84.34 ± 0.99 |
| Control Group90 | 101.02 ± 15.94* |
| Control Group91 | 97.43 ± 11.91 |
| Control Group92 | 93.82 ± 17.74 |
| Control Group93 | 105.64 ± 13.01** |
| Control Group94 | 108.16 ± 7.49** |
| Control Group95 | 97.38 ± 11.49 |
| Control Group96 | 99.16 ± 9.21 |
| Control Group97 | 98.37 ± 9.14 |
| Control Group98 | 110.08 ± 9.87** |
| Control Group99 | 93.44 ± 7.36 |
| Control Group100 | 88.05 ± 7.39 |
| Control Group101 | 93.80 ± 16.29 |
| Control Group102 | 104.31 ± 14.41* |
| Control Group103 | 95.80 ± 9.98 |
| Control Group104 | 106.86 ± 16.74** |
| Control Group105 | 94.95 ± 11.28 |
| Control Group106 | 97.39 ± 11.53 |
| Control Group107 | 103.14 ± 11.11* |
| Control Group108 | 93.20 ± 4.13 |
| Control Group109 | 104.86 ± 14.9* |
| Control Group110 | 103.76 ± 12.26* |
| Control Group111 | 101.44 ± 14.69* |
| Control Group112 | 108.45 ± 6.13** |
| Control Group113 | 99.01 ± 15.93 |
| Control Group114 | 96.61 ± 14.12 |
| Control Group115 | 100.67 ± 19.01* |
| Control Group116 | 108.65 ± 5.25** |
| Control Group117 | 107.24 ± 8.74** |
| Control Group118 | 98.5 ± 17.90 |
| Control Group119 | 97.67 ± 6.61 |
| Control Group120 | 104.73 ± 12.46* |
| Control Group121 | 92.55 ± 10.79 |
| Control Group122 | 95.91 ± 21.14 |
| Control Group123 | 96.98 ± 11.82 |
| Control Group124 | 110.61 ± 5.71** |
| Control Group125 | 87.84 ± 4.64 |
| Control Group126 | 108.32 ± 2.88** |
| Control Group127 | 93.98 ± 14.30 |
| Control Group128 | 90.62 ± 18.06 |
| Control Group129 | 94.94 ± 19.45 |
| Control Group130 | 109.96 ± 6.65** |
| Control Group131 | 104.99 ± 9.46* |
| Control Group132 | 107.19 ± 9.61** |
| Control Group133 | 99.28 ± 15.85 |
| Control Group134 | 97.34 ± 11.45 |
| Control Group135 | 89.34 ± 14.44 |
| Control Group136 | 97.92 ± 17.35 |
| Control Group137 | 99.76 ± 2.70 |
| Control Group138 | 112.14 ± 5.22** |
| Control Group139 | 95.35 ± 15.88 |
| Control Group140 | 102.83 ± 20.50* |
| Control Group141 | 96.48 ± 19.64 |
| Control Group142 | 103.04 ± 13.63* |
| Control Group143 | 103.45 ± 17.50* |
| Control Group144 | 94.91 ± 3.46 |
| Control Group145 | 106.79 ± 13.73** |
| Control Group146 | 109.19 ± 12.90** |
| Control Group147 | 95.89 ± 13.67 |
| Control Group148 | 100.33 ± 16.39 |
| Control Group149 | 108.37 ± 4.99** |
| Control Group150 | 92.09 ± 12.60 |
| Control Group151 | 100.60 ± 20.16 |
| Control Group152 | 101.93 ± 8.44* |
| Control Group153 | 88.36 ± 11.80 |
| Control Group154 | 107.97 ± 6.28** |
| Control Group155 | 100.66 ± 12.65* |
| Control Group156 | 104.87 ± 10.64* |
| Control Group157 | 88.37 ± 11.35 |
| Control Group158 | 99.97 ± 11.81 |
| Control Group159 | 97.94 ± 7.35 |
| Control Group160 | 106.8 ± 9.39** |
| Control Group161 | 100.34 ± 16.67 |
| Control Group162 | 97.74 ± 4.58 |
| Control Group163 | 92.97 ± 16.37 |
| Control Group164 | 93.72 ± 9.82 |
| Control Group165 | 115.31 ± 4.59** |
| Control Group166 | 104.15 ± 18.86* |
| Control Group167 | 109.16 ± 9.39** |
| Control Group168 | 89.00 ± 2.49 |
| Control Group169 | 87.32 ± 10.20 |
| Control Group170 | 96.79 ± 16.98 |
| Control Group171 | 112.66 ± 7.51** |
| Control Group172 | 97.33 ± 13.25 |
| Control Group173 | 100.07 ± 11.02 |
| Control Group174 | 90.83 ± 9.57 |
| Control Group175 | 104.85 ± 14.80* |
| Control Group176 | 95.78 ± 9.54 |
| Control Group177 | 101.87 ± 6.35* |
| Control Group178 | 97.22 ± 20.03 |
| Control Group179 | 101.01 ± 17.50* |
| Control Group180 | 98.2 ± 6.33 |
| Control Group181 | 130.01 ± 3.61** |
| Control Group182 | 155.2 ± 2.72** |

Note:
Compared with the Model Group, *P < 0.05, **P < 0.01.

2.5. Effect of the Invention on CGRP Levels in the Serum of Chronic Migraine Rats: The CGRP content was measured 4 hours after the last nitroglycerin injection. Compared with the Blank Group, the Model Group showed a significant increase in serum CGRP levels, with a statistically significant difference. It was found that the Invention Group significantly reduced the serum CGRP levels in chronic migraine rats compared to the Model Group (P<0.05). See Table 9.

TABLE 9
Effect of the Invention on CGRP Levels in
the Serum of Rats

| Group | CGRP ((pg/ml)) |
|---|---|
| Blank Group | 16.27 ± 1.24** |
| Model Group | 25.31 ± 1.07 |
| Invention Group1 | 19.04 ± 1.41** |
| Invention Group2 | 19.63 ± 1.31** |
| Invention Group3 | 17.57 ± 1.69** |
| Control Group1 | 19.82 ± 1.47** |
| Control Group2 | 19.96 ± 1.18** |
| Control Group3 | 20.09 ± 1.20** |
| Control Group4 | 20.48 ± 1.01* |
| Control Group5 | 21.02 ± 1.45 |
| Control Group6 | 21.08 ± 1.17 |
| Control Group7 | 21.29 ± 1.34 |
| Control Group8 | 20.95 ± 1.26* |
| Control Group9 | 21.40 ± 1.02 |
| Control Group10 | 21.47 ± 1.64 |
| Control Group11 | 21.52 ± 1.16 |
| Control Group12 | 21.67 ± 1.02 |
| Control Group13 | 21.52 ± 1.05 |
| Control Group14 | 21.05 ± 1.27 |
| Control Group15 | 21.24 ± 1.85 |
| Control Group16 | 20.93 ± 1.33* |
| Control Group17 | 20.94 ± 2.02* |
| Control Group18 | 21.07 ± 1.15 |
| Control Group19 | 20.74 ± 1.46* |
| Control Group20 | 21.68 ± 1.12 |
| Control Group21 | 21.33 ± 1.01 |
| Control Group22 | 21.63 ± 1.08 |
| Control Group23 | 21.54 ± 1.13 |
| Control Group24 | 20.28 ± 0.99** |
| Control Group25 | 20.06 ± 1.13** |
| Control Group26 | 21.55 ± 1.01 |
| Control Group27 | 21.76 ± 1.02 |
| Control Group28 | 20.01 ± 1.51** |
| Control Group29 | 20.52 ± 1.22* |
| Control Group30 | 21.51 ± 1.56 |
| Control Group31 | 20.14 ± 1.05** |
| Control Group32 | 20.48 ± 1.31* |
| Control Group33 | 20.54 ± 1.42* |
| Control Group34 | 20.60 ± 1.03* |
| Control Group35 | 21.29 ± 1.15 |
| Control Group36 | 20.38 ± 1.14** |
| Control Group37 | 21.92 ± 1.40 |
| Control Group38 | 20.24 ± 1.01** |
| Control Group39 | 20.43 ± 1.32* |
| Control Group40 | 21.12 ± 1.56 |
| Control Group41 | 21.52 ± 1.03 |
| Control Group42 | 21.47 ± 1.23 |
| Control Group43 | 20.86 ± 1.42* |
| Control Group44 | 20.81 ± 1.11* |
| Control Group45 | 20.32 ± 1.76** |
| Control Group46 | 21.10 ± 1.70 |
| Control Group47 | 20.84 ± 1.04* |
| Control Group48 | 21.71 ± 1.37 |
| Control Group49 | 20.40 ± 1.34* |
| Control Group50 | 20.28 ± 1.03** |
| Control Group51 | 21.08 ± 1.04 |
| Control Group52 | 20.02 ± 1.38** |
| Control Group53 | 20.19 ± 1.77** |
| Control Group54 | 21.43 ± 1.87 |
| Control Group55 | 20.69 ± 1.07* |
| Control Group56 | 20.87 ± 1.11* |
| Control Group57 | 20.16 ± 1.22** |
| Control Group58 | 20.51 ± 0.71* |
| Control Group59 | 20.46 ± 1.31* |
| Control Group60 | 20.15 ± 1.18** |
| Control Group61 | 21.31 ± 1.16 |
| Control Group62 | 21.19 ± 1.30 |
| Control Group63 | 21.63 ± 1.04 |
| Control Group64 | 20.47 ± 1.07* |
| Control Group65 | 20.69 ± 1.36* |
| Control Group66 | 20.54 ± 1.19* |
| Control Group67 | 21.08 ± 1.46 |
| Control Group68 | 21.87 ± 1.03 |
| Control Group69 | 20.72 ± 1.02* |
| Control Group70 | 21.05 ± 1.44 |
| Control Group71 | 21.46 ± 1.04 |
| Control Group72 | 20.61 ± 1.33* |
| Control Group73 | 21.57 ± 1.08 |
| Control Group74 | 20.57 ± 1.13* |
| Control Group75 | 21.15 ± 1.16 |
| Control Group76 | 20.59 ± 1.04* |
| Control Group77 | 21.27 ± 1.03 |
| Control Group78 | 21.52 ± 0.92 |
| Control Group79 | 20.57 ± 1.12* |
| Control Group80 | 20.48 ± 1.10* |
| Control Group81 | 20.48 ± 1.05* |
| Control Group82 | 20.87 ± 1.40* |
| Control Group83 | 21.14 ± 1.27 |
| Control Group84 | 20.54 ± 1.35* |
| Control Group85 | 20.37 ± 1.12** |
| Control Group86 | 20.46 ± 1.12* |
| Control Group87 | 20.45 ± 1.10* |
| Control Group88 | 21.22 ± 1.24 |
| Control Group89 | 20.96 ± 1.17* |
| Control Group90 | 21.65 ± 1.03 |
| Control Group91 | 21.18 ± 1.23 |
| Control Group92 | 21.27 ± 1.48 |
| Control Group93 | 21.43 ± 1.10 |
| Control Group94 | 21.09 ± 1.18 |
| Control Group95 | 20.59 ± 1.38* |
| Control Group96 | 21.18 ± 1.46 |
| Control Group97 | 20.29 ± 1.01** |
| Control Group98 | 21.60 ± 1.34 |
| Control Group99 | 21.72 ± 1.13 |
| Control Group100 | 20.78 ± 1.05* |
| Control Group101 | 20.50 ± 1.02* |
| Control Group102 | 21.41 ± 1.79 |
| Control Group103 | 20.61 ± 1.04* |
| Control Group104 | 20.70 ± 1.44* |
| Control Group105 | 20.30 ± 1.02** |
| Control Group106 | 20.44 ± 1.03* |
| Control Group107 | 21.40 ± 1.32 |
| Control Group108 | 20.53 ± 1.02* |
| Control Group109 | 20.38 ± 1.13** |
| Control Group110 | 21.31 ± 1.32 |
| Control Group111 | 20.53 ± 1.76* |
| Control Group112 | 20.56 ± 1.20* |
| Control Group113 | 20.13 ± 0.98** |
| Control Group114 | 21.19 ± 1.43 |
| Control Group115 | 20.59 ± 1.02* |
| Control Group116 | 20.93 ± 1.42* |
| Control Group117 | 21.34 ± 1.37 |
| Control Group118 | 20.61 ± 0.99* |
| Control Group119 | 20.87 ± 1.10* |
| Control Group120 | 20.35 ± 1.02** |
| Control Group121 | 20.62 ± 1.23* |
| Control Group122 | 21.18 ± 0.70 |
| Control Group123 | 21.45 ± 1.25 |
| Control Group124 | 21.41 ± 1.63 |
| Control Group125 | 21.63 ± 1.59 |
| Control Group126 | 20.27 ± 1.22** |
| Control Group127 | 21.19 ± 1.22 |
| Control Group128 | 21.09 ± 1.02 |
| Control Group129 | 20.65 ± 1.02* |
| Control Group130 | 21.13 ± 1.14 |
| Control Group131 | 20.54 ± 1.37* |
| Control Group132 | 21.62 ± 1.27 |
| Control Group133 | 21.66 ± 1.03 |
| Control Group134 | 20.21 ± 1.03** |
| Control Group135 | 20.86 ± 1.02* |
| Control Group136 | 20.68 ± 1.06* |
| Control Group137 | 20.56 ± 1.30* |
| Control Group138 | 21.39 ± 1.02 |
| Control Group139 | 20.02 ± 1.52** |
| Control Group140 | 20.31 ± 1.09** |
| Control Group141 | 20.66 ± 1.14* |
| Control Group142 | 21.40 ± 1.63 |
| Control Group143 | 21.03 ± 1.02 |
| Control Group144 | 20.77 ± 1.02* |
| Control Group145 | 21.25 ± 0.31 |
| Control Group146 | 20.52 ± 1.03* |
| Control Group147 | 21.16 ± 1.71 |
| Control Group148 | 21.41 ± 1.04 |
| Control Group149 | 21.74 ± 1.08 |
| Control Group150 | 21.14 ± 1.19 |
| Control Group151 | 21.36 ± 1.15 |
| Control Group152 | 20.59 ± 1.45* |
| Control Group153 | 21.15 ± 1.14 |
| Control Group154 | 21.33 ± 1.43 |
| Control Group155 | 21.42 ± 1.46 |
| Control Group156 | 20.72 ± 1.02* |
| Control Group157 | 21.77 ± 1.34 |
| Control Group158 | 20.17 ± 1.59** |
| Control Group159 | 21.93 ± 1.03 |
| Control Group160 | 21.14 ± 1.11 |
| Control Group161 | 21.12 ± 1.28 |
| Control Group162 | 21.01 ± 1.26* |
| Control Group163 | 20.62 ± 1.42* |
| Control Group164 | 20.79 ± 1.64* |
| Control Group165 | 20.27 ± 0.76** |
| Control Group166 | 21.20 ± 1.17 |
| Control Group167 | 21.60 ± 1.53 |
| Control Group168 | 21.54 ± 1.14 |
| Control Group169 | 20.51 ± 1.21* |
| Control Group170 | 20.98 ± 1.32 |
| Control Group171 | 21.55 ± 1.15* |
| Control Group172 | 21.48 ± 1.23 |
| Control Group173 | 21.65 ± 1.06 |
| Control Group174 | 20.80 ± 1.53* |
| Control Group175 | 20.89 ± 1.03* |
| Control Group176 | 21.37 ± 1.08 |
| Control Group177 | 21.48 ± 1.02 |
| Control Group178 | 20.86 ± 1.08* |
| Control Group179 | 21.34 ± 1.03 |
| Control Group180 | 21.03 ± 1.29 |
| Control Group181 | 20.54 ± 1.04* |
| Control Group182 | 18.19 ± 0.93** |

Note:
Compared with the Model Group, *P < 0.05, **P < 0.01

### Verification Example 3: Acute Toxicity Experiment

### 1. Materials

1.1. Experimental Animals: SPF grade ICR strain mice, 40 in total, half male and half female, with an average body weight ranging from 18.0 to 22.0 g. All mice were provided by the Animal Experiment Center of Zhejiang Chinese Medical University and were housed separately by gender. Prior to the formal commencement of the experiment, all mice were allowed a 7-day adaptation period. The conditions of animal husbandry, drinking water, and feed all meet national standards and relevant regulations.

1.2 Drugs: The headache relief and depression-regulating granules formulated in this invention, with a specification of 3.875 g of raw materials per gram.

2. Method: Considering that there are no toxic herbs in this prescription, the maximum tolerated dose (MTD) test is carried out according to the “Technical Guidelines for Acute Toxicity Studies of Medicinal Products” (2015 edition) and relevant experimental requirements.

2.1. Animal Grouping: After a 7-day adaptation feeding period, the mice are randomly divided into the TTL group (n=20) and the Control Group (n=20), with equal numbers of males and females.

2.2 Administration: The headache relief and depression-regulating granules are made from raw materials of the headache relief and depression-regulating prescription. These raw materials are extracted, concentrated, encapsulated, dried, and granulated. Through preliminary experiments, the maximum formulation concentration of the raw materials is approximately 30% (3 g of raw material/ml), with a maximum administration volume of 40 ml/kg. Mice are fasted for 6 hours prior to administration but are allowed to drink water. The administration is done twice a day, with a 4-hour interval, through oral gavage.

2.3 General Status Observation: After the last dose of the day, observe and record the general condition of the animals, including their fur color, limb movement, demeanor, drinking, defecation, and any other symptoms. Also, monitor for any toxic symptoms. If any are observed, record the symptoms in detail, including the time of onset, duration, and recovery. Continuous observation is conducted for 14 days.

2.4 Body Weight Observation: Record the body weight of the mice on Day 1, Day 2, Day 3, Day 5, Day 7, Day 10, and Day 14 after administration.

2.5 Food Intake Observation: On the day of administration (D0), a fixed amount of feed is provided to each group of mice. On D1, D2, D3, D5, D7, and D10, the remaining feed is weighed, and an equal amount of feed is added. On D14, the remaining feed is weighed again, and the average food intake per cage is calculated.

2.6 Anatomical Observation: The heart, lungs, trachea, kidneys, bladder, adrenal glands, brain, cerebellum, brainstem, spleen, thymus, testes, epididymis, ovaries, esophagus, stomach, small intestine, large intestine, liver, submandibular glands, pancreas, and thyroid of each group of mice are carefully dissected and separated, following strict anatomical procedures. If any changes in size, color, texture, or other characteristics are observed in the organs, they are fixed in 10% formaldehyde and stained with hematoxylin and eosin (HE) for histopathological examination. If no obvious abnormalities are observed macroscopically, organ fixation and histopathological examination are omitted.

2.7 Data Statistics and Analysis: SPSS 25.0 software is used for statistical processing and analysis of all data in this study. Continuous data are presented as mean±standard deviation. The chi-square test is used for intra-group analysis, and the corrected t-test is used for inter-group comparison. P<0.05 indicates that the difference is statistically significant.

### 3. Results

3.1 General Observation Results: In this MTD test, no obvious signs of stress response were observed in the TTL group and Control Group mice during oral administration and observation. No significant changes in appearance, fur color, or limb activity were noted. The excreta were normal, and no obvious toxic symptoms were observed. No mortality occurred in any of the groups.

### 3.2 Body Weight Observation Results

The body weight of each group of mice was recorded on Day 1, Day 2, Day 3, Day 5, Day 7, Day 10, and Day 14. Compared to the Control Group, the body weight of male and female mice in the TTL group slightly decreased throughout the experiment. The body weight changes in both groups were positively correlated with the length of the experiment, meaning that as the experimental days progressed, the body weight of both groups showed a trend of increase. However, the differences were not statistically significant (p>0.05). See Table 10.

TABLE 10
Acute Oral Toxicity Test of Headache
Relieving and Circulatory Activating
Granules on Mouse Body Weight (g, X̅ ± S)

| | | Sample | | |
|---|---|---|---|---|
| Gender | Days | Size | TTLGroup | Control Group |
| Male | 1 | 10 | 20.90 ± 1.15 | 21.21 ± 1.02 |
| | 2 | 10 | 23.23 ± 1.13 | 23.92 ± 1.17 |
| | 3 | 10 | 24.42 ± 1.15 | 25.50 ± 1.15 |
| | 5 | 10 | 27.53 ± 1.19 | 28.96 ± 1.18 |
| | 7 | 10 | 29.63 ± 1.16 | 31.01 ± 1.22 |
| | 10 | 10 | 33.01 ± 1.23 | 34.59 ± 1.19 |
| | 14 | 10 | 36.23 ± 1.20 | 37.63 ± 1.21 |
| Female | 1 | 10 | 20.18 ± 1.38 | 20.72 ± 1.16 |
| | 2 | 10 | 21.54 ± 1.35 | 22.45 ± 1.39 |
| | 3 | 10 | 22.42 ± 1.36 | 23.42 ± 1.45 |
| | 5 | 10 | 24.42 ± 1.33 | 25.11 ± 1.33 |
| | 7 | 10 | 25.30 ± 1.34 | 26.40 ± 1.31 |
| | 10 | 10 | 27.63 ± 1.31 | 28.43 ± 1.37 |
| | 14 | 10 | 29.24 ± 1.28 | 30.12 ± 1.41 |

Note:
Compared with the Control Group, *P < 0.05, **P < 0.01

### 3.3 Food Intake Variation Results

Compared with the Control Group, the food intake of Female mice in the TTL group showed slight fluctuations during the experiment. A significant difference in food intake was observed only between D1-D2 (the period immediately after dosing), with statistical significance (P<0.05). No significant differences were found during the rest of the period.

Compared with the Control Group, the food intake of Male mice in the TTL group showed slight fluctuations throughout the experiment, but no significant differences were observed (P>0.05). See Table 11.

TABLE 11
The Effect of Headache Relieving and Circulation
Promoting Granules on the Food Intake of Mice in
the Acute Oral Toxicity Test (g, X̅ ± S)

| | | TTL Group | Control Group |
|---|---|---|---|
| | | Average Daily | Average Daily |
| | | Food Intake per | Food Intake per |
| Gender | Days | Mouse (g) | Mouse (g) |
| Male | D0~D1 | 6.38 ± 0.60 | 6.31 ± 0.56 |
| | D1~D2 | 5.80 ± 0.45 | 6.21 ± 0.58 |
| | D2~D3 | 5.94 ± 0.58 | 6.18 ± 0.70 |
| | D3~D5 | 6.21 ± 0.58 | 6.16 ± 0.66 |
| | D5~D7 | 6.26 ± 0.47 | 6.33 ± 0.63 |
| | D7~D10 | 6.23 ± 0.40 | 6.17 ± 0.62 |
| | D10~D14 | 6.27 ± 0.54 | 6.20 ± 0.50 |
| Female | D0~D1 | 5.96 ± 0.60 | 5.77 ± 0.59 |
| | D1~D2 | 5.25 ± 0.69 | 5.97 ± 0.60 |
| | D2~D3 | 5.63 ± 0.64 | 5.97 ± 0.52 |
| | D3~D5 | 5.98 ± 0.57 | 5.88 ± 0.67 |
| | D5~D7 | 5.84 ± 0.49 | 5.92 ± 0.62 |
| | D7~D10 | 6.09 ± 0.64 | 6.00 ± 0.70 |
| | D10~D14 | 6.06 ± 0.55 | 6.09 ± 0.60 |

Note:
Compared with Control Group, *P < 0.05, **P < 0.01.

### 3.4 Dissection Results

The organs of the mice in each group, including the heart, liver, spleen, lungs, and kidneys, had normal color, size, and texture. No obvious pathological changes or abnormalities were observed in any of the major organs under gross examination.

4. Conclusion: The “Headache Relief and Qi-Blood Circulation Granules” is composed of seven medicinal herbs, including Chuanxiong and others. The formula is simplified, scientifically matched, and highly effective. The clinical dosage for adults (based on a 60 kg body weight) is approximately 1.55 g/kg of raw herbs per day. Through preliminary trials, the maximum concentration of the raw herbs for the formulation is about 30% (3 g of raw herbs/ml), with a maximum dose volume of 40 ml/kg, administered twice daily by oral gavage. In the ICR mice, the accumulated raw herb content given per day is 240 g/kg, which is 155 times the clinical dosage (240 g/1.55 g≈154.84).

Based on this, at the 155 times clinical dose, no significant acute toxicity symptoms were observed, indicating that the granules do not exhibit obvious toxicity at high doses and are safe for clinical use.

What is claimed is:

1. A traditional Chinese medicine composition for treating chronic headaches, wherein the composition comprises following components in parts by weight:
1200-1300 parts of Chuanxiong Rhizome, 570-670 parts of White Peony Root, 450-550 parts of Kudzu Root, 60-500 parts of Corydalis tuber processed with vinegar, 330-420 parts of Silkworm processed with honey and wheat bran, 320-420 parts of Cyperus Rhizome processed with vinegar, and 200-300 parts of Angelica dahurica Root.

2. A preparation method of the traditional Chinese medicine composition for treating chronic headaches according to claim 1, comprising following steps:
step 1) taking prescribed amounts of Chuanxiong Rhizoma and Cyperus Rhizome processed with vinegar, soaking them in water at a ratio of 1:5 (w/v) as a solvent, and performing steam distillation to extract a volatile oil, the volatile oil is encapsulated with a saturated β-cyclodextrin solution for later use,
step 2) centrifuging an extraction liquid obtained in the step 1) to remove impurities and set it aside,
step 3) taking a residue from the step 1) after volatile oil extraction, adding a prescribed amounts of Baishao Radix, Gegen Radix, and honey-fried Bombyx Batryticatus, and using water at a ratio of 1:10 (w/v) as a solvent for reflux extraction three times, each for 1 hour, combining an extracted liquids, centrifuging to remove impurities, and concentrating a solution to obtain a first extract,
step 4) Pulverize Corydalis tuber processed with vinegar and sieve it through a No. 3 sieve, taking a prescribed amounts of a vinegar-processed Yanhuosuo powder and Baizhi, using 60% ethanol at a ratio of 1:7 (w/v) as a solvent, and performing reflux extraction twice, each for 1.5 hours, combining an extracted liquids, filter out impurities using a plate-frame filter, and concentrating a solution to obtain a second extract,
step 5) combining the first and second extracts and performing spray drying to obtain a dry powder, adding the β-cyclodextrin-encapsulated volatile oil from the step 1) and mixing with excipients, processing a mixture into granules using a dry granulation method.

3. The preparation method of the traditional Chinese medicine composition for treating chronic headaches according to claim 2, wherein in the step 1), a soaking time is 0.5 hours, and a steam distillation time is 9.5 hours.

4. The preparation method of the traditional Chinese medicine composition for treating chronic headaches according to claim 2, wherein in the step 1), specific steps for encapsulating the volatile oil with the saturated β-cyclodextrin solution are as follows: the volatile oil and ethanol are mixed at a volume ratio of 1:4 to prepare a volatile oil ethanol solution, β-cyclodextrin is dissolved in water at a material-to-liquid ratio of 1 g:10 mL, stirred at 60° C. for 10 minutes, and then cooled to room temperature to prepare the saturated β-cyclodextrin solution, which is then transferred to a constant-temperature magnetic stirrer, the volatile oil ethanol solution is added dropwise under stirring at a β-cyclodextrin to volatile oil volume ratio of 8:1, with a magnetic stirring speed of 300 r/min and a temperature of 40° C., and an encapsulation is carried out for 180 minutes until a suspension state is formed, after cooling to room temperature, a mixture is refrigerated for 24 hours, then filtered and washed once with purified water, an obtained inclusion complex is naturally air-dried to obtain a dried encapsulated product.

5. The preparation method of the traditional Chinese medicine composition for treating chronic headaches according to claim 2, wherein in the step 3), a relative density of the first extract is 1.10±0.02 g/cm³, and a concentration temperature is 40-50° C.

6. The preparation method of the traditional Chinese medicine composition for treating chronic headaches according to claim 2, wherein in the step 4), are flux extraction temperature is 85° C., are lative density of the second extract is 1.00±0.02 g/cm³, and a concentration temperature is 40-50° C.

7. The preparation method of the traditional Chinese medicine composition for treating chronic headaches according to claim 2, wherein in the step 5), spray drying conditions are as follows: an inlet air temperature is 178° C., a peristaltic pump speed is 12 rpm, and are lative density of a medicinal liquid is 1.07 g/cm³.

8. The preparation method of the traditional Chinese medicine composition for treating chronic headaches according to claim 2, wherein in the step 5), the excipients used are dextrin and magnesium stearate, with an amount of dextrin being 5% of a total weight and an amount of magnesium stearate being 0.5% of the total weight.
